(19) United States
(12) Patent Application Publication
(10) Pub. No.: US 2008/0194419 A1
(43) Pub. Date: Aug. 14, 2008

(54) Genetic Association of Polymorphisms in the Atf6-Alpha Gene with Insulin Resistance Phenotypes

(75) Inventors: Steven J.R. Meex, Maasmechelen (BE); Carla J.H. Van Der Kallen, Sint-Oedenrode (NL); Tjerk W.A. De Bruin, Durham, NC (US); Marleen M.J. Van Greevenbroek, Tegelen (NL); Robert Vlietinck, Leuven (BE); Torik A.Y. Ayoubi, Genk (BE)

Correspondence Address: KNOBBE MARTENS OLSON & BEAR LLP, 2040 MAIN STREET, FOURTEENTH FLOOR, IRVINE, CA, 92614, US

(73) Assignee: Universiteti Maastricht, Maastricht (NL)

(21) Appl. No.: 11/915,113

(22) PCT Filed: May 5, 2006
(86) PCT No.: PCT/EP06/04216

§ 371 Date: Nov. 20, 2007

(30) Foreign Application Priority Data
May 23, 2005 (EP) 05447119.8

(51) Int. Cl. C40B 30/04; C12Q 1/68; C07H 21/00; G01N 33/566

(52) U.S. Cl. 506/9; 435/6; 536/24.33; 436/501

(57) ABSTRACT

The present invention relates to polymorphisms in the ATF6-alpha gene or locus which show genetic association with insulin resistance phenotypes, such as metabolic syndrome and type 2 diabetes. The present invention further relates to methods of genetic diagnosis of insulin resistance phenotypes comprising testing polymorphisms in the ATF6-alpha gene or locus and to reagents suitable for use in such methods. Further the invention relates to screening methods for compounds modulating the expression and/or function of ATF6-alpha gene and/or protein, which are useful in treatment and/or prevention of insulin resistance phenotypes.

### FIELD OF THE INVENTION

The present invention relates generally to the field of human genetics. In particular, the invention relates to polymorphisms in genes or loci, which show genetic association with phenotypes, including disease phenotypes. More specifically, the present invention relates to polymorphisms in the ATF6-alpha gene or locus which show genetic association with insulin resistance phenotypes. Accordingly, the present invention also relates to methods of genetic diagnosis of insulin resistance phenotypes comprising testing polymorphisms in the ATF6-alpha gene or locus and to reagents suitable for use in such methods. Further the invention relates to screening methods for compounds modulating the expression and/or function of ATF6-alpha gene and/or protein, which are useful in treatment and/or prevention of insulin resistance phenotypes.

### BACKGROUND OF THE INVENTION

Diabetes mellitus, long considered a disease of minor significance to world health, is now taking its place as one of the main threats to human health in the 21st century. The global figure of people with diabetes is set to rise from the current estimate of 150 million to 220 million in 2010 and 300 million in 2025. In particular, type 2 diabetes, also known as non-insulin dependent diabetes mellitus or NIDDM, may account for up to 90% of all cases of diabetes. Type 2 diabetes is typically a late-onset, chronically progressive disease. Insulin resistance, i.e., diminished response of peripheral tissues, e.g., liver, muscle and adipose tissues, to the biological action of insulin secreted by the pancreatic beta-cells, is a major etiological factor involved in development of type 2 diabetes. During development of insulin resistance, the body compensates for the diminished response of peripheral tissues to insulin by increasing production of insulin, but eventually the insulin levels are insufficient to effect the transport of glucose from bloodstream to peripheral tissues and hyperglycaemia results. Importantly, insulin resistance is not only an etiological factor in type 2 diabetes, but also contributes to other diseases or disease conditions, such as, e.g., metabolic syndrome (a cluster of metabolic abnormalities most of which constitute risk factors for cardiovascular diseases), obesity, and Familial Combined Hyperlipidemia.

Accordingly, finding novel ways to diagnose the risk of subjects to develop insulin resistance and related diseases, including type 2 diabetes, may be of great importance, e.g., when deciding on whether dietary, lifestyle changes, or drugs should be prescribed to a subject. Similarly, finding novel cellular pathways and/or proteins which contribute to development of insulin resistance and related diseases opens up not only the possibility to better understand these pathways but also to screen for compounds which modulate such pathways and the expression and/or function of proteins participating therein. Such compounds may be useful in therapy or prevention of insulin resistance and associated diseases.

Type 2 diabetes is a multifactoral and genetically heterogeneous disease with a strong environmental component. In determining the risk of developing type 2 diabetes, environmental factors such as food intake and exercise play an important role. Importantly, however, the risk profile of an individual is also strongly dependent on his or her genetic makeup. Accordingly, identifying novel susceptibility genes for insulin resistance and related diseases, including type 2 diabetes, is important, because genetic variation in such susceptibility genes may modulate the risk of subjects to develop insulin resistance and related diseases. Hence, when a susceptibility gene is identified, subjects may be screened for particular sequence variations, such as polymorphisms, which are known to be associated with increased or decreased risk of developing insulin resistance and related diseases. This provides a useful tool of genetic diagnosis which may predict whether a particular subject is at an increased risk of developing these conditions.

In addition, newly identified susceptibility genes for insulin resistance and related diseases and their gene products, as well as the cellular pathways in which they participate, represent important targets for therapeutic strategies aimed at treatment and/or prevention of these phenotypes. Hence, compounds which modulate the expression and/or function of proteins encoded by such susceptibility genes, or other proteins in the same cellular pathways, can be useful for treatment and/or prevention of the respective conditions. Accordingly, there exists a great need to identify novel susceptibility genes for insulin resistance and related diseases, e.g., type 2 diabetes, metabolic syndrome, or Familial Combined Hyperlipidemia. The present invention addressed this need to identify such susceptibility genes.

The endoplasmic reticulum (ER) is the site of synthesis, folding and modification of secretory and cell-surface proteins, as well as the resident proteins of the secretory pathway. The accumulation of unfolded proteins in the lumen of the ER, also referred to as ER stress, induces a coordinated adaptive program called the unfolded protein response (UPR), which alleviates ER stress by upregulating protein folding and degradation pathways in the ER and inhibiting protein synthesis. To date, three ER-resident transmembrane proteins have been identified as proximal sensors of the presence of ER stress: the kinase and endoribonuclease IRE1 (alpha and beta), the PERK kinase and the basic leucine-zipper transcription factor ATF6 (alpha and beta) (see Rutkowski et al. Trends Cell Biol 14: 20-26, 2004). IRE1 and PERK both comprise cytoplasmic serine/threonine kinase domains, wherein ER stress induces lumenal-domain driven homodimerization, autophosphorylation and activation. By contrast, the accumulation of unfolded proteins in the ER lumen leads to ATF6 transit to the Golgi complex, where it is cleaved, yielding a free cytoplasmic domain that is an active transcription factor (Ye et al. Mol Cell 6: 1355-1364, 2000). The combined effects of the activation of these molecules are an upregulation of genes encoding proteins that are involved in the secretory pathway, such as ER-resident chaperones and proteins involved in ER-associated protein degradation, and a downregulation of protein synthesis, reducing the influx of nascent proteins into the ER.

The transcription of genes downstream of ATF6 is upregulated by the translocation of the cytoplasmic domain of ATF6 to the nucleus. IRE1-dependent transcription is upregulated when the endoribonuclease domain of the activated IRE1 molecule catalyzes the removal of a small (26-nucleotide) intron from the mRNA of the gene encoding X-box-binding protein (XBP1). This splicing event creates a translational frameshift in XBP1 mRNA to produce an active transcription factor. PERK activation leads to the phosphorylation of the alpha subunit of the translation initiation factor elF2, which inhibits the assembly of the 80S ribosome and results in a general inhibition of protein synthesis.

Although IRE1, PERK and ATF6 are all activated by their dissociation from the ER chaperone BiP, the signalling pathways activated by each of these sensors are different, they regulate distinct parts of the UPR, and require unique lag times before they become fully activated. For example, the pathway activated most rapidly after exposure to ER stress, is repression of translation mediated by PERK. The immediate effect of this inhibition is to prevent further influx of nascent proteins into an already saturated ER lumen. Cleavage of ATF6 also follows fairly rapidly after exposure to stress. However, expression of the genes controlled by ATF6 protein requires nuclear translocation of its cytoplasmic domain, the induction of transcription and protein synthesis. The genes that are specifically induced by ATF6 include most of the prominent chaperones in the ER lumen, including BiP (=Grp78), TRA (=Grp94), calreticulin, and protein disulfide isomerase (PDI). Upregulation of protein folding capacity of the ER can be seen as a second stage of the UPR, following the inhibition of protein synthesis. IRE1 also initiates a pathway of transcriptional induction through XBP-1, but the full activation of this response is delayed relative to the activation of ATF6 pathway. The synthesis of XBP1 mRNA is upregulated by ATF6 and substantial amounts of XBP1 mRNA can only be generated after the induction of the initial transcriptional program of the UPR, ensuring that the IRE1-dependent pathway of the UPR is activated after the PERK and ATF6 pathways. One of the genes activated by XBP1 is the ER mannosidase EDEM, which is involved in the degradation of nascent misfolded ER glycoproteins. The stimulation of protein degradation through XBP-1 might, therefore, represent a third step of the UPR, following translational attenuation and increased chaperone synthesis.

It is known that obesity constitutes a major risk factor for development of insulin resistance and related diseases, such as type 2 diabetes and metabolic syndrome. For example, the prediabetic onset of insulin resistance is usually preceded by weight gain, and more than 80% of type 2 diabetics are overweight. Further, two publications have found a molecular link between obesity, ER stress and insulin resistance. For example, Özcan et al. (Science 306: 457-461, 2004) demonstrated that obesity causes endoplasmic reticulum stress (ER stress) and that activated inositol requiring kinase 1-alpha (IRE1-alpha), i.e., one of the sensors of ER stress, recruits tumour necrosis factor receptor-associated factor 2 (TRAF2), which activates c-Jun N-terminal kinase (JNK), which in turn serine phosphorylates insulin receptor substrate 1 (IRS-1), thereby downregulating insulin signalling. Downregulation of insulin signalling would lead to insulin resistance, and consequently to type 2 diabetes and other diseases. However, Özcan et al. 2004 only demonstrated the role of the UPR pathway initiated by IRE1-alpha in developing insulin resistance. He did not suggest that other UPR pathways, in particular the ATF6-dependent UPR pathway, may be involved in insulin resistance (e.g., as these pathways do not activate JNK). Further, Özcan et al. 2004 also did not suggest that genetic variation in genes encoding the proteins involved in regulation of UPR, and in particular in the ATF6-alpha gene, may show genetic association with insulin resistance or related diseases, such as type 2 diabetes or metabolic syndrome, in humans.

In another publication, Nakatani et al. (J Biol Chem 280: 847-851, 2005) demonstrated that ER stress and insulin resistance are linked because modifying the expression of oxygen regulated protein 150 (ORP150), a chaperone involved in the unfolded protein response, could ameliorate insulin resistance. Again, Nakatani et al. 2005 did not suggest any role for ATF6-dependent UPR pathway in development of insulin resistance or diabetes, and did not suggest that genetic variation in genes encoding the proteins involved in regulation of UPR, and in particular in the ATF6-alpha gene, may show genetic association with insulin resistance or related diseases, such as type 2 diabetes or metabolic syndrome, in humans.

### SUMMARY OF THE INVENTION

The present invention discloses the surprising finding that the ATF6-alpha gene represents a susceptibility gene for insulin resistance phenotypes and that individual polymorphisms and/or groups of two or more polymorphisms in the ATF6-alpha gene or locus may show genetic association with insulin resistance phenotypes. Exemplary insulin resistance phenotypes may include, among others, insulin resistance, type 2 diabetes and metabolic syndrome.

Further, the present invention discloses that testing of subjects for individual polymorphisms and/or for groups of two or more polymorphisms in the ATF6-alpha gene or locus can be useful in genetic diagnosis of insulin resistance phenotypes.

In a related aspect, the present invention relates to a method of genetic diagnosis of insulin resistance phenotypes which comprises testing of subjects for at least one polymorphism in the ATF6-alpha gene or locus.

In further aspects the invention relates to reagents, such as oligonucleotide primers, oligonucleotide probes and oligonucleotide arrays which are useful in methods of testing subjects for polymorphisms in the ATF6-alpha gene or locus. In a related aspect the invention relates to diagnostic kits comprising such reagents.

In yet further aspects, the invention relates to screening methods suitable for identifying molecules or compounds useful for treatment and/or prevention of insulin resistance phenotypes, wherein such molecules or compounds may bind to ATF6-alpha protein and/or modulate the expression and/or function of ATF6-alpha gene or protein.

In related aspects, the invention relates to molecules or compounds identified by such screening methods, as well as to the use of molecules or compounds for the treatment and/or prevention of insulin resistance phenotypes.

### BRIEF DESCRIPTION OF FIGURES

FIG. 1 illustrates measurement of ATF6-alpha mRNA levels in pre-adipocytes from subjects with different genotypes of the RS13401 polymorphism. X-axis: genotype of SNP RS13401, wherein “1” represents AA genotype, “2” represents AG genotype, “3” represents GG genotype. Y-axis: mean ATF6-mRNA Ct (cycle threshold) level+2 times standard error, measured by real time RT-PCR and normalized for 18S ribosomal RNA. Please note that higher Ct-values reflect lower expression levels.

FIG. 2 illustrates the cDNA sequence of human ATF6-alpha gene (also referred to in the text as SEQ ID NO: 1) and corresponds to the nucleic acid sequence present in the NCBI database of reference sequences (www.ncbi.nlm.nih.gov/entrez/) under RefSeq ID: NM_{—}007348.

FIG. 3 illustrates the sequences of oligonucleotide primers used to amplify segments of the ATF6-alpha gene comprising the RS13401 SNP polymorphism.

FIG. 4 illustrates the amino acid sequence of human ATF6-alpha gene (also referred to in the text as SEQ ID NO: 8) and corresponds to the amino acid sequence present in the NCBI database of reference sequences (www.ncbi.nlm.nih.gov/entrez/) under Refseq ID: NM_{—}007348.

FIG. 5 Increased levels of ATF-6 alpha protein in subjects having two G alleles of the RS13401 polymorphism.

ATF-6 alpha protein levels+SEM of GG polymorphism of RS13401 in subjects were compared to wildtype subjects, i.e. expressed as percentage of the wildtype protein (RS13401, having AA), which was set at 100%.

FIG. 6 Genetic association of RS1058405 SNP with total cholesterol and apolipoprotein B levels.

A) The level of ATF6-alpha protein in cultured primary human preadipocytes (n=11) was plotted versus total plasma cholesterol levels of the corresponding patient, in vivo.

B) The level of ATF6-alpha protein in cultured primary human preadipocytes (n=11) was plotted versus apolipoprotein B levels of the corresponding patient, in vivo.

### DETAILED DESCRIPTION OF THE INVENTION

As used herein, the singular forms “a”, “an”, and “the” include both singular and plural referents unless the context clearly dictates otherwise. By way of example, “a subject” refers to one or more than one subjects.

The disclosures of all patents and publications (including published patent applications) referenced in this specification are specifically incorporated herein by reference in their entirety to the same extent as if each such individual patent and publication were specifically and individually indicated to be incorporated by reference.

The recitation of numerical ranges by endpoints includes all numbers and fractions subsumed within that range.

As detailed in example 2, it is an observation of the present invention that in a case control study population (N=797) consisting of type 2 diabetes patients (N=210), subjects having impaired fasting glucose or impaired glucose tolerance (N=208) and normoglycaemic control subjects (N=379), the G allele of the single nucleotide polymorphism RS13401 in the human activating transcription factor 6 alpha (ATF6-alpha) gene is more prevalent in the type 2 diabetes patients and subjects having impaired fasting glucose or impaired glucose tolerance, than in the normoglycaemic control subjects. As detailed in example 1, it is a further observation of the invention that, in a population (N=125), representative for the general Dutch population and not artificially enriched for IFG/diabetic subjects, said G allele of the SNP RS13401 in the ATF6-alpha gene is also more prevalent among IFG (N=9) and diabetic (N=7) subjects than in normoglycaemic subjects (N=105). Analysis of these observations demonstrates that there exists a statistically significant correlation between the presence of at least one G allele of the single nucleotide polymorphism RS13401 in the ATF6-alpha gene of human subjects and increased susceptibility of such subjects to develop insulin resistance, as evidenced by impaired fasting glucose, impaired glucose tolerance and elevated fasting insulin, and to develop type 2 diabetes. Therefore, the SNP RS13401 in the ATF6-alpha gene displays genetic association with insulin resistance and type 2 diabetes.

Accordingly, the present invention demonstrates that the ATF6-alpha gene represents a susceptibility gene for insulin resistance phenotypes and that individual polymorphisms and/or groups of two or more polymorphisms in the ATF6-alpha gene or locus may show genetic association with insulin resistance phenotypes. Hence, the presence of particular alleles and/or genotypes of said individual polymorphisms and/or the presence of particular haplotypes of said groups of two or more polymorphisms may correlate with increased or decreased susceptibility of subjects to insulin resistance phenotypes. This is further corroborated by example 5, which demonstrates that RS2070150 SNP polymorphism in the ATF6-alpha gene shows genetic association, at least in the female population, with Familial Combined Hyperlipidemia (FCHL), another disease characterized by insulin resistance; and example 6, which demonstrates that RS1058405 SNP polymorphism in the ATF6-alpha gene shows genetic association with total plasma cholesterol and apolipoprotein B levels, which are important parameters in at least some diseases of insulin resistance, e.g., metabolic syndrome.

Accordingly, the present invention discloses that testing of subjects for individual polymorphisms and/or for groups of two or more polymorphisms in the ATF6-alpha gene or locus can be useful in genetic diagnosis of insulin resistance phenotypes. Preferably, said individual polymorphisms and/or said groups of two or more polymorphisms in the ATF6-alpha gene or locus will show genetic association with one or more insulin resistance phenotypes in at least one population. Preferably, the presence of particular alleles and/or genotypes of said individual polymorphisms and/or the presence of particular haplotypes of said groups of two or more polymorphisms in the ATF6-alpha gene or locus will correlate with increased or decreased susceptibility of subjects from said at least one population to one or more insulin resistance phenotypes. By means of an example and not limitation, the presence of particular alleles and/or genotypes of said individual polymorphisms and/or the presence of particular haplotypes of said groups of two or more polymorphisms in the ATF6-alpha gene or locus may correlate in subjects from said at least one population with increased or decreased susceptibility to develop one or more insulin resistance phenotypes, and/or with increased or decreased rate of progression of one or more insulin resistance phenotypes, and/or with increased or decreased clinical severity of one or more insulin resistance phenotypes and/or with increased or decreased response of one or more insulin resistance phenotypes to a given method of treatment.

Accordingly, in an aspect the present invention relates to a method of genetic diagnosis of insulin resistance phenotypes which comprises testing of a subject for at least one polymorphism in the ATF6-alpha gene or locus.

The term “genetic diagnosis” refers to diagnosing or to prognosticate a phenotype in a subject based on information comprised in the genetic material of said subject. A “phenotype” as used herein generally refers to any observable or measurable trait of an organism (e.g., an animal or human), and may in particular encompass a disease, e.g., a symptomatic disease, a disease state, a syndrome, or an asymptomatic (but detectable) pathological condition. Genetic diagnosis comprises the step of examining the genetic information of a subject in order to formulate the diagnosis or prognosis. In the present context, the “germline” genetic material of a subject is typically examined, i.e., the genetic material as inherited from the parents and as present in essentially every somatic cell. Genetic diagnosis may encompass, e.g., (i) diagnosing (i.e., establishing a diagnosis) of an existing phenotype in a subject; (ii) establishing whether particular genetically-determined etiological factors contribute to an existing phenotype in a subject (this may be useful, e.g., when selecting a treatment method directed at such etiological factors); (iii) establishing a prognosis of further course of an existing phenotype in a subject; or (iv) predicting whether a subject not yet having a phenotype has an increased or reduced chance of developing said phenotype (i.e., establishing whether a subject is predisposed, i.e., has increased susceptibility, to develop said phenotype).

The term “insulin resistance phenotype” as used herein in general refers to insulin resistance and diseases of insulin resistance. Sometimes the term also encompasses particular aspects or expressions related to these phenotypes, such as, e.g., likelihood and/or rate of progression from insulin resistance to a disease of insulin resistance; rate of progression of insulin resistance; rate of progression of a disease of insulin resistance; severity and/or clinical features of a disease of insulin resistance; or response of insulin resistance or of a disease of insulin resistance to a method of treatment.

In general, the term “insulin resistance” describes a pathological condition of a subject in which the biological responses to the action of insulin in the tissues of said subject are diminished. In other words, insulin resistance is a condition where circulating insulin produces a subnormal biological response. By way of example and not limitation, insulin resistance may result from a decreased quantity of insulin receptors in target tissues, e.g., liver, (skeletal) muscle and/or adipose tissues, or from defects in the insulin signalling pathways in cells of such target tissues. Insulin resistance may lead to impaired glucose metabolism and compensatory oversecretion of insulin by the pancreas. Accordingly, the term “insulin resistance” as used herein is also intended to encompass the usual diagnosis of insulin resistance made by any of a number of methods, including but not limited to: measurement of fasting glucose level, oral or intravenous glucose tolerance test, or measurement of fasting insulin level. Impaired fasting glucose (IFG), impaired glucose tolerance (IGT) and/or fasting hyperinsulinemia may serve as diagnostic markers for insulin resistance. IFG or IGT are often referred to as pre-diabetes.

A typical fasting blood glucose test measures the concentration of glucose in blood, serum or plasma of a subject after a fasting period of usually at least 10-12 hours. The normal range of whole blood glucose concentrations in this test is between 60 mg/dL (3.0 mmol/L) and 110 mg/dL (5.6 mmol/L). Impaired fasting glucose (IGF) is diagnosed when the whole blood glucose concentration of a subject is above 110 mg/dL (5.6 mmol/L) but less than 126 mg/dL (6.4 mmol/L). Diabetes mellitus may be diagnosed when the whole blood glucose concentration of a subject is above 126 mg/dL (6.4 mmol/L).

A typical oral glucose tolerance test (OGTT) is carried out as follows: after an overnight fasting period (e.g., 10 to 12 hours), a subject drinks a solution containing a known amount of glucose; blood is drawn before the subject drinks the glucose solution, and blood is drawn again every 30 to 60 minutes after the glucose solution is consumed for up to 3 hours. The normal ranges of whole blood glucose concentrations in a 75-gram oral glucose tolerance test are: between 60 mg/dL (3.0 mmol/L) and 110 mg/dL (5.6 mmol/L) after fasting; less than 200 mg/dL (10.1 mmol/L) at 1 hour after consumption of the glucose solution; less than 140 mg/dL (7.1 mmol/L) at 2 hours after consumption of the glucose solution. Impaired glucose tolerance (IGT) is diagnosed when the whole blood glucose concentration of a subject at 2 hours after consumption of the glucose solution is higher than 140 mg/dL (7.1 mmol/L) but less than 200 mg/dL (10.1 mmol/L). Diabetes mellitus may be diagnosed when the whole blood glucose concentration of a subject at 2 hours after consumption of the glucose solution is higher than 200 mg/dL (10.1 mmol/L).

A typical fasting blood insulin test measures the concentration of insulin in the plasma of a subject after a fasting period of usually at least 10-12 hours. The normal range of plasma insulin concentrations in this test is between 5 and 20 μU/mL. Fasting hyperinsulinemia is diagnosed above 20 μU/ml.

A diagnosis of insulin resistance may also be made using the euglycemic glucose clamp test. This test involves the simultaneous administration of a constant insulin infusion and a variable rate glucose infusion. During the test, the plasma glucose concentration is kept constant at euglycemic levels by measuring the glucose level every 5-10 minutes and then adjusting the variable rate glucose infusion to keep the plasma glucose level unchanged. Under these circumstances, the rate of glucose entry into the bloodstream is equal to the overall rate of glucose disposal in the body. The difference between the rate of glucose disposal in the basal state (no insulin infusion) and the insulin infused state, represents insulin mediated glucose uptake. In normal individuals, insulin causes brisk and large increase in overall body glucose disposal, whereas in type 2 diabetes subjects, this effect of insulin is greatly blunted, and is only 20-30% of normal. In insulin resistant subjects with either IGT or normal glucose tolerance, the rate of insulin stimulated glucose disposal is about half way between normal and type 2 diabetes. It is also known that there is a positive correlation between the level of fasting insulin or level of fasting glucose and the magnitude of the insulin resistance as measured by euglycemic glucose clamp tests and, therefore, this provides the rationale for using fasting glucose or fasting insulin levels as a measure of insulin resistance.

The term “diseases of insulin resistance” as used herein encompasses any diseases or disease states in which insulin resistance is commonly present and/or in which insulin resistance is, or is suspected to be, a factor contributing to the aetiology of the disease. In particular, the term encompasses insulin resistance syndrome (IRS), type 2 diabetes, metabolic syndrome, Familial Combined Hyperlipidemia (FCHL), polycystic ovary syndrome (PCOS), Familial hypertriglyceridemia, hypercholesterolemia, dyslipidemia, hepatic steatosis, obesity, primary diabetes mellitus (DM), hypertension, heart failure, atherosclerosis, cardiovascular diseases, lipodystrophy (genetic or acquired, e.g., HAART=highly active antiretroviral therapy), fatty liver, inflammation and Cushing's syndrome, and is preferably insulin resistance syndrome (IRS) or type 2 diabetes.

As such, the present invention relates particularly to a method of genetic diagnosis of an insulin resistance phenotype in a subject, comprising testing of a sample from said subject for at least one polymorphism in the gene or locus for activating transcription factor 6 alpha (ATF6-alpha), wherein said insulin resistance phenotype is chosen from the group comprising insulin resistance syndrome (IRS), type 2 diabetes, metabolic syndrome, familial combined hyperlipidemia (FCHL), hypercholesterolemia, familial hypertriglyceridemia, dyslipidemia, hepatic steatosis, obesity, polycystic ovary syndrome (PCOS), primary diabetes mellitus (DM), hypertension, lipodystrophy, fatty liver, inflammation and Cushing's syndrome, and preferably wherein said insulin resistance phenotype is insulin resistance syndrome (IRS) or type 2 diabetes.

The terms “insulin resistance syndrome” (IRS), “metabolic syndrome” or “syndrome X” may be used interchangeably herein and refer to a cluster of abnormalities which tend to co-occur in a subject and which represent major risk factors for the development of coronary artery disease (CAD), such as premature atherosclerotic vascular disease. These abnormalities in particular involve insulin resistance (impaired blood glucose), truncal obesity, high serum low density lipoprotein (LDL) cholesterol levels, low serum high density lipoprotein (HDL) cholesterol levels, high serum triglyceride levels, and high blood pressure (hypertension). According to the National Cholesterol Education Program of NIH, diagnosis of metabolic syndrome is made in the presence of any three of the following abnormalities: truncal obesity, defined as waist circumference of more than 102 cm for men and more than 89 cm for women; high serum levels of triglycerides, i.e., 150 mg/dL or higher; low serum levels of HDL cholesterol, i.e., below 40 mg/dL for men and below 50 mg/dL for women; high blood pressure, i.e., 130/85 mm Hg or higher; impaired fasting glucose, i.e., 110 mg/dL or higher. It has been proposed that at least some of these abnormalities may result from an attempt to compensate for insulin resistance (obesity may rather be a risk factor for insulin resistance).

The term “polycystic ovary syndrome” (PCOS) as used herein is intended to designate that etiologically unassigned disorder of premenopausal women, affecting 5-10% of this population, characterized by hyperandrogenism, chronic anovulation, defects in insulin action, insulin secretion, ovarian steroidogenesis and fibrinolysis. Women with PCOS frequently are insulin resistant and at increased risk to develop glucose intolerance or type 2 diabetes in the third and fourth decades of life (Dunaif et al. Clin Endocrinol Metab 81: 3299, 1996). Hyperandrogenism also is a feature of a variety of diverse insulin-resistant states, from type A syndrome, through leprechaunism and lipoatrophic diabetes, to type B syndrome, when these conditions occur in premenopausal women. It has been suggested that hyperinsulinemia per se causes hyperandrogenism.

Primary diabetes mellitus (DM) is classified as type 1 diabetes (also called juvenile onset DM or insulin dependent diabetes mellitus, IDDM) and type 2 diabetes mellitus (also called non-insulin dependent diabetes mellitus, NIDDM). Type 1 diabetes is a hormone deficient state, in which the pancreatic beta cells appear to have been destroyed by the body's own immune defence mechanisms. The destruction of beta cells in type 1 diabetes leads to the inability to produce insulin, and thereby chronic insulin deficiency. Patients with type 1 diabetes have little or no endogenous insulin secretory capacity and develop extreme hyperglycemia. Type 1 diabetes was fatal until the introduction of insulin replacement therapy—first using insulins from animal sources, and more recently, using human insulin made by recombinant DNA technology.

Type 2 diabetes mellitus (referred to herein as “type 2 diabetes”) is typically a chronic, life-long (i.e., progressing over several decades) disease characterized by insulin resistance. In clinical terms, insulin resistance is present when normal or elevated blood glucose levels persist in the face of normal or elevated levels of insulin. Symptoms may include excessive thirst, frequent urination, hunger, and fatigue. Typically, type 2 diabetes may be diagnosed when the fasting blood glucose concentration of a subject is above 126 mg/dL (6.4 mmol/L) on two occasions. Type 2 diabetes may also be diagnosed using OGTT when the blood glucose concentration of a subject at 2 hours after consumption of the glucose solution is higher than 200 mg/dL (10.1 mmol/L). Hyperglycaemia associated with type 2 diabetes can sometimes be reversed or ameliorated by diet changes or weight loss which may at least partially restore the sensitivity of the peripheral tissues to insulin. Treatment of type 2 diabetes frequently does not require the use of insulin. Therapy in type 2 diabetes usually involves dietary therapy and lifestyle modifications, typically for 6-12 weeks in the first instance. Features of a diabetic diet include an adequate but not excessive total calorie intake, with regular meals, restriction of the content of saturated fat, a concomitant increase in the polyunsaturated fatty acid content, and an increased intake of dietary fiber. Lifestyle modifications include the maintenance of regular exercise, as an aid both to weight control and also to reduce the degree of insulin resistance. If after an adequate trial of diet and lifestyle modifications, fasting hyperglycemia persists, then a diagnosis of “primary diet failure” may be made, and either a trial of oral hypoglycaemic therapy or direct institution of insulin therapy may be required to produce blood glucose control and, thereby, to minimize the complications of the disease. Progression of type 2 diabetes is associated with increasing hyperglycemia coupled with a relative decrease in the rate of glucose-induced insulin secretion. Therefore, for example, in late-stage type 2 diabetes there may be an insulin deficiency.

Familial Combined Hyperlipidemia (i.e., raised cholesterol, raised triglyceride levels, or a combination of both) affects 1% to 2% of individuals in Western society and is present in up to 20% of patients with premature coronary heart disease (CHD). The term “familial combined hyperlipidemia” (FCHL) was coined by Goldstein et al. (J Clin Invest 52: 1544-1568, 1973) to describe a pattern of lipid abnormalities in 47 Seattle pedigrees, which was simultaneously observed in two other data sets. FCHL was originally described as a dominant disorder with incomplete penetrance until the third decade and to primarily affect blood triglyceride levels and secondarily cholesterol levels. This was proposed because plasma triglyceride levels were bimodally distributed in the first-degree relatives of affected probands older than age 20 years and fewer than one half of the offspring of affected family members were hyperlipidemic. However, subsequent segregation analyses (Austin et al. Atherosclerosis 92: 67-77, 1992; Cullen et al. Arterioscler Thromb 14: 1233-1249, 1994) and genome-wide linkage studies (Aouizerat et al. Am J Hum Genet. 65: 397-412, 1999; Pajukanta et al. Am J Hum Genet. 64: 1453-1463, 1999) suggested a more complex inheritance pattern. The lipid profile in FCHL is characterized by increased plasma triglyceride or cholesterol levels, decreased HDL cholesterol levels, the presence of small, dense LDL particles, and elevated apolipoprotein (apo) B levels. These lipid abnormalities may also be present in persons with the metabolic syndrome, which is a major cause of CHD morbidity and mortality worldwide. Insulin resistance may be one of the characterizing features of FCHL, for example see Aitman et al. (Arterioscler Thromb Vasc Biol 17: 748-54, 1997), van der Kallen et al. (Atherosclerosis 164: 337-346, 2002) or Pihlajamaki et al. (Diabetes 50: 2396-2401, 2001).

The term “subject” as used herein refers to an animal, preferably a mammal, most preferably a human. The term “normal subject” or “healthy subject” refers to a subject not having a particular phenotype, e.g., insulin resistance phenotype.

The expression “testing of a subject for at least one polymorphism” refers to the step of detecting the presence or absence of particular allele(s) of said at least one polymorphism in the genetic material of the subject and/or determining one or both alleles of said at least one polymorphism in the genetic material of the subject. When both alleles of said at least one polymorphism are determined in the genetic material of the subject, the genotype of the subject for said at least one polymorphism can be constructed.

The term “polymorphism” as used herein refers generally to the occurrence of two or more alternative nucleotide sequences at a given site within a genome between individuals in a population. Said two or more alternative nucleotide sequences are referred to herein as “alleles”, “allelic forms” or “allelic variants” (used interchangeably herein) of a polymorphism. Typically, the first identified allele of a polymorphism is arbitrarily designated as the “reference allele” and subsequently identified alleles of said polymorphism are referred to as “alternative alleles”. Sometimes the allele occurring most frequently in a given population may be referred to as the “wildtype allele”. A polymorphism may have two known allelic forms (a “diallelic” polymorphism) or more than two known allelic forms, e.g., three (“triallelic”), four (“tetraallelic”), five (“pentaallelic”), six (“hexaallelic”), or more than six allelic forms. The site within a chromosome at which a polymorphism occurs is referred to herein as a “polymorphic site” or “polymorphic locus”. A polymorphic site may consist of a single nucleotide pair or may involve two or more nucleotide pairs.

A polymorphism may involve variation at one or more nucleotide positions in the nucleotide sequence of the reference allele, i.e., “reference sequence”. Such variation may include nucleotide substitutions (e.g., transitions and transversions), insertions, deletions, inversions, duplications, repeat length changes and other sequence rearrangements. For example, one useful category of polymorphisms found in eukaryotic genomes are repetitive elements, including simple sequence repeat (SSR) polymorphisms, variable number of tandem repeats (VNTR) polymorphisms, and polymorphisms due to insertion of repeat elements such as Alu or LINE. Simple sequence repeats (SSR), also known as short tandem repeats (STR) or microsatellites, are chromosomal regions consisting essentially of tandem repetitions of short nucleotide motifs. Such motifs are usually 2 to 6 nucleotides long and are repeated between two and several hundred times.

When a polymorphic site is defined by a single nucleotide pair, the polymorphism may involve a substitution, insertion or deletion of a single nucleotide in the reference sequence. Such polymorphism involving a single nucleotide pair is referred to as a “single nucleotide polymorphisms” and abbreviated “SNP” (plural “SNPs”). For example, if at a particular chromosomal location one individual from a population has an adenine (A) and another individual from the population has a thymine (T) at the same chromosomal location in the corresponding DNA strand, then such chromosomal location represents a polymorphic site or locus and the polymorphism is a SNP. Accordingly, in an embodiment, the polymorphisms for use in the present method of genetic diagnosis of insulin resistance phenotypes may be single nucleotide polymorphisms (SNP) located in the ATF6-alpha gene or locus. SNPs represent the most abundant type of sequence variation present in the genomes. For example, about 10 million SNPs are estimated in the human genome, on average one every 1.2 kb. A large number of SNPs from essentially all regions of the human genome have been and continue to be identified and are deposited in public or commercial databases. Exemplary databases comprise
dbSNP database (http://www.ncbi.nlm.nih.gov/SNP/);
The SNP Consortium database (http://snp.cshl.org/);
Human SNP database (http://www.broad.mit.edu/snp/human/);
GeneSNPs database (http://www.genome.utah.edu/genesnps/);
Human Genome Variation database (http://hgvbase.cgb.ki.se/); and
Celera Human Reference SNP database (www.celera.com).

In somatic cells of diploid organisms, corresponding polymorphic sites are present on each chromosome of a homologous chromosome pair. Therefore, diploid organisms may be homozygous or heterozygous for allelic forms of a given polymorphism. A diploid organism is “homozygous” for a given polymorphism when the two alleles of said polymorphism present on homologous chromosomes of said organism are identical. A diploid organism is “heterozygous” for a given polymorphism when the two alleles of said polymorphism present on homologous chromosomes of said organism are different. Accordingly, the terms “homozygous” and “heterozygous” as used herein refer to the presence of identical or different, respectively, alleles of a given polymorphism in an individual or a subject.

The pair of alleles present at a given polymorphic site in the genetic material of an individual or a subject represents the “genotype” of said individual or subject at said polymorphic site or for said polymorphism. Different alleles of a polymorphism may be assigned distinct symbols, e.g., numerical or alphabetical characters, to facilitate the presentation and storage of information about genotypes of different individuals or subjects for one or more polymorphisms. Such information may be stored on a computer medium and integrated in a database.

Typically, the two allelic forms of a given polymorphism present in a diploid organism are genetically determined, i.e., transmitted from the parents to said organism. Occasionally, such alleles may originate from a spontaneous recombination or mutation event, or by artificial means, e.g., by a targeted genetic manipulation. For example, alleles of SSR polymorphisms may undergo a change in the number of repetitions of the basic nucleotide motif upon transmission from a parent to an offspring.

The term “polymorphism” further encompasses polymorphisms having a population frequency of the least abundant allele of 1% or more, as well as polymorphisms having a population frequency of the least abundant allele of less than 1%. “Allele frequency” refers to the frequency with which a particular allele is present at a given polymorphic locus in a given population. It refers explicitly to the frequency of an allele in a population and not to the population frequency of individual genotypes comprising said allele.

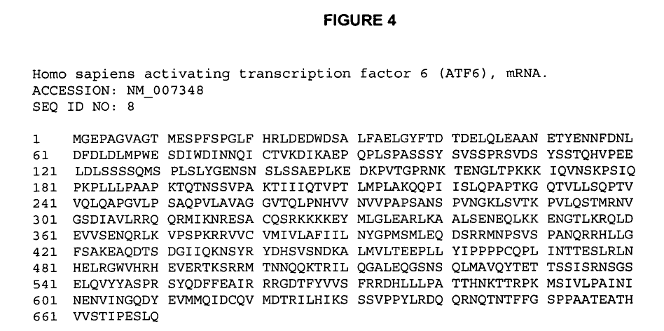

The term “polymorphism” further encompasses polymorphisms within genes, as well as polymorphisms in chromosomal regions which do not correspond to a gene or portion of a gene. Polymorphisms located in genes may be found in regulatory elements (e.g., promoters and enhancers) or in transcribed sequences including exons, introns, coding regions and sequences specifying the 5′ untranslated region (5′ UTR) and 3′ untranslated region (3′ UTR) regions. Accordingly, in an embodiment, the polymorphisms for use in the present method of genetic diagnosis of insulin resistance phenotypes may be located in the exons, introns, coding region, 5′ UTR region, 3′ UTR region, enhancer region(s) or promoter of the ATF6-alpha gene or locus. In a preferred embodiment, the polymorphisms may be located in the coding region of the ATF6-alpha gene. In another preferred embodiment, the polymorphisms may be located in the 3′ UTR of the ATF6-alpha gene. In another preferred embodiment, the polymorphisms may be located in the promoter of the ATF6-alpha gene. In a further preferred embodiment, the polymorphisms may be located in exon 3, exon 5 or the 3′ UTR of the ATF6-alpha gene or locus. In an even further preferred embodiment, the polymorphism results in an amino acid substitution of the ATF6-alpha gene or locus as shown in SEQ ID NO: 8, illustrated in FIG. 4.

Some polymorphisms may influence the expression of a particular gene and/or the metabolism, structure and/or function of the gene products, including the primary RNA transcript, the mRNA and/or the encoded protein. Such polymorphisms are commonly termed “functional”. Functional polymorphisms may typically be located within the gene or close to the gene which they influence. By way of example and not limitation, functional polymorphisms may influence the transcription of a gene, e.g., the chromatin organization, the levels, the regulatory control and/or the temporal and spatial pattern of transcription. Alterations in transcription may likely alter production of the corresponding protein. Functional polymorphisms may also affect the structure of the corresponding mRNA, e.g., affect the use of alternative promoters or lead to differential splicing of exons during processing of the primary transcript. Further, some functional polymorphisms may alter the metabolism and/or function of the mRNA, e.g., its nuclear export, localization, polyadenylation, stability, turnover and/or efficiency of translation. Yet other functional polymorphisms may result in changes in the amino acid sequence of the corresponding protein (e.g., in insertion, deletion or substitution of one or more amino acids, or truncation of the protein) and may thereby affect the structure, metabolism and/or function of the protein. For example, such polymorphisms may influence the folding, stability, turnover or cellular localization of the protein or its further functional aspects. Other aspects particularly relevant to ATF6-alpha protein may include, e.g., detection of ER stress, translocation to and cleavage in the Golgi apparatus, translocation of the soluble N-terminal fragment to the nucleus, interaction of the soluble N-terminal fragment with its binding partners and/or activation of expression of the target genes. Accordingly, in an embodiment, the polymorphisms in the ATF6-alpha gene or locus for use in the present method of genetic diagnosis of insulin resistance phenotypes may be functional polymorphisms.

Other polymorphisms do not alter the expression of any gene, nor the metabolism, structure or function of any gene products, including the primary RNA transcripts, the mRNAs and the encoded proteins. Such polymorphisms are commonly termed “silent”. Typically, polymorphisms located in intergenic regions, i.e., chromosomal regions interposed between genes, may be silent. However, also many polymorphisms located in genes may be silent. By way of example and not limitation, polymorphisms located in introns of a gene do not affect the amino acid sequence of the protein encoded by that gene. Moreover, many intronic polymorphisms will not have any effect on the splicing and/or regulatory signals located within the introns. Similarly, due to the degeneracy of the genetic code, polymorphisms in coding regions of a gene will not always lead to an amino acid change in the corresponding protein. Further, polymorphisms in the coding region may alter amino acids which are not essential for the structure, metabolism and function of the protein, or may replace an amino acid with a chemically similar amino acid which does not alter the structure, metabolism and function of the protein. Accordingly, in an embodiment, the polymorphisms in the ATF6-alpha gene or locus for use in the present method of genetic diagnosis of insulin resistance phenotypes may be silent polymorphisms.

Still other polymorphisms may be silent in some circumstances, but may become manifest functional polymorphisms in other conditions.

In genetic nomenclature the term polymorphism was traditionally used for sequence variations occurring in normal population, i.e., in population of individuals not having a given phenotype of interest, such as, for example, a particular disease. On the other hand, the term mutation was traditionally reserved for sequence variations which in homozygous or heterozygous state caused a given phenotype of interest (in humans mostly a disease). Such mutations therefore did not occur in the respective homozygous or heterozygous state in normal population. Such distinction may be relatively easy to make when a mutation causes a monogenetic disease with high penetrability (e.g., more than 90%) and low phenocopy rate (e.g., less than 10%). However, it is more difficult to apply the traditional meanings of polymorphism and mutation in phenotypes, such as diseases, with complex aetiology. For example, a given allele of a functional polymorphism may increase the susceptibility of subjects to develop a particular disease, thus resembling a traditional mutation, yet said allele may be frequently found in individuals from normal population, thus resembling a traditional polymorphism. Therefore, the term “sequence variation” is becoming preferred to encompass any sequence change in a genome, regardless of its classification as a polymorphism or as a mutation according to the traditional criteria.

Importantly, as already stated above, the term “polymorphism” as used herein refers generally to the occurrence of two or more alternative nucleotide sequences at a given site within a genome between individuals in a population. Hence, the term “polymorphism” as used herein encompasses any sequence variation at a given site within the genome, regardless of the frequency of the individual allelic forms in any population and regardless of any possible effects of any of the allelic forms on any phenotypic trait.

Polymorphisms with a known position in the genome were historically useful in genetic mapping strategies and therefore may be often referred to as “genetic markers”, “polymorphic markers” or “markers”. Herein, these terms may be used interchangeably with “polymorphism”.

The term “gene” refers to a nucleic acid (e.g., DNA) sequence that comprises coding sequences necessary for the production of a polypeptide (e.g., ATF6-alpha protein) and/or RNA (e.g., ATF6-alpha RNA or mRNA). The polypeptide can be encoded by a full-length coding sequence or by any portion of the coding sequence so long as the desired activity or functional properties (e.g., biological activity, such as enzymatic activity, ligand binding, signal transduction, immunogenicity, etc.) of the full-length polypeptide are retained in the fragment. The term also encompasses the coding region of a structural gene (i.e., a gene coding for a polypeptide) and the sequences located adjacent to the coding region on both the 5′ and 3′ ends for a distance of about 1 kb or more on either end such that the gene corresponds to the length of the full-length mRNA. Sequences located 5′ (or upstream) of the coding region and present in the mRNA are referred to as 5′ untranslated region (5′ UTR). Sequences located 3′ (or downstream) of the coding region and present in the mRNA are referred to as 3′ untranslated region. The term “gene” encompasses both cDNA and genomic forms of a gene. A genomic form or a genomic clone of a gene contains the coding region interrupted with non-coding sequences termed “introns” or “intervening regions” or “intervening sequences”. Introns are segments of a gene that are transcribed into nuclear RNA (also known as heteronuclear RNA, hnRNA or primary transcript); introns may contain regulatory elements, such as enhancers. Introns are removed or “spliced out” from the nuclear RNA; introns are therefore absent in the messenger RNA (mRNA) transcript. The mRNA functions during translation to specify the sequence or order of amino acids in a nascent polypeptide. A genomic form of a gene also encompasses regulatory elements, such as one or more promoters which may direct transcription of the gene. A promoter may often be located in a region (e.g., 1 to 10 kb) upstream of the sequence encoding the 5′ most portion of the RNA or mRNA. A single gene may specify one or more than one transcription unit, i.e., polynucleotide from which RNA may be transcribed; alternative promoters may be used for the different transcripts. Further, an hnRNA transcript may be processed, e.g., spliced (alternative splicing), in different ways to produce more than one different mRNA species; these alternative mRNAs may encode different variants of the corresponding protein. Heteronuclear RNA, mRNA, nascent polypeptide and mature proteins produced from a gene are collectively referred to herein as “gene products”.

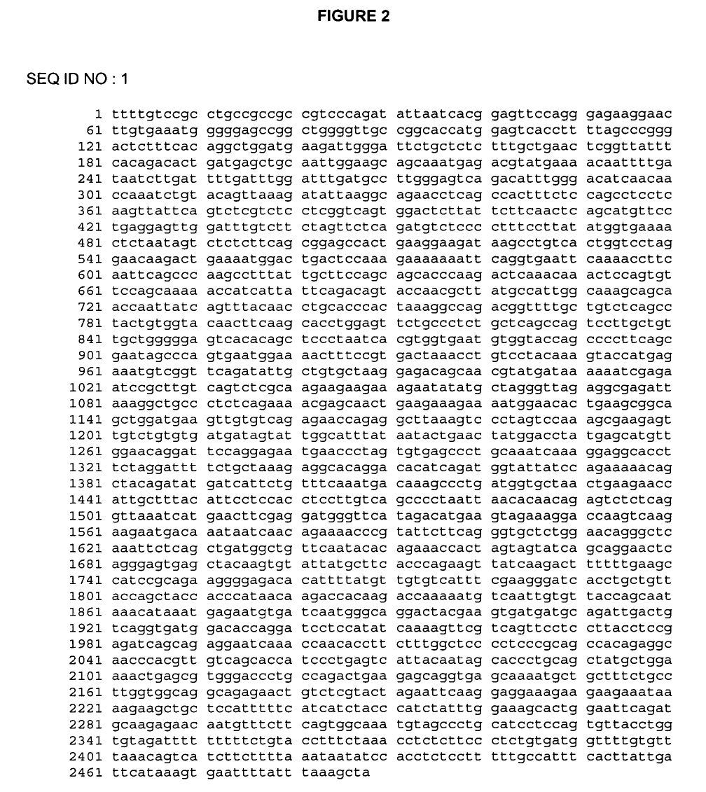

The information contained herein about the genomic, mRNA and cDNA forms of the human ATF6-alpha gene is derived from the entry in the NCBI Entrez Gene database (www.ncbi.nlm.nih.gov/entrez/) under Gene ID: 22926, which is incorporated herein by reference. The nucleic acid sequence as shown in SEQ ID NO: 1 (FIG. 2) corresponds to full-length human ATF6-alpha cDNA and is derived from the nucleic acid sequence present in the NCBI database of reference sequences (www.ncbi.nlm.nih.gov/entrez/) under RefSeq ID: NM_{—}007348, which is herein incorporated by reference. The nucleic acid sequence as shown in SEQ ID NO: 1 (FIG. 2) comprises a coding region from nucleotide 68 to nucleotide 2080, which encodes the full-length human ATF6-alpha protein, the amino acid sequence of which is found in the NCBI database of reference sequences under RefSeq ID: NP_{—}031374, herein incorporated by reference.

The genomic form of human ATF6-alpha gene comprises a nucleic acid sequence on chromosome 1 to which the cDNA sequence as shown in SEQ ID NO: 1 (FIG. 2) may be mapped. According to NCBI Entrez database (Gene ID: 22926), the genomic form of human ATF6-alpha gene comprises a sequence on chromosome 1 from nucleotide 158,467,768 to nucleotide 158,660,510 from pter of approximately 192 kb. The cDNA sequence as shown in SEQ ID NO: 1 (FIG. 2) maps on the direct (+) strand. This sequence may be accessed, for instance, in Ensembl Human Gene View (http://www.ensembl.org) under Gene ID: ENSG00000118217. The genomic form of the ATF6-alpha gene is organized into 16 exons which comprise the sequences present in mRNA and cDNA and 15 introns. In addition to this nucleic acid sequence, the genomic form of human ATF6-alpha gene may further comprise up to 10 kb flanking the above nucleic acid sequence on both 5′ and 3′ sides. These flanking sequences may comprise regulatory elements controlling transcription of the ATF6-alpha gene, e.g., promoter(s) or enhancers.

The sequence of the ATF6-alpha gene, including its genomic and cDNA forms, and the sequence of the ATF6-alpha protein, may differ between subjects in a population and may also differ between the homologous chromosomes in one subject. For example, the ATF6-alpha cDNA sequence as shown in SEQ ID NO: 1 (FIG. 2) represents a reference sequence from which the ATF6-cDNA sequences present in subjects in a population may differ at one or more nucleotide positions. The same applies to other sequences of the ATF6-alpha gene (genomic or cDNA) and ATF6-alpha protein present in databases. The ATF6-alpha gene is therefore intended to include all variations in the nucleic acid sequence, particularly such variation as found in population.

The term “locus” as used herein refers generally to a unique site on a chromosome. A “locus” may be defined by a single nucleotide pair within a chromosome or by a segment of two or more consecutive nucleotide pairs within said chromosome. When used in relation to a gene, the term “locus” denotes a region within a chromosome which comprises a particular gene.

Accordingly, the term “ATF6-alpha locus” as used herein refers to a region within a chromosome which comprises the nucleotide sequence of the ATF6-alpha gene. The ATF6-alpha locus is intended to encompass the transcribed sequences of the ATF6-alpha gene, including the coding sequences, the exons and the introns, as well as regulatory elements controlling the transcription and/or translation of the ATF6-alpha gene. In humans, the ATF6-alpha locus is found on chromosome 1q22-q23 (according to NCBI, www.ncbi.nlm.nih.gov), or more particularly 1q23.3 (according to Ensembl genome browser, www.ensembl.orq). The ATF6-alpha locus on human chromosome 1q22-q23 comprises at least the sequence to which the ATF6-alpha cDNA may be mapped, and more preferably at least 5 kb, or at least 10 kb, or at least 50 kb of genomic sequence on each side of the above sequence, in where the regulatory elements of the ATF6-alpha gene may be present. Therefore, according to NCBI Genbank (www.ncbi.nlm.nih.gov/entrez/), the ATF6-alpha locus on human chromosome 1 may comprise at least the sequence between nucleotide 158,467,768 to nucleotide 158,660,510 from pter, and preferably at least 5 kb, or at least 10 kb, or at least 50 kb extending on each side of this sequence. For example, the ATF6-alpha locus may represent the entire region on chromosome 1 interposed between the genes flanking the ATF6-alpha gene. According to NCBI Genbank, these flanking genes are the gene for dual specificity phosphatase 12, DUSP12, Gene ID: 11266 (nucleotides 158451239 to 158458610) and the gene for olfactomedin-like 2B, OLFML2B, Gene ID: 25903 (nucleotides 158725302 to 158684641 from pter). Accordingly, the human ATF6-alpha locus may correspond to the region on chromosome 1 between nucleotide 158458610 and nucleotide 158725302, of approximately 267 kb. In another example, the International Radiation Hybrid Mapping Consortium (www.ncbi.nim.nih.gov/genemap/page.cqi?F=Consortium.html; Deloukas et al. Science 282: 744-746, 1998) mapped the ATF6-alpha gene using Genebridge 4 and Genebridge 3 radiation hybrid panels on human chromosome 1 in the region between genetic markers D1S2705 (175.1 cM) and D1S2768 (176.8 cM). Accordingly, in the broadest sense, ATF6-alpha locus may encompass the region on chromosome 1 delimited by these markers. The ATF6-alpha locus is intended to include all variations in the DNA sequence as found in population.

The term “nucleic acid” refers to any nucleic acid containing molecule, including but not limited to, DNA, cDNA and RNA.

In an embodiment of the present method of genetic diagnosis, said at least one polymorphism in the ATF6-alpha gene or locus shows genetic association with one or more insulin resistance phenotypes in at least one population.

In another embodiment of the present method of genetic diagnosis, said at least one polymorphism in the ATF6-alpha gene or locus belongs to a group of two or more polymorphisms which shows genetic association with one or more insulin resistance phenotypes in at least one population. Preferably, said group of two or more polymorphisms belongs to a single haplotype block in said at least one population. It is well known in the art that chromosomes comprise sizeable regions, called “haplotype blocks”, which tend to be inherited without recombination (Gabriel et al. Science 296: 2225-2229, 2002). A given haplotype block may typically comprise a number of polymorphic loci. Due to the relative absence of historical recombination within haplotype blocks, particular alleles of polymorphisms located within a given haplotype block tend to be inherited together. As a result, a definite number of haplotypes, i.e., particular combinations of alleles at the different polymorphic loci, may exist in a population for a given haplotype block. Moreover, of these haplotypes, usually only a few (e.g., 2 to 4) common ones may account for a large proportion (e.g., 90% or more) of chromosomes in the population. The polymorphisms within a given haplotype block therefore form a group which may show genetic association with a particular phenotype of interest.

Further, when haplotypes for a given haplotype block need to be determined in a subject, it may often not be needed to determine the genotype of the subject for all polymorphic loci within said haplotype block. For example, it is known in the art that often only a subset of polymorphisms from a given haplotype block can be selected, wherein the genotype of a subject for this subset of polymorphisms will define the haplotypes for said haplotype block present in the subject. Polymorphisms which belong to such subsets are known as “haplotype tagging” polymorphisms (Johnson G C et al., Nat Genet. 29: 233-237, 2001). Accordingly, in an embodiment of the present method of genetic diagnosis, said at least one polymorphism in the ATF6-alpha gene or locus is a haplotype tagging polymorphism. Preferably, said at least one polymorphism will be a haplotype tagging polymorphism for a group of polymorphisms which belong to a single haplotype block, wherein said group of polymorphisms shows genetic association with one or more insulin resistance phenotypes. A skilled person will appreciate that one or more than one such haplotype tagging polymorphisms need to be tested in a subject in order to define the haplotypes for said group of polymorphisms present in said subject.

Boundaries of haplotype blocks need not correspond to boundaries of individual genes. Accordingly, in a given population there may exist one or more haplotype blocks which are entirely located within the ATF6-alpha gene or locus; or there may exist a single haplotype block encompassing the entire ATF6-alpha gene or locus; or there may exist one or more haplotype blocks which comprise at least a portion of the ATF6-alpha gene or locus, as well as sequences outside of the ATF6-alpha gene or locus.

Accordingly, haplotype blocks may exist which comprise only polymorphisms located in the ATF6-alpha gene or locus. Alternatively, haplotype blocks may exist which comprise polymorphisms located in the ATF6-alpha gene or locus, as well as polymorphisms located outside of the ATF6-alpha gene or locus. Groups of polymorphisms from any of such haplotype blocks may show genetic association with one or more insulin resistance phenotypes. Therefore, a skilled person will understand that polymorphisms which are not located in the ATF6-alpha gene or locus, but belong to a haplotype block which comprises at least one polymorphism from the ATF6-alpha gene or locus, may also be useful in the present method of genetic diagnosis. Such polymorphisms may be tested in a subject in the present method of genetic diagnosis. Such polymorphisms may in particular be tested if they are haplotype tagging polymorphisms for said haplotype blocks.

Polymorphisms useful in the present method of genetic diagnosis may preferably show genetic association with one or more insulin resistance phenotype and/or may belong to a group of two or more polymorphisms which shows genetic association with one or more insulin resistance phenotype, wherein said group of polymorphisms may preferably belong to a single haplotype block.

As used herein, a polymorphism or a group of two or more polymorphisms shows “genetic association” with one or more insulin resistance phenotypes in a population, when a statistically significant finding of genetic association is realized in at least one genetic association study in at least one population, as known in the art and detailed below.

Polymorphisms or groups of two or more polymorphisms in the ATF6-alpha gene or locus which show genetic association with insulin resistance phenotypes may be identified using genetic association studies known per se. Genetic association studies determine whether the genotype of subjects for a particular polymorphism in the ATF6-alpha gene or locus, or the haplotype of subjects for a particular group of two or more polymorphisms comprising at least one polymorphism from the ATF6-alpha gene or locus, correlates with the likelihood of said subjects to have certain insulin resistance phenotype(s). If such correlation is found, it may be concluded that at least in the tested group of subjects, and generally in the population for which said subjects are representative, the polymorphism or group of polymorphisms shows “genetic association” with said phenotype. When particular allele(s) and/or genotype(s) of said polymorphism or particular haplotype(s) of said group of polymorphisms are more or less prevalent in subjects having the phenotype than in subjects not having the phenotype, it may be concluded that such allele(s) and/or genotype(s) and/or haplotype(s) “correlate” or are “associated” with an increased or decreased, respectively, susceptibility of subjects to develop said phenotype.

Genetic association studies may have various designs well known to those skilled in population genetics. For example, genetic association studies may be family-based, such as the Transmission Disequilibrium Test (TDT) developed by Spielman et al. (Am J Hum Genet. 52(3):506-516, 1993). The TDT test uses a collection of isolated nuclear families, each having at least one offspring with the phenotype of interest. The test determines whether one allele of a biallelic polymorphism is preferentially transmitted to said offspring. If such preferential transmission occurs, this indicates that said allele is associated with an increased susceptibility of subjects to develop said phenotype.

The originally described TDT test had several limitations, e.g., it only used biallelic polymorphisms and it required genotypes of both parents. To overcome these and other limitations, several modifications of the TDT test have been subsequently developed, e.g., the “sib-TDT” test (Spielman et al. Am J Hum Genet. 62: 450-8, 1998) which genotypes unaffected (i.e., not having the phenotype) sibs of the affected (i.e., having the phenotype) offspring instead of parents; “extended TDT” (Sham et al. Ann Hum Genet. 59: 323-36, 1995) which allows the use of multiallelic polymorphisms; “pedigree disequilibrium test” (PDT) (Martin et al. Am J Hum Genet. 67: 146-54, 2000) which allows the use of related nuclear families from extended pedigrees, etc. These and other family-based tests and their advantages will be known to a skilled population geneticist. As an exemplary advantage, the sib-TDT test may be particularly suited for late-onset diseases, e.g., type 2 diabetes, in which parent genotypes are often not available.

Alternatively, genetic association studies may be population-based, i.e., using subjects independently collected in a population and not having a discrete familial relationship. A population-based study may ideally be a prospective study, in which a cohort of subjects is sampled at a time when said subjects do not display the phenotype of interest and their developing or not developing of said phenotype is observed over time and correlated with their genotype for particular polymorphisms and/or groups of polymorphisms. However, because prospective studies require large samples of subjects and are time consuming, retrospective study designs may often be preferred. A particularly popular retrospective study design are “case-control” studies in which the association of polymorphisms and/or groups of polymorphisms with a phenotype of interest is investigated by comparing a sample of subjects having said phenotype with a control sample of subjects not having the phenotype.

A typical case-control genetic association study relevant in the present invention may comprise the following steps: (i) providing a “case” sample of subjects having an insulin resistance phenotype, e.g., insulin resistance or a disease of insulin resistance; (ii) providing a “control” sample of subjects not having said phenotype, but being on average comparable to the case sample in other parameters, e.g., ethnic ancestry, gender, age, exposure to relevant environmental risk factors, etc., such that any confounding effects of such parameters are avoided (i.e., avoiding the effects of population stratification); (iii) testing the subjects in said case and control samples for at least one individual polymorphism or for a group of two or more polymorphisms in the ATF6-alpha gene and determining the genotypes or haplotypes, respectively, of said subjects for said individual polymorphism or said group of polymorphisms; (iv) comparing the distributions of alleles and/or genotypes of said at least one individual polymorphism or the distributions of haplotypes for said group of two or more polymorphisms between the case and control samples and determining whether or not a statistically significant difference exists between said distributions.

The latter comparison is typically carried out using the CHI square method well-known to a skilled person. This method tests the ‘null hypothesis’ that there exists no difference between the distributions of alleles and/or genotypes and/or haplotypes between the case and control samples. The CHI square test yields a corresponding CHI square value having an attached statistical probability value (P). If the P-value lies below a certain threshold of significance, conventionally P<0.05, it may be concluded that the null hypothesis has been disproved, i.e., that there does exist a statistically significant difference between said distributions. Accordingly, it may than be concluded that there exists genetic association between the tested polymorphism or group of polymorphisms and the phenotype, at least in the population from which the case and control samples have been collected.

When the tested polymorphism only includes two alleles, i.e., it is “biallelic” (or when only two alleles are observed in the tested case and control groups), it can be readily calculated, in the event of a statistically significant CHI square test, which allele and/or genotype(s) are more prevalent in the case sample than in the control sample. When the tested polymorphism includes more than two alleles, i.e., it is “multiallelic” (and when more than two alleles are observed in the tested case and control groups), the contributions of the individual alleles and/or genotypes to the significance of the global CHI square test must be determined, e.g., by carrying out partial CHI square tests, in which every time one of the alleles or genotypes is not considered and a CHI square value is calculated for the remaining alleles or genotypes. If the P-value yielded by such partial CHI square test is greater (i.e., less significant) than the P-value of the global CHI square test, it may be concluded that the allele or genotype which has not been considered in said partial CHI square test is partially or completely responsible for the observed genetic association between the polymorphism and the phenotype. The above considerations may also be readily applied to analysis of distribution of haplotypes of a group of polymorphisms, as will be understood by a skilled person.

Genetic association studies also provide an estimate of the strength of the association between the particular polymorphism or group of polymorphisms and the phenotype—the “odds ratio” (OR). OR is a factor of multiplication by which the odds of a subject having particular allele(s) or genotype(s) of said polymorphism or haplotype(s) of said group of polymorphisms to develop the phenotype are greater or smaller than the odds of a subject not having said allele(s) or genotype(s) or haplotype(s) to develop the same phenotype. OR>1 indicates increased odds of developing the phenotype when a particular allele and/or genotype(s) or haplotype(s) are present, whereas OR<1 indicates reduced odds of developing the phenotype when the particular allele and/or genotype(s) or haplotype(s) are present. OR of 1 indicates no effect of the presence of the particular allele and/or genotype(s) or haplotype(s) on the odds of developing the phenotype. The OR calculated based on the samples is provided with a 95% confidence interval (CI) which defines a range of values that include the actual population OR with a 95% probability. Generally, if the OR is other than 1, it will only be considered significant if its 95% Cl does not include 1.

When calculating the OR, different hypotheses may be considered. For example, in case of an individual polymorphism, it may be assumed that the presence of one copy of an allele of a polymorphism may be sufficient to increase the odds of a subject to develop the phenotype. Then, OR may be calculated for the cluster of genotypes including at least one such allele, relative to genotypes not having said allele. Alternatively, it may be assumed that the presence of two copies of an allele is necessary to increase the odds of a subject to develop the phenotype. Then, OR may be calculated for the homozygous genotype including two copies of said allele, relative to a cluster of genotypes including only one or no such allele. These consideration as well as details of OR calculation will be apparent to a population geneticist. A population geneticist will also be able to extend these considerations to haplotypes of groups of polymorphisms.

In the present context, genetic association studies may be employed to identify those polymorphisms or groups of polymorphisms in the ATF6-alpha gene or locus which may be useful in genetic diagnosis of susceptibility of subjects to various insulin resistance phenotypes. Essentially all such genetic association studies would probably involve collecting subjects having an insulin resistance phenotype, for which genetic association with one or more individual polymorphisms or with a group of two or more polymorphisms in the ATF6-alpha gene or locus is to be investigated. Many studies will further compare the genotype of said subjects with the genotype of subjects not having said phenotype. Therefore, insulin resistance phenotypes for genetic association studies need to be carefully defined. By way of example and not limitation, such phenotypes may comprise: insulin resistance, as defined by impaired fasting glucose and/or impaired glucose tolerance and/or elevated fasting insulin and/or euglycemic insulin clamp technique; a disease of insulin resistance, comprising insulin resistance syndrome and type 2 diabetes; progression from insulin resistance to a disease of insulin resistance within a given time period; worsening of insulin resistance by at least one given measurable parameter value (e.g., IFG, IGT, etc.) within a given time period; progression of a disease of insulin resistance within a given time period; increased clinical severity of a disease of insulin resistance; and response of insulin resistance or a disease of insulin resistance to a method of prevention or therapy (e.g., dietary changes, physical activity, removing risk factors). Accordingly, if the study uses a sample of control subjects, these subjects will not have the above phenotypes or will clearly differ from the case subjects in these phenotypes.

When performing a genetic association study, it is important to properly select polymorphisms in the ATF6-alpha gene or locus which will be genotyped. Such polymorphisms may include any known polymorphisms listed in the existing databases, such as SNP polymorphisms present in the databases mentioned elsewhere in this specification, or any newly identified polymorphisms. Identification of new polymorphisms may typically be performed in a subset of subjects (e.g., in >10, or >20 subjects) from the samples (usually from the control sample) in which an association study is to be performed.

A plurality of methods to identify novel polymorphisms from a particular gene or locus in a group of subjects are known to a skilled person. For example, detection of new polymorphisms may be accomplished by molecular cloning of nucleic acid segments from the ATF6-alpha gene or locus of subjects, direct sequencing these cloned segments by well-known techniques (e.g., the dideoxy chain termination method using, e.g., fluorescently labelled dideoxynucleotides, or the Maxam-Gilbert method; see Sambrook et al. Molecular Cloning A Laboratory Manual, 2nd ed., CSHP, New York, 1989; high throughput automated sequencing systems, e.g., capillary-based or microchip based, can be used) and comparing the obtained sequences, thereby identifying sequence variations within or between subjects which may represent novel polymorphisms. Alternatively, nucleic acid segments from the ATF6-alpha gene or locus can be amplified using known techniques, such as polymerase chain reaction (PCR), directly from genomic DNA or mRNA of subjects, and these amplification products can be sequenced. Comparing the obtained sequences can identify sequence variations within or between subjects which may represent novel polymorphisms. For a gene as large as ATF6-alpha, sequencing of the entire gene or locus in a plurality of subjects may be labour intensive. It may therefore be preferred to employ techniques which allow detecting the presence of sequence variations in nucleic acid segments amplified from the ATF6-alpha gene or locus in subjects. Once the presence of sequence variation(s) in the amplification product of a given segment of the ATF6-alpha gene or locus has been detected, said amplification product from different subjects may be sequenced to determine the alleles of the polymorphism. A plurality of different nucleic acid segments can be amplified from the ATF6-alpha gene or locus of subjects. It may be desirable that these segments together cover the entire sequence of the ATF6-alpha gene or locus, or at least selected parts thereof, such as exons, or regions present in mRNA (e.g., coding region and/or 5′ UTR and/or 3′ UTR), promoter, etc. Said segments may be amplified from genomic DNA and/or mRNA of subjects. If two or more amplification products are needed to cover a particular portion of the ATF6-alpha gene or locus, such amplification products may preferably be partially overlapping rather than adjacent. The size of the amplified segments may be dictated by limitations of the PCR reaction and/or by the preferred size ranges for use in the methods of polymorphism detection. For example, the size of the amplified segments may range from 100 bp to 2 kb. One approach to detect sequence variations in amplification products is single-stranded conformation polymorphism assay (SSCP) first described by Orita et al. (Proc Natl Acad Sci USA 86: 2766-2770, 1989). Further developments of the method are known to a skilled person. This method detects gel mobility shifts occurring in single-stranded DNA amplification products due to sequence variation(s), e.g., substitution, deletion or insertion of one or more nucleotides, which alter the conformation of the single-stranded DNA. Typically, a segment of a gene or locus (e.g., ATF6-alpha) is amplified in the genetic material from a subject, the amplification product is denatured by heat or chemically and subsequently allowed to refold such that reannealing is prevented and the single-stranded molecules assume a conformation which is at least partially dependent on their sequence. The SSCP method may be typically used for amplification products of up to about 200 bp. The amplification products which do show a mobility shift in SSCP are subsequently sequenced to determine the exact nature of the sequence variation and thereby the alleles of the underlying polymorphism in the nucleic acid segment which has been amplified. Other approaches are based on different mobility of partially denatured double-stranded PCR amplification products the sequence of which differs by one or more basepair, such as denaturing gradient gel electrophoresis (DGGE), as described by Wartell et at. (Nucl Acids Res 18: 2699-2706, 1990) and Sheffield et al. (Proc Natl Acad Sci USA 86:232-236, 1989). Yet other approaches are based on the detection of mismatches (i.e., sites without Watson-Crick base pairing due to, e.g., to deletions, insertions, inversions or substitutions) between otherwise complementary DNA strands. Herein, a given nucleic acid segment is amplified in different individuals, the amplification products are mixed together, denatured and allowed to reanneal. During this reannealing complementary strands from two different PCR products (i.e., amplified from different individuals) may be combined in one double-stranded nucleic acid molecule (which is referred to as a “heteroduplex”; “homoduplex” refers to a double-stranded nucleic acid molecule which is formed by complementary strands of the same PCR product). If the different PCR products had a somewhat different sequence, e.g., they differed in one or more basepairs, such reannealed heteroduplex products will contain mismatches, which can be detected. These approaches are represented by clamped denaturing gel electrophoresis (CDGE) described by Sheffield et al. (Am J Hum Genet. 49: 699-706, 1991), heteroduplex analysis (HA) described by White et al. (Genomics 12: 301-306, 1992), chemical mismatch cleavage (CMC) described by Grompe et al. (Proc Natl Acad Sci USA 86: 5888-5892, 1989) or by methods using proteins which recognize nucleotide mismatches (e.g., E. coli mutS protein) as described by Modrich et al. (Annu Rev Genet. 25: 229-253, 1991). One particularly popular approach based on detection of mismatches in heteroduplexes is denaturing high performance liquid chromatography (DHPLC) described by Underhill et al. (Proc Natl Acad Sci USA 93: 196-200, 1996). Advantageously, PCR products which can be analyzed by DHPLC may be up to about 1 kb long. Hence, DHPLC allows to analyze larger segments of a gene or locus than many other methods.

The polymorphisms for use in a genetic association study may be biallelic or multiallelic.

Preferable polymorphisms for use in a genetic association study may be SNPs.

Preferable polymorphisms for use in a genetic association study may be functional, i.e., having an effect on functional aspects of the ATF6-alpha gene or gene products, e.g., mRNA or protein. In general, it may be advisable to include all potentially functional polymorphisms in a genetic association study. If genetic association is found between a functional polymorphism and a phenotype, it may occur due to a direct effect of the functional polymorphism on the ATF6-alpha gene. This association would not require the existence of linkage disequilibrium and may therefore be easier to detect. Moreover, functional polymorphisms may shed light on the functions of the ATF6-alpha gene or gene products and on cellular pathways involved in disease.

In general, when choosing polymorphisms for use in a genetic association study, one will rarely possess experimental evidence in favour of said polymorphism being functional. Nevertheless, a skilled molecular geneticist can make predictions based on the position and nature of a given polymorphism and about the likelihood of such polymorphism to be functional. For example, polymorphisms which may have a higher chance of being functional include: polymorphisms which cause changes in the amino acid sequence of the ATF6-alpha protein, particularly if such change occurs in a conserved region or domain of the protein; polymorphisms which reside at exon-intron boundaries and which may therefore affect splicing of ATF6-alpha transcripts; polymorphisms in conserved regions or motifs of the ATF6-alpha mRNA, e.g., in 5′ or 3′ untranslated regions, e.g., close to the polyA addition signal, which may influence the efficiency of translation or the stability and/or turnover of mRNA; polymorphisms in the ATF6-alpha promoter, especially in the region of the promoter which binds molecules facilitating the transcription of the ATF6-alpha gene. On the contrary, some polymorphisms may be less likely to be functional, e.g., polymorphisms in the coding sequence which do not change the amino acid sequence; polymorphisms which change an amino acid in an unconserved region or wherein an amino acid is substituted with a chemically similar amino acid; polymorphisms in introns; polymorphisms in unconserved portions of mRNA not containing functional motifs, etc.

A genetic association study may be performed with one or more individual polymorphisms, i.e., polymorphisms for which it is unknown to which haplotype block they belong and with which other polymorphisms they may be in the same haplotype block and/or with which other polymorphisms they may be in linkage disequilibrium.

Preferably, a genetic association study may be performed with one or more groups of two or more polymorphisms, and more preferably, each such group of two or more polymorphisms may belong to a different haplotype block. Importantly, a genetic association study need not be performed using all polymorphism from a given haplotype block, but it may be preferred, e.g., to reduce the cost, to use a subset of haplotype tagging polymorphisms from each haplotype block. Determining the genotype of a subject for such haplotype tagging polymorphisms from a given haplotype block may, at least in most cases, be sufficient to discriminate the (common) haplotypes present in said subject.

To identify haplotype blocks, their respective common haplotypes, and the corresponding haplotype tagging polymorphisms in a chromosomal region of interest, e.g., in a region comprising the ATF6-alpha gene or locus, one may genotype at least a fraction of polymorphisms present in said chromosomal region in a collection of nuclear families. Ideally, all polymorphisms present in said chromosomal region may be genotyped and preferably at least a considerable fraction, e.g., more than 50%, preferably more than 70%, more preferably more than 80%, or most preferably more than 90% of all polymorphisms present in said chromosomal region. Usually, the nuclear families will be selected from the same population as the population in which the genetic association study is to be performed. In the nuclear families it is possible to detect commonly transmitted haplotype blocks and define the common haplotypes and select appropriate haplotype tagging polymorphisms by methods, e.g., computational methods, known in the art. Alternatively, information about haplotype blocks and common haplotypes is also provided by the international HapMap project (www.hapmap.org) which aims at identifying common SNP haplotypes in four populations from different parts of the world and generate identifying “tag” SNP that uniquely identify such haplotypes. Any haplotype block comprising at least one polymorphism from the ATF6-alpha gene or locus may be used in a genetic association study.

These and other considerations will be apparent to a skilled population geneticist planning a genetic association study to identify sequence variations in the ATF6-alpha gene or locus which are linked to an insulin resistance phenotype.

It may be possible that some functional polymorphisms in the ATF6-alpha gene may display characteristics that are usually associated with disease-causing mutations. For example, a particular allele of a given polymorphism may be very rare in a population (e.g., less than 1%, or less than 0.1%, or less than 0.01%) and may be essentially absent in normal subjects from said population. Said allele (“causative” allele) may lead to functional alterations of the ATF6-alpha gene and/or its gene products (ATF6-alpha mRNA or protein) which are drastic enough to cause an insulin resistance phenotype in essentially all or a majority of subjects who are, depending on the mode of inheritance, homozygous or heterozygous for said allele. Hence, the penetration of such allele in either homozygous or heterozygous state may be, e.g., higher than 80%, or higher that 85%, or higher than 90%, or higher than 95% or close to 100%. Genetic association of such polymorphisms with a phenotype may typically not be found in genetic association studies, e.g., because of the causative allele is too rare. Rather, such causative alleles may be found in families in which a particular insulin resistance phenotype segregates in successive generations, by analyzing co-segregation of a particular allele with the phenotype (LOD score>3 in linkage study). Causative alleles may, e.g., lead to non-sense, missense or frame-shift mutations in the ATF6-alpha protein, or may alter the expression of the ATF6-alpha gene or splicing of the transcript, or the stability of mRNA. From the present disclosure, the skilled person will understand that (i) screening of the ATF6-alpha gene or locus for causative mutations may be useful in genetic diagnosis in families segregating an insulin resistance phenotype, and that (ii) so-identified causative mutations will be useful for genetic diagnosis of insulin resistance phenotypes. Therefore, polymorphisms useful in the present method of genetic diagnosis may also encompass causative mutations in the ATF6-alpha gene.

Accordingly, genetic association studies in populations (or alternatively linkage studies in affected families) may identify individual polymorphisms in the ATF6-alpha gene or locus and/or groups of two or more polymorphisms (preferably from a single haplotype block) wherein at least one of the polymorphisms is in the ATF6-alpha gene or locus, which show genetic association in at least one population (or co-segregation of an allele in at least one affected family) with one or more insulin resistance phenotypes. Such polymorphisms may be useful in the present method of genetic diagnosis of insulin resistance phenotypes.

Exemplary insulin resistance phenotypes may include: insulin resistance; a disease of insulin resistance; likelihood and/or rate of progression from insulin resistance to a disease of insulin resistance; rate of progression of insulin resistance; rate of progression of a disease of insulin resistance; severity and/or clinical features of a disease of insulin resistance; response of insulin resistance or of a disease of insulin resistance to a method of treatment. Accordingly, particular allele(s) and/or genotype(s) of said individual polymorphisms in the ATF6-alpha gene or locus and/or particular haplotype(s) of said group of two or more polymorphisms may correlate with an increased or decreased susceptibility of subjects from said at least one population to display and/or develop such phenotypes or phenotypic expressions. For example, particular allele(s) and/or genotype(s) of said at least one polymorphism and/or particular haplotype(s) of said group of two or more polymorphisms may correlate with one or more of: increased or decreased susceptibility of subjects to develop insulin resistance; increased or decreased susceptibility of subjects to develop a disease of insulin resistance; increased or decreased likelihood for progression of subjects from insulin resistance to a disease of insulin resistance; increased or decreased rate of progression of subjects from insulin resistance to a disease of insulin resistance; faster or slower progression of insulin resistance or of a disease of insulin resistance in subjects; increased or decreased severity of a disease of insulin resistance in subjects; and increased or decreased occurrence of particular clinical features in subjects with a disease of insulin resistance; increased or decreased response of subjects to a particular method of treatment. As described, such correlation of allele(s) and/or genotype(s) and/or haplotype(s) with particular phenotypes may be found in genetic association studies.

As described elsewhere, the strength of genetic association between a particular polymorphism or a group of polymorphisms and a phenotype is given by the odds ratio (OR), which represents the factor of multiplication by which the average odds of a subject having particular allele(s) or genotype(s) of said polymorphism or haplotype(s) of said group of polymorphisms to develop said phenotype are greater or smaller than the odds of a subject not having said allele(s) or genotype(s) or haplotype(s) to develop the same phenotype. An OR>1 with a 95% Cl not including 1 for particular allele(s) or genotype(s) or haplotype(s) indicates that the presence of said allele(s) or genotype(s) or haplotype(s) correlates with increased odds of developing the phenotype. An OR<1 with a 95% Cl not including 1 for particular allele(s) or genotype(s) or haplotype(s) indicates that the presence of said allele(s) or genotype(s) or haplotype(s) correlates with decreased odds of developing the phenotype.

Accordingly, detecting the presence or absence in a subject of such allele(s) and/or genotype(s) of such individual polymorphisms and/or such haplotype(s) of such groups of two or more polymorphisms (i.e., testing of a subject for these polymorphisms or groups of polymorphisms) indicates whether the subject may have an increased or decreased odds of developing a given insulin resistance phenotype, such as, by way of example and not limitation: increased or decreased odds to develop insulin resistance; increased or decreased odds to develop a disease of insulin resistance; increased or decreased odds to progress from insulin resistance to a disease of insulin resistance, e.g., within a given time; increased or decreased odds for faster or slower progression of insulin resistance or of a disease of insulin resistance; increased or decreased odds for increased or decreased severity of a disease of insulin resistance; increased or decreased odds for displaying particular clinical features of a disease of insulin resistance; and increased or decreased odds to respond to a particular method of treatment. The OR value then indicates the factor of multiplication by which the odds of the subject are increased or decreased.

Accordingly, individual polymorphisms from the ATF6-alpha gene or locus and/or groups of two or more polymorphisms (preferably from a single haplotype block) wherein at least one of the polymorphisms is in the ATF6-alpha gene or locus for use in the present method of genetic diagnosis may preferably show genetic association in at least one population with one or more insulin resistance phenotypes, and particular allele(s) and/or genotype(s) of said individual polymorphisms or particular haplotype(s) of said groups of two or more polymorphisms may have an OR determined in a genetic association study in at least one sample from said at least one population greater or smaller than 1 with 95% CI not including 1.

Accordingly, in an embodiment, the present method of genetic diagnosis can be employed to determine whether a normal subject may have an increased or decreased susceptibility to develop insulin resistance.

In another embodiment, the present method of genetic diagnosis can be employed to determine whether a normal subject or a subject with insulin resistance may have an increased or decreased susceptibility to develop a disease of insulin resistance.

In further embodiments, the present method of genetic diagnosis may be employed to determine any of the following: whether a subject with insulin resistance susceptible to faster or slower progression of the insulin resistance; whether a subject with insulin resistance is susceptible to faster or slower progression of the insulin resistance; whether a subject with a disease of insulin resistance is susceptible to faster or slower progression of the disease of insulin resistance; whether a subject has an increased or decreased susceptibility to develop a disease of insulin resistance characterised by increased severity and/or presence of particular clinical features; whether a subject with insulin resistance or a disease of insulin resistance has an increased or decreased odds to respond to a particular method of treatment (e.g., one directed to modulating the expression or function of the ATF6-alpha or gene products, e.g., mRNA and protein, or one directed on modulating the related pathways). The subjects to be tested with the present method of genetic diagnosis may thus be normal, may have already developed insulin resistance, or may have already developed a disease of insulin resistance.

Further, the subjects to be tested may also have one or more risk factors for developing insulin resistance and/or diseases of insulin resistance. For example, a well-known risk factor for developing insulin resistance and/or diseases of insulin resistance is obesity, which may be quantified using the body mass index (BMI). Subjects with BMI<25 kg/m² are considered normoweight, subjects with BMI between 25 and 29.9 kg/m² are considered overweight, and subjects with BMI>30 kg/m² are considered obese. Normoweight, overweight and obese subjects may all be tested in the present method of genetic diagnosis. Testing overweight and/or obese subjects may be particularly interesting, as such subjects may already be at risk of developing insulin resistance and/or a disease of insulin resistance. Accordingly, in an embodiment, the tested subjects may have a BMI of 25 kg/m² or more; in another embodiment, the tested subjects may have a BMI between 25 kg/m² and 30 kg/m²; in another embodiment, the tested subjects may have a BMI of 30 kg/m² or more. Moreover, the pattern of obesity is also important, and truncal obesity (i.e., a waist-to-hip ratio more than 0.8 in females and more than 1 in males) is a risk factor for developing insulin resistance and/or a disease of insulin resistance. Accordingly, in an embodiment, the tested subjects may have truncal obesity.

Body Mass Index or BMI is a tool for indicating weight status in adults. BMI is a measure of weight for height. To determine BMI, weight in kilograms is divided by height in meters, squared. For adults over 20 years old, BMI falls into one of these categories:

| BMI | Weight Status |
|---|---|
| Below 18.5 | Underweight |
| 18.5-24.9 | Normal |
| 25.0-29.9 | Overweight |
| 30.0 and Above | Obese |

BMI correlates with body fat. The relation between fatness and BMI differs with age and gender. For example, women are more likely to have a higher percent of body fat than men for the same BMI. On average, older people may have more body fat than younger adults with the same BMI. BMI for Children and Teens is based on gender and age specific charts and is well known by the person skilled in the art. In this regard, reference is made to http://www.cdc.gov/nccdphp/dnpa/bmi/

Other potential risk factors for developing insulin resistance and/or diseases of insulin resistance may include race or ethnicity (e.g., African American, Hispanic American, Native-American), age (e.g., more than 45 years), family history of diabetes, personal history of gestational diabetes, sedentary lifestyle, high carbohydrate diet, low serum HDL (<35) and high serum triglycerides (>250), hypertension, and others. Subjects having these factors may be particularly suitable for the present method of genetic diagnosis.

In general, when the present method of genetic diagnosis uses a polymorphism or a group of polymorphisms in the ATF6-alpha gene or locus which shows genetic association with one or more insulin resistance phenotypes in a given population, then subjects to be tested for said polymorphism or said group of polymorphisms will be preferably derived from the same population. This particularly applies when the tested polymorphisms are not functional, i.e., they are silent, i.e., they do not modify the function of the ATF6-alpha gene or gene products (RNA, mRNA, protein). Such polymorphisms may often show genetic association with a phenotype because they are in linkage disequilibrium (see below) with a functional polymorphism elsewhere in the ATF6-alpha gene. However, linkage disequilibrium blocks and co-segregation of alleles of polymorphisms may differ in different populations. Moreover, said functional polymorphism may not be present in all populations or may have independently appeared in different populations.

When the tested polymorphism is functional, i.e., when it modulates insulin resistance phenotype(s) by directly affecting the function of the ATF6-alpha gene or gene products, then it may be predicted that its effects will be comparable in different populations. Hence, subjects to be tested for such a polymorphism may originate from a different population than the one in which the genetic association has been found. Nevertheless, it may still be beneficial to test the association of such functional polymorphism with the phenotype(s) in every newly used population.

A skilled geneticist will further understand that any polymorphisms (herein referred to as “surrogate” polymorphisms) which are in linkage disequilibrium with polymorphisms and/or groups of polymorphisms that show genetic association with one or more insulin resistance phenotypes (as described in the previous paragraph), may also be used in the present method of genetic diagnosis even while genetic association with said one or more insulin resistance phenotypes has not been tested for such surrogate polymorphisms.

Two polymorphisms (typically closely located on a chromosome, e.g., at a genetic distance of less than 5 cM, or less than 1 cM, or less than 0.1 cM, or less than 0.01 cM) are said to be in “linkage disequilibrium” when the observed frequencies of haplotypes (i.e., combinations of alleles of the two polymorphisms present on the same chromosome) in a population differ from the expected frequencies of the haplotypes. Expected frequency for each haplotype may be calculated by multiplying the population frequencies of the individual alleles forming the haplotype. In other words, LD between two polymorphisms represents a situation in which the haplotypes at the two polymorphic loci have not been randomized by historical recombination between the loci. If a particular haplotype is more frequent in the population than would be expected by multiplying the population frequencies of the two alleles forming the haplotype, then said two alleles can also be said to be in LD.

In the present method of genetic diagnosis, the effect of LD may be exemplified as follows: if a given polymorphism in the ATF6-alpha gene or locus shows genetic association with an insulin resistance phenotype, and a particular allele of said polymorphism correlates with increased odds of subjects to develop said phenotype, then a particular allele of a surrogate polymorphism which is in LD with said allele of the polymorphism showing genetic association, may also correlate with increased odds of subjects to develop said phenotype. This is because said alleles co-occur in a haplotype more often than would be expected by chance. A skilled geneticist will appreciate this point; he will also understand that in many studies a genetic association may be detected for a silent polymorphism due to the fact that it is in LD with an often unknown functional polymorphism which directly modulates the phenotype. The above example is non-limiting and a skilled geneticist is able to realize the possibilities of using surrogate polymorphisms in the present method of genetic diagnosis. Further, a skilled person will also appreciate that surrogate polymorphisms showing LD with polymorphisms in the ATF6-alpha gene or locus may be used in the present method of genetic diagnosis even if themselves they are located outside of the ATF6-alpha gene or locus as defined herein.

As detailed in example 2, the present inventors found a significant genetic association between the RS13401 SNP polymorphism in the 3′ UTR of the ATF6-alpha gene and insulin resistance (as evidenced by impaired fasting glucose or impaired glucose tolerance) and type 2 diabetes. RS13401 SNP is a single nucleotide substitution polymorphism having two known alleles, G and A, which may be present at position corresponding to nucleotide 2204 in the ATF6-alpha cDNA sequence as show in SEQ ID NO: 1 (FIG. 2). The sequence shown in SEQ ID NO: 1 contains guanine (G) at this position, thereby corresponding to the “G” allele of RS13401. The “A” allele of RS13401 will contain adenine (A) at this position. Further information on the RS13401 SNP is available in the NCBI dbSNP database under the refSNP ID: rs13401 (http://www.ncbi.nlm.nih.gov/proiects/SNP/). Because the RS13401 SNP is transcribed and present in the ATF6-alpha mRNA, it may be analyzed in subjects either in genomic DNA or in mRNA or cDNA. The present inventors found (example 2) a statistically significant correlation between the presence of at least one G allele in the genetic material of subjects and the susceptibility of said subjects to develop and/or display an insulin resistance phenotype.

Accordingly, in an exemplary embodiment, the present invention relates to a method of genetic diagnosis of insulin resistance phenotypes which comprises testing of a sample from a subject for the RS13401 SNP polymorphism in the ATF6-alpha gene, which corresponds to an A/G single nucleotide substitution at a position corresponding to nucleotide 2204 of the ATF6-alpha cDNA sequence as shown in SEQ ID NO: 1 (FIG. 2). In particular, said insulin resistance phenotypes may comprise insulin resistance and type 2 diabetes. In particular, said method may detect the presence in the subject of at least one G allele, or at least one A allele, or the method may determine the genotype (i.e., AA or AG or GG) of the subject for the RS13401 SNP polymorphism. The presence of at least one G allele (i.e., genotypes GA or GG) in a normal subject will indicate that the subject has an increased odds of developing insulin resistance and/or type 2 diabetes compared to a normal subject not having any G allele (i.e., a subject homozygous for the AA allele). For example, the odds may be increased by factor corresponding to the OR found in example 2, i.e., 1.44 with a 95% Cl of 1.09-1.99. The observed value of OR may vary differ in other genetic association studies performed in samples from the same or different populations.

In another embodiment, the method of genetic diagnosis of insulin resistance phenotypes may comprise testing of a subject for a polymorphism which is in linkage disequilibrium with the RS13401 SNP polymorphism in the ATF6-alpha gene, i.e., a surrogate polymorphism for the RS13401 SNP polymorphism.

As detailed in example 5, the present inventors also found genetic association between the RS2070150 SNP polymorphism in the coding region of the ATF6-alpha gene and Familial Combined Hyperlipidemia (FCHL) in females. RS2070150 SNP is a single nucleotide substitution polymorphism having two known alleles, C and G, which may be present at position corresponding to nucleotide 500 in the ATF6-alpha cDNA sequence as show in SEQ ID NO: 1 (FIG. 2). C to G substitution at RS2070150 polymorphic site leads to a Pro to Ala change of amino acid 145 of the ATF6-alpha protein. The sequence shown in SEQ ID NO: 1 (FIG. 2) contains guanine (G) at this position, thereby corresponding to the “G” allele of RS2070150. The “C” allele of RS2070150 will contain cytosine (C) at this position. Further information on the RS2070150 SNP is available in the NCBI dbSNP database under the refSNP ID: rs2070150 (http://www.ncbi.nlm.nih.gov/proiects/SNP/). Because the RS2070150 SNP is transcribed and present in the ATF6-alpha mRNA, it may be analyzed in subjects either in genomic DNA or in mRNA or cDNA. The present inventors found (example 5) a correlation between the presence of the GG genotype of RS2070150 SNP in the genetic material of female subjects and the susceptibility of said female subjects to develop and/or display FCHL.

Accordingly, in an exemplary embodiment, the present invention relates to a method of genetic diagnosis of insulin resistance phenotypes which comprises testing of a subject for the RS2070150 SNP polymorphism in the ATF6-alpha gene, which corresponds to an C/G single nucleotide substitution at a position corresponding to nucleotide 500 of the ATF6-alpha cDNA sequence as shown in SEQ ID NO: 1 (FIG. 2). In particular, said insulin resistance phenotypes include Familial Combined Hyperlipidemia (FCHL) in females. In particular, said method may detect the presence in the subject of at least one G allele, or at least one C allele, and preferably the method may determine the genotype (i.e., GG or GC or CC) of the subject for the RS2070150 SNP polymorphism. The presence of the GG genotype in a normal female subject will indicate that the subject has increased odds of developing FCHL compared to a normal subject not having the GG genotype (i.e., a subject having GC or CC genotypes).

In another embodiment, the method of genetic diagnosis of insulin resistance phenotypes (e.g., FCHL in females) may comprise testing of a subject for a polymorphism which is in linkage disequilibrium with the RS2070150 SNP polymorphism in the ATF6-alpha gene, i.e., a surrogate polymorphism for the RS2070150 SNP polymorphism.

Accordingly, the present invention relates to a method of genetic diagnosis of insulin resistance phenotypes, which comprises testing of a subject for an A/G or an A/T substitution in the ATF6-alpha gene at a position corresponding to nucleotide 266 in ATF6-alpha cDNA sequence as shown in SEQ ID NO: 1 illustrated in FIG. 2 or a C/T substitution in the ATF6-alpha gene at a position corresponding to nucleotide 536 in ATF6-alpha cDNA sequence as shown in SEQ ID NO: 1 illustrated in FIG. 2.

In another embodiment, the method of genetic diagnosis of insulin resistance phenotypes may comprise testing of a subject for at least one known polymorphism in the ATF6-alpha gene or locus, provided that genetic association is found for said polymorphism in at least one population. For example, a skilled person will be able to find known polymorphisms in the ATF6-alpha gene or locus in databases, particular SNP databases, such as listed elsewhere.

The present method of genetic diagnosis involves testing of a sample of a subject for at least one polymorphism in the ATF6-alpha gene or locus, i.e., detecting the presence or absence of particular allele(s) of said at least one polymorphism in the genetic material of the subject and/or determining one or both alleles of said at least one polymorphism in the genetic material of the subject. This may be performed by any suitable method of genotyping as known in the art. As used herein, “genotyping” refers to any method of evaluating genetic material. For example, genotyping encompasses methods of determining the identity of one or more nucleotides (in consecutive or non-consecutive positions), determining the sequence of a segment of nucleic acid, and determining the identity and number of alleles of one or more polymorphisms present in the genetic material from a subject, e.g., genomic DNA, RNA, e.g., total RNA and preferably mRNA, or cDNA prepared from said RNA (e.g., from total RNA or preferably mRNA). Obviously, genotyping is also needed in genetic association studies, wherein the genotype (i.e., both alleles) of one or more polymorphisms needs to be determined in the subjects. Accordingly, in an embodiment, the present method of genetic diagnosis of insulin resistance phenotypes comprises testing of at least one polymorphism in the ATF6-alpha gene or locus in DNA, or RNA, preferably mRNA, derived from a subject or in cDNA prepared from said RNA or mRNA.

In a further embodiment, the present invention relates to a method for genetic diagnosis of an insulin resistance phenotype in a subject for at least one polymorphism in the gene or locus for activating transcription factor 6 alpha (ATF6-alpha), comprising: (i) contacting an oligonucleotide with a sample from said subject under conditions allowing specific hybridization of said oligonucleotide to a nucleic acid in said sample, wherein said nucleic acid comprises at least part of the gene or locus for ATF6-alpha; (ii) determining specific hybridization between said oligonucleotide and said nucleic acid, whereby an insulin resistance phenotype in a subject is diagnosed, wherein said insulin resistance phenotype is chosen from the group comprising insulin resistance syndrome (IRS), type 2 diabetes, metabolic syndrome, familial combined hyperlipidemia (FCHL), familial hyper-triglyceridemia, dyslipidemia, hepatic steatosis, obesity, polycystic ovary syndrome (PCOS), primary diabetes mellitus (DM), hypertension, lipodystrophy, hypercholesterolemia, inflammation and Cushing's syndrome.

The genetic material of a subject may be present in a biological sample collected from said subject, which may contain nucleic acid material including genomic DNA and/or total RNA and/or mRNA. Suitable biological samples may include whole blood, serum, saliva, ejaculated semen, sweat, tears, skin, buccal smears, expectorated sputum, cerebrospinal fluid, urine and faecal material, hair, biopsies of specific organ tissues, such as muscle or adipose tissue, etc. When the biological sample should contain ATF6-alpha mRNA, it needs to be obtained from a tissue or organ in which the ATF6-alpha gene is expressed. The biological sample can be freshly collected or suitably stored to preserve the DNA and/or total RNA and/or mRNA. DNA and/or mRNA may be isolated from the biological sample using methods known in the art, such as methods based on phenol-chloroform extraction or methods employing ion-exchange resins. mRNA may be typically converted to cDNA prior to genotyping. It will be understood by the skilled person that mRNA or cDNA preparations would not be used to detect polymorphisms located in promoters, introns or in 5′ and 3′ nontranscribed regions of the ATF6-alpha gene.

Accordingly, the present invention relates to a method as described herein, wherein said polymorphism is tested in nucleic acid, such as DNA or RNA, preferably mRNA, or in cDNA prepared from said RNA or mRNA.

Useful genotyping methods to test particular polymorphisms of interest in the ATF6-alpha gene or locus of subjects include, but are not limited to: direct sequencing of nucleic acid segments amplified, e.g., by PCR, and/or cloned from the genetic material (genomic DNA and/or mRNA) of a subject; also methods described elsewhere in this specification may be used to test particular polymorphisms of interest in the ATF6-alpha gene or locus, including single strand conformational polymorphism assay (SSCP), denaturing gradient gel electrophoresis (DGGE), clamped denaturing gel electrophoresis (CDGE), heteroduplex analysis (HA), chemical mismatch cleavage (CMC), enzymatic binding of mismatches and denaturing high performance liquid chromatography; further exemplary methods to test particular polymorphisms of interest in the ATF6-alpha gene or locus include PCR-restriction fragment length polymorphism assays, allele-specific amplification assays, detection using allele-specific oligonucleotides, single-base extension assays and oligonucleotide ligation assays, as described in the following. These genotyping methods are typically performed either directly on the genetic material of subjects or optionally on nucleic acid segments of the ATF6-alpha gene or locus which comprise one or more polymorphisms of interest, said segments having been amplified from the genetic material of subjects (genomic DNA, mRNA or cDNA) using a suitable amplification method, most commonly the polymerase chain reaction (PCR). The size of such amplified segments may be dictated by the limitations of the amplification method (PCR) and/or by the preferred segment sizes for use in the detection methods. For example, the size of such amplified segments may typically range from 80 bp to 500 bp, or may be more than 500 bp.

PCR-restriction fragment length polymorphism (PCR-RFLP) can be used when a particular allele of a polymorphism contributes to a recognition site for a restriction endonuclease, while other alleles of said polymorphism destroy this recognition site. Here, a nucleic acid segment from the ATF6-alpha gene or locus comprising the polymorphism is amplified by PCR from the genetic material (DNA and/or mRNA) of a subject, the amplification product is subjected to cleavage by said restriction endonuclease, and resolved by gel electrophoresis. If cleavage fragments having a smaller size than the PCR product are observed, this indicates that the allele that contributes to the restriction site is present in the subject. If only cleaved fragments but no uncleaved PCR product is observed, then the subject is likely homozygous for said allele. If both the cleaved fragments and uncleaved PCR product are observed, the subject may be heterozygous having said allele and another allele. If only the uncleaved PCR product is observed, the subject likely does not contain said allele. For biallelic polymorphisms, PCR-RFLP can thus determine the genotype of the subject.

Allele-specific amplification (ASA) has been introduced by Wu et al. (Proc Natl Acad Sci USA 86: 2757-60, 1989). Two or more first oligonucleotide primers, each specific for nucleic acid comprising only a particular allele of a polymorphism from the ATF6-alpha gene or locus, are used in conjunction with a second primer common to nucleic acids comprising any of the alleles of said polymorphism in separate polymerase chain reactions (PCR) using the genetic material of a subject (e.g., genomic DNA, mRNA, cDNA). The allele-specific first primers usually differ from each other in their terminal 3′ nucleotide (this position is most destabilizing to elongation from the primers), wherein said terminal 3′ nucleotide of each first primer is complementary to a different allele of the polymorphism. Therefore, each first primer will, under properly selected annealing temperature and PCR conditions, only prime synthesis from a nucleic acid comprising the allele to which it is complementary, but not from nucleic acids comprising other alleles of the polymorphism. The second primer will by typically complementary to a sequence distal to the polymorphic site and therefore will anneal to and prime synthesis from nucleic acids comprising any allele of the polymorphism. Therefore, each first primer in conjunction with the second primer will only amplify (i.e., produce a detectable amplification product) the target sequence in a nucleic acid molecule comprising a particular allele of the polymorphism, thereby detecting the presence (or absence) of said allele in the genetic material of the subject. ASA may be particularly suited for the detection of SNP polymorphisms, such as, e.g., substitution SNPs. It is also contemplated that allele-specific amplification can be performed simultaneously at different polymorphic sites in the same PCR reaction, provided that the amplification products from the different polymorphic sites differ in their sizes or are otherwise distinguishable. An analogous technique of polymorphism detection, called amplification refractory mutation system (ARMS) and disclosed in EP 0332435 and in Newton et al. (Nucleic Acids Res 17: 2503-2516, 1989), may also be useful herein.

A polymorphism may also be tested in a segment of the ATF6-alpha gene or locus, either directly in the genetic material of a subject or after amplification from the genetic material (DNA and/or mRNA) of a subject by PCR, using allele-specific oligonucleotide (ASO) probes. As used herein, the term “probe” refers to a double-stranded or single-stranded oligonucleotide or polynucleotide, typically labelled, which anneals to and forms a stable hybrid in a nucleic acid hybridization reaction with a target nucleic acid due to complementary base pairing under stringent to moderately stringent hybridization and wash conditions. Hybridization and wash conditions are chosen to rule out nonspecific and adventitious annealing, i.e., to minimize noise. The probe will comprise a hybridizing region, preferably consisting of 10 to 50 nucleotides, more preferably 20 to 30 nucleotides, corresponding to a region of the target sequence. The hybridizing region of a probe is preferably identical or fully complementary to the sequence of the target region. The hybridizing region may also contain a certain number of mismatches, those skilled in the art of nucleic acid technology can determine duplex stability considering a number of variables including the length and base-pair composition of the probe, ionic strength of the buffer, reaction temperature and incidence of mismatched base pairs. The design and use of allele-specific oligonucleotide probes for analyzing polymorphisms is disclosed in, for example, Saiki et al. (Nature 324: 163-166, 1986), EP 0235726, and WO 89/11548. ASO probes can be designed that will be completely complementary to a nucleic acid comprising a particular allele of a polymorphism form the ATF6-alpha gene or locus, but will show a mismatch of at least one base pair at said polymorphic site when annealed to a nucleic acid comprising a different allele of said polymorphism. Stringent hybridization conditions can be used under which an ASO probe will specifically hybridize only to nucleic acids to which it shows completely complementarity, such as to nucleic acids comprising a particular allele of said polymorphism, but will not bind substantially to nucleic acids with which it shows one or more mismatches, such as to nucleic acids comprising other alleles of said polymorphism. Therefore, specific hybridization of an ASO probe to the genetic material of a subject or to a PCR product from a subject will indicate the presence in said subject of at least one allele of the polymorphism which is recognized by the ASO probe. The ASO probe will carry a suitable label, such as an enzymatic, fluorescent or radioactive label. Hybridization conditions should be sufficiently stringent that there is a significant difference in hybridization intensity between alleles, and preferably an essentially binary response, whereby an ASO probe hybridizes to only one of the alleles. Hybridizations may be usually performed at conditions, for example, of salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C., or equivalent conditions, are suitable for ASO probe hybridizations. Equivalent conditions can be determined by varying one or more of the parameters given as an example, as known in the art. Some ASO probes are designed to hybridize to a segment of target DNA such that the polymorphic site aligns with a central position (e.g., in a 15-mer at the 7 position; in a 16-mer, at either the 8 or 9 position) of the probe. This design of probe achieves good discrimination in hybridization between different allelic forms. ASO probes are often used in sets, one member of a set showing perfect complementarity to the reference allelic form of a target sequence and the other members showing perfect complementarity to the variant allelic forms. Several sets of ASO probes can then be immobilized on the same solid support for simultaneous analysis of multiple polymorphisms within the same target sequence. In the latter case the sample hybridized to so-immobilized ASO probes will be labelled, e.g., using a fluorescent label. Further, the same solid support may also comprise ASO probe sets for one or more other polymorphisms from the ATF6-alpha gene or locus or from other genes or loci. This arrangement will essentially correspond to an array of ASO probes, e.g., a microarray. Allele-specific oligonucleotide probes can comprise DNA, peptide nucleic acid (PNA) and RNA, or combinations thereof. ASO probes may be, e.g., 15-30 nucleotides in length.

Further, a polymorphism may also be tested in a segment of the ATF6-alpha gene or locus, which has been amplified from the genetic material (DNA and/or mRNA) of a subject by PCR, by single-base extension (SBE) assays. Here, a primer is designed which hybridizes adjacent to a particular polymorphic site of interest from the ATF6-alpha gene or locus in said amplification product, whereby the terminal 3′ nucleotide of the primer hybridizes immediately adjacent to said polymorphic site. Therefore, polymerase-mediated extension of the primer by adding a single base, i.e., deoxynucleotide (dNTP) or dideoxynucleotide (ddNTP) (ddNTPs only allow extension by a single nucleotide), may provide information about which allele of the polymorphism is present in the PCR product, provided the identity of the added base can be determined. For example, in one method each ddNTP may contain a different fluorescent label, such that the identity of the incorporated ddNTP can be determined. In another exemplary method, the identity of the incorporated base is determined using fluorescence resonance energy transfer between a fluorescent label on the incorporated base and on the primer, such as described, e.g., by Chen et al. (PNAS 94: 10756-10761, 1997). In another method, identity of the incorporated base is determined by detecting the pyrophosphate by-product which is released during extension of the primer, such as in the method of pyrosequencing described by Ahmadian et al. (Anal Biochem 280: 103-110, 2000) and commercialized by Biotage AB (http://www.biotage.com/). Extended primers containing a polymorphism may be detected by mass spectrometry as described in U.S. Pat. No. 5,605,798.

Further, a polymorphism may also be tested in a segment of the ATF6-alpha gene or locus, either directly in the genetic material of a subject or after amplification from the genetic material (DNA and/or mRNA) of a subject by PCR, by oligonucleotide ligation assay (OLA) described by Landegren et al. (Science 241: 1077-1080, 1988) and further developed by, e.g., Eggerding et al. (Hum Mutat 5: 153-165, 1995) and Nickerson et al. (Proc Natl Acad Sci USA 87: 8923-8927, 1990). OLA is particularly suited for analyzing SNP polymorphisms. Here, a first primer is designed which hybridizes to the nucleic acid segment at and adjacent to a particular polymorphic site of interest in the ATF6-alpha gene or locus, such that the terminal 3′ nucleotide of the first primer is aligned with said polymorphic site. Therefore, the terminal 3′ nucleotide of the first primer may be chosen such that it will only base pair with a given allele of said polymorphism, but not with other alleles. A second primer common to all alleles is designed which hybridizes to the same strand of the nucleic acid segment as the first primer, but on the opposite side of the polymorphic site, such that the terminal 5′ nucleotide of the second primer is immediately adjacent to said polymorphic site. Therefore, if the terminal 3′ nucleotide of the first primer can base pair with the respective nucleotide of the nucleic acid segment, a ligase enzyme will catalyze a ligation reaction between the first and second primers, thereby producing a ligation product. The occurrence of such ligation product therefore indicates the presence in the subject of at least one allele of said polymorphism to which the terminal 3′ nucleotide of the first primer is complementary. Typically, OLA can be performed using repeated cycles of primer annealing and ligation, which will amplify the ligation product, thereby allowing the reaction to be performed directly on the genetic material of a subject (e.g., genomic DNA, cDNA). Exponential amplification of the ligation product is possible using ligase chain reaction (LCRB) described in Barany et al. (Proc Natl Acad Sci USA 88: 189-193, 1991). Usually, the first and second primer each carry a different detectable label and the presence of the ligation product is detected by co-occurrence of said labels in a single product. For example, such labels may be fluorescent, enzymatic, or radioactive.

Accordingly, the present invention relates to a method as described above, wherein said polymorphism is tested by a method chosen from the group comprising polymerase chain reaction-restriction fragment length polymorphism (PCR-RFLP), allele-specific amplification (ASA), hybridization with allele-specific oligonucleotides (ASO), single base extension (SBE) assay, oligonucleotide ligation assay (OLA), and sequencing.

As explained above, many methods for testing (i.e., genotyping) of polymorphisms require that a nucleic acid segment comprising a given polymorphism of interest is first amplified from the genetic material of subjects, e.g., from genomic DNA, mRNA or cDNA. Such segments can be most commonly amplified using polymerase chain reaction (PCR) disclosed in U.S. Pat. No. 4,965,188. To amplify a given segment of target nucleic acid, PCR employs a pair of oligonucleotide primers which are able to anneal to the borders of said segment, each of the primers being complementary to another of the two DNA strands of the segment, and oriented such that extension of each of the primers at its 3′-OH terminus by a DNA polymerase will synthesize the respective strand of the segment including the site complementary to the other primer (i.e., the binding site for that primer). Therefore, repeated cycles of template (i.e., target nucleic acid and PCR product) denaturation, primer annealing and primer extension by a DNA polymerase, typically a heat-resistant polymerase, such as Taq polymerase, will result in exponential amplification of the target nucleic acid segment.

Accordingly, the present invention relates to a method as described above, wherein said amplification is by PCR.

Other techniques for amplification of target nucleic acid sequences have also been developed any may be used in the present method of genotyping. For example, Walker et al. (U.S. Pat. No. 5,455,166; EP 0684315) described a method called strand displacement amplification (SDA), which differs from PCR in that it operates at a single temperature and uses a polymerase/endonuclease combination of enzymes to generate single-stranded fragments of the target DNA sequence, which then serve as templates for the production of complementary DNA strands. Another alternative amplification procedure, termed nucleic acid sequence-based amplification (NASBA) was disclosed by Davey et al. (U.S. Pat. No. 5,409,818; EP 0329822). Similar to SDA, NASBA employs an isothermal reaction, but is based on the use of RNA primers for amplification rather than DNA primers as in PCR or SDA.

Accordingly, genotyping of polymorphisms in the ATF6-alpha gene or locus of subjects in the present method of genetic diagnosis of insulin resistance phenotypes may require that at least part of the ATF6-alpha gene or locus or particular segments of the ATF6-alpha gene or locus, which comprise the polymorphisms of interest, are amplified from the genetic material (genomic DNA, mRNA, cDNA) of said subjects.

Accordingly, in an aspect, the present invention provides oligonucleotide primers, which are suitable for amplification of segments of the ATF6-alpha gene (genomic form, mRNA or cDNA) or locus by PCR, wherein said segments or parts comprise at least one polymorphism, the genotyping of which is useful in the present method of genetic diagnosis of insulin resistance phenotypes. For example, said polymorphism may preferably show genetic association with one or more insulin resistance phenotypes in at least one population or may belong to a group of polymorphisms which shows genetic association with one or more insulin resistance phenotypes in at least one population. The present invention further provides pairs of oligonucleotide primers which are suitable for amplification of said segments or parts by PCR. Preferably, at least one oligonucleotide is chosen from the group consisting of SEQ ID NO: 2 to 7.

Accordingly, in an embodiment the invention provides a set of two oligonucleotide primers suitable for amplification using polymerase chain reaction of a nucleic acid sequence from the ATF6-alpha gene or locus or ATF6-alpha cDNA comprising at least one polymorphism according to the invention, e.g., wherein said polymorphism can be tested in the present method of genetic diagnosis of insulin resistance phenotypes.

In a further embodiment, the present invention relates to a set of two oligonucleotide primers for amplification using polymerase chain reaction of a nucleic acid sequence from the ATF6-alpha gene or locus or ATF6-alpha cDNA comprising a particular allele of said at least one polymorphism according to the invention.

The term “oligonucleotide” as used herein refers to a molecule comprising two or more deoxyribonucleotides or ribonucleotides. These oligonucleotides may function as primers and probes.

The term “primer” as used herein refers to an oligonucleotide either naturally occurring (e.g. as a restriction fragment) or produced synthetically, which may act as a point of initiation of synthesis of a primer extension product and which is able to hybridize to a nucleic acid strand (template or target sequence) when placed under suitable conditions (e.g. buffer, salt temperature and pH) in the presence of nucleotides and an agent for nucleic acid polymerization, such as DNA dependent or RNA dependent polymerase. A primer must be sufficiently long to prime the synthesis of extension products in the presence of an agent for polymerization. A typical primer contains at least about 10 nucleotides in length of a sequence substantially complementary or homologous to the target sequence, but somewhat longer primers are preferred. The oligonucleotide primers may typically be between 10 and 35 nucleotides in length, preferably between 15 and 30 nucleotides in length, most preferably between 20 to 25 nucleotides long.

Normally a pair (or set) of oligonucleotide primers will consist of at least two primers, one “upstream” (or “forward”) and one “downstream” (or “reverse”) primer which together define the “amplificate” (the sequence that will be amplified using said primers). The pairs of oligonucleotide primers may be selected such that the resulting PCR amplification product may be between 50 bp and 2 kbp long, preferably between 100 bp to 1 kbp long, and more preferably between 150 bp and 500 bp long. The pairs of oligonucleotide primers may further be selected such that the two oligonucleotide primers of a given pair have similar melting temperatures (Tm), e.g., Tm of the primers differs by less than 10° C., preferably less than 5° C., more preferably less than 2° C. and most preferably be less than 1° C. Typically, the primers will be completely complementary to their binding sites in one or the other strand in the genetic material of subjects, but may comprise a certain number of mismatches (e.g., 1, 2, or 3 or more mismatches; e.g., primers may be at least 60%, preferably at least 70%, more preferably at least 80%, most preferably at least 90% or at least 95% identical to the complement of their binding sites; the percentage of identity may be determined using sequence alignment algorithm, e.g., BLASTN). Computerized algorithms for selection of suitable primers and primer pairs are available in the art. Primers may comprise additional sequences, e.g., on their 5′ sites, which are not complementary to the binding sites (e.g., 5′ overhangs with suitable restriction sites for cloning).

Further, the present invention also provides oligonucleotide primers suitable for genotyping of polymorphisms in the ATF6-alpha gene or locus using allele-specific amplification (ASA) as described elsewhere in this specification. Accordingly, in an embodiment the present invention provides a set of two oligonucleotide primers suitable for amplification using polymerase chain reaction specifically of a nucleic acid sequence from the ATF6-alpha gene (genomic form or cDNA) or locus comprising a particular allele of a polymorphism, wherein said polymorphism can be tested in the present method of genetic diagnosis of insulin resistance phenotypes. Preferably, the present invention relates to a method as described herein, wherein said PCR comprises the use of at least a set of two oligonucleotides, and said set is chosen from the group comprising: (i) SEQ ID NO: 2 and 3; (ii) SEQ ID NO: 4 and 5; and (iii) SEQ ID NO: 6 and 7.

As such it will be appreciated that the present invention relates also to the use of an oligonucleotide for the genetic diagnosis of an insulin resistance phenotype in a subject for at least one polymorphism in the gene or locus for activating transcription factor 6 alpha (ATF6-alpha), wherein said oligonucleotide hybridises specifically to a nucleic acid sequence comprising a particular allele of said at least one polymorphism as defined herein.

Further, the present invention also provides oligonucleotide primers suitable for genotyping of polymorphisms in the ATF6-alpha gene or locus using single-base extension (SBE) assay as described elsewhere in this specification, wherein said polymorphism can be tested in the present method of genetic diagnosis of insulin resistance phenotypes.

Further, the present invention also provides oligonucleotides primers suitable for genotyping of polymorphisms in the ATF6-alpha gene or locus using oligonucleotide ligation assay (OLA) as described elsewhere in this specification, wherein said polymorphism can be tested in the present method of genetic diagnosis of insulin resistance phenotypes.

In an embodiment, such primers will be suitable for genotyping the RS13401 SNP polymorphism, or the RS2070150 SNP polymorphism, or the RS1058405 SNP polymorphism in the ATF6-alpha gene or another polymorphism which is in LD with any of said polymorphisms.

In another aspect, the present invention provides oligonucleotide probes capable of specifically hybridizing to nucleic acid segments from the ATF6-alpha gene or locus. The oligonucleotide probes may typically be between 10 and 35 nucleotides in length, preferably between 15 and 30 nucleotides in length, most preferably between 15 and 25 nucleotides long. The oligonucleotide probes may be single-stranded and may comprise DNA, RNA, or PNA or combinations thereof. Typically, an oligonucleotide probe may be completely complementary to one or the other strand of a given sequence of the ATF6-alpha gene or locus in subjects, or may comprise a certain number of mismatches with said strand (e.g., 1, 2, or 3 or more mismatches; e.g., the probe may be at least 60%, preferably at least 70%, more preferably at least 80%, most preferably at least 90% or at least 95% identical to one or to the other strand of a given sequence of the ATF6-alpha gene or locus in subjects). It will be appreciated that oligonucleotide probes which should specifically hybridize to ATF6-alpha mRNA should be complementary to said mRNA. These oligonucleotide probes may be useful as allele-specific oligonucleotides (ASO) in genotyping of polymorphisms in the ATF6-alpha gene or locus, as described elsewhere in this specification. An ASO oligonucleotide probe will under stringent conditions hybridize specifically to a nucleic acid segment of the ATF6-alpha gene or locus which comprises a certain allele of a given polymorphism. Accordingly, in an embodiment, the present invention provides an oligonucleotide which hybridises specifically to a nucleic acid sequence comprising a particular allele of a polymorphism in the ATF6-alpha gene or locus, wherein said polymorphism can be tested in the present method of genetic diagnosis of insulin resistance phenotypes. Detection of such specific hybridization of an ASO probe to said segment therefore indicates the presence of said allele. A different (and differently labelled) ASO probe may be used to detect the other allele(s).

Hybridization of an allele-specific oligonucleotide (ASO) to a target polynucleotide may be performed with both entities in solution or such hybridization may be performed when either the oligonucleotide or the target polynucleotide is covalently or noncovalently affixed to a solid support. Attachment may be mediated, for example, by antibody-antigen interactions, poly-L-Lys, streptavidin or avidin-biotin, salt bridges, hydrophobic interactions, chemical linkages, UV cross-linking baking, etc. Allele-specific oligonucleotides may be synthesized directly on the solid support or attached to the solid support subsequent to synthesis. Solid-supports suitable for use in detection methods of the invention include substrates made of silicon, glass, plastic, paper and the like, which may be formed, for example, into wells (as in 96-well plates), slides, sheets, membranes, fibres, chips, dishes, and beads. The solid support may be treated, coated or derivatized to facilitate the immobilization of the allele-specific oligonucleotide or target nucleic acid.

Oligonucleotide primers (e.g., those for SBE) or probes (e.g., ASO probes) may be immobilized or synthesized on a solid support, such as on a microchip, beads, or a glass slide (see, e.g., WO 98/20020, WO 98/20019, WO 95/11995 and above). Different genotyping primers or probes may be ordered on a solid support in an array designed to rapidly screen a nucleic acid sample for one or more polymorphisms from the ATF6-alpha gene or locus and optionally for one or more polymorphisms from other genes, at the same time. Preferably, in the present invention such polymorphisms may be used in genetic diagnosis of insulin resistance diseases. For example, ASO probes may be used in sets wherein each ASO probe detects a different allele of a given polymorphism in the ATF6-alpha gene or locus. ASO probes of such set may be immobilized or synthesized at different locations or spots on the solid support. Moreover, ASO sets suitable for genotyping of different polymorphisms within the ATF6-alpha gene or locus may be immobilized or synthesized on the same solid support. Further, ASO sets for genotyping of polymorphisms in other genes or loci may also be included on other locations at the same solid support. For example, such polymorphisms in other genes may also be known to play a role in insulin resistance phenotypes. If a nucleic acid sample is hybridized with ASO probes immobilized or synthesized on a solid support, the nucleic acid sample will be suitably labelled in order to detect the hybridization. If SBE oligonucleotide primers are immobilized on a solid support and the SBE reaction is carried out, differently labelled ddNTP may preferably be used.

Accordingly, in an aspect, the present invention provides a solid support array comprising at least one oligonucleotide primer or probe capable of specifically hybridizing to a nucleic acid sequence of the ATF6-alpha gene or locus.

In an embodiment, said oligonucleotide probe may be an allele-specific probe (ASO) for a polymorphism in the ATF6-alpha gene or locus.

Accordingly, in an embodiment, the present invention provides an array, e.g., a solid support array, having immobilized thereon at least two oligonucleotides, wherein at least one of said oligonucleotides tests a polymorphism according to the invention, for instance, which hybridises specifically to a nucleic acid sequence comprising a particular allele of a polymorphism in the ATF6-alpha gene or locus.

In another embodiment, said array comprises at least two oligonucleotide probes, wherein each of said two oligonucleotides hybridizes specifically to a nucleic acid sequence of the ATF6-alpha gene comprising a different allele of a polymorphism.

Accordingly, in an embodiment, the present invention provides an array, e.g., a solid support array, having immobilized thereon at least two oligonucleotides, wherein said array comprises at least two oligonucleotides each of which hybridises specifically to a nucleic acid sequence comprising a different allele of a polymorphism in the ATF6-alpha gene or locus, wherein said polymorphism can be tested in the present method of genetic diagnosis of insulin resistance phenotypes.

It will be appreciated by the person skilled in the art that the arrays may comprise further oligonucleotides, such as control oligonucleotides, which may provide for positive and/or negative controls.

The oligonucleotide primers and/or probes of the present invention may comprise suitable labels to facilitate their detection. The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxygenin; luminogenic, phosphorescent or fluorogenic moieties; mass tags; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, characteristics of mass or behaviour affected by mass (e.g., MALDI time-of-flight mass spectrometry), and the like. A label may be a charged moiety (positive or negative charge) or alternatively, may be charge neutral. Labels can include or consist of nucleic acid or protein sequence, so long as the sequence comprising the label is detectable. Suitable fluorophore labels may include, for example, fluorescein, cascade blue, hexachloro-fluorescein, tetrachloro-fluorescein, TAMRA, ROX, Cy3, Cy3.5, Cy5, Cy5.5, 4,4-difluoro-5,7-diphenyl-4-bora-3a,4a-diaza-s-indacene-3-pro-pionic acid, 4,4-difluoro-5,p-methoxyphenyl-4-bora-3a,4a-diaza-s-indacene-3-propionic acid, 4,4-difluoro-5-styryl-4-bora-3a,4-adiaz-a-S-indacene-pro-pionic acid, 6-carboxy-X-rhodamine, N,N,N′,N′-tetramethyl-6-carboxyrhodamine, Texas Red, Eosin, fluorescein, 4,4-difluoro-5,7-diphenyl-4-bora-3a,4a-diaza-s-indacene-3-propionic acid, 4,4-difluoro-5,p-ethoxyphenyl-4-bora-3a-, 4a-diaza-s-indacene 3-propionic acid and 4,4-difluoro-5-styryl-4-bora-3a,-4-a-diaza-S-indacene-propionic acid.

Further, any of the above oligonucleotides, oligonucleotide primers, sets/pairs of oligonucleotide primers and/or oligonucleotide probes, which are suitable for (i) amplification of segments of ATF6-alpha gene or locus from genetic material (genomic DNA, mRNA, cDNA), wherein such segments comprise polymorphisms useful in the present method of genetic diagnosis of insulin resistance phenotypes; or for (ii) specific hybridization to nucleic acid sequences (e.g., in genetic material or amplified segments thereof) comprising said polymorphisms; or for (iii) specific hybridization to nucleic acid sequences (e.g., in genetic material or amplified segments thereof) comprising particular alleles of said polymorphism; may be provided as part of diagnostic kits for use in the method of genetic diagnosis of insulin resistance phenotypes. For example, such oligonucleotide primers or probes may be useful for testing, i.e., genotyping of said polymorphisms by PCR-RFLP, ASO hybridization, ASA, SBE, OLA, sequencing, etc. Kits may comprise one such oligonucleotide or more such oligonucleotides. Such oligonucleotides may be free (e.g., provided in solution or lyophilized) or may be immobilized on solid supports (e.g., microchips, glass slides, beads, etc.), e.g., in form of an array as described above. Such kits may provide suitable combinations of oligonucleotides, e.g., for detection of one or more polymorphisms in the ATF6-alpha gene or locus. As an illustrative example, a kit may comprise an oligonucleotide pair suitable for amplification of a given segment of the ATF6-alpha gene or locus, and another one or more oligonucleotides, e.g., ASO probes, suitable for detection of one or more alleles of a particular polymorphism in said segment. Alternatively, when oligonucleotides are immobilized on solid supports, such solid supports may form part of the kit and may comprise arrays of oligonucleotides suitable for detecting one or more alleles (or all alleles) of one or more polymorphism in the ATF6-alpha gene or locus and optionally in other genes or loci. It will be understood by a skilled person that oligonucleotides in such kits may be suitably labelled. Other components comprised in such kits may include, e.g., buffers, reagents, enzymes (e.g., polymerases, reverse transcriptases, ligases and the like), labelling reagents (e.g., to label a sample), instructions for use, etc. In particular, the present invention relates to a diagnostic kit for use in a method of genetic diagnosis of insulin resistance phenotypes, said kit comprising one or more oligonucleotide(s) and/or array(s) and/or set(s) of two oligonucleotides as defined herein.

As detailed in examples 3 and 4, it is another observation of the present invention that the amount of ATF6-alpha mRNA and ATF6-alpha protein in cultured pre-adipocytes from subjects having at least one G allele of the RS13401 polymorphism in the ATF6-alpha gene is higher than in subjects having two A alleles of said polymorphism. Because the presence of at least one G allele of the RS13401 polymorphism also correlates with increased susceptibility of subjects to develop insulin resistance and type 2 diabetes, it is a discovery of the present invention that subjects with increased levels of ATF6-alpha mRNA and/or protein may have increased susceptibility to develop insulin resistance phenotypes.

Accordingly, the present invention discloses that testing of subjects for the amount of ATF6-alpha mRNA and/or ATF6-alpha protein can be useful in diagnosis of insulin resistance phenotypes. In particular, increased levels of ATF6-alpha mRNA and/or ATF6-alpha protein may correlate with increased susceptibility of subjects to one or more insulin resistance phenotypes. By means of an example and not limitation, increased levels of ATF6-alpha mRNA and/or ATF6-alpha protein may correlate with increased susceptibility of subjects to develop insulin resistance; increased susceptibility of subjects to develop a disease of insulin resistance; increased likelihood for progression of subjects from insulin resistance to a disease of insulin resistance; increased rate of progression of subjects from insulin resistance to a disease of insulin resistance; faster progression of insulin resistance or of a disease of insulin resistance in subjects; increased severity of a disease of insulin resistance in subjects; increased response of subjects to a particular method of treatment (e.g., one directed at modulating the ATF6-alpha level or function or modulating cellular pathways involving ATF6-alpha).

Accordingly, in an aspect the present invention relates to a method of diagnosis of insulin resistance phenotypes, which comprises testing of a subject for the level of ATF6-alpha mRNA and/or ATF6-alpha protein. Preferably, said method for genetic diagnosis of an insulin resistance phenotype in a subject to be diagnosed comprises: (i) providing a sample of said subject to be diagnosed; and (ii) testing said sample for the level of ATF6-alpha mRNA and/or ATF6-alpha protein, wherein an increased level of ATF6-alpha mRNA and/or ATF6-alpha protein in said sample relative to the level of ATF6-alpha mRNA and/or ATF6-alpha protein of normal subjects diagnoses said subject to be diagnosed as having an insulin resistance phenotype, and wherein said insulin resistance phenotype is chosen from the group comprising insulin resistance syndrome (IRS), type 2 diabetes, metabolic syndrome, familial combined hyperlipidemia (FCHL), familial hyper-triglyceridemia, dyslipidemia, hepatic steatosis, obesity, polycystic ovary syndrome (PCOS), primary diabetes mellitus (DM), hypertension, lipodystrophy, fatty liver, hypercholesterolemia, inflammation and Cushing's syndrome.

The quantity of ATF6-alpha mRNA may be tested in a preparation comprising total RNA (tRNA) or mRNA of a subject isolated from a biological sample obtained from said subject. Total RNA may be isolated using standard methods, e.g., based on phenol-chloroform extraction or ion-exchange resins, and mRNA may be enriched, e.g., by selection of poly-A containing molecules using hybridization to poly-T oligonucleotides.

The quantity of ATF6-alpha mRNA in the preparation of tRNA or mRNA may be analyzed using methods known in the art, e.g., Northern blotting and detection with a labelled probe (e.g., oligonucleotide or polynucleotide) specifically hybridizing with ATF6-alpha mRNA; or quantitative RT-PCR or real-time quantitative RT-PCR, which will amplify a particular segment of ATF6-alpha mRNA. Real-time quantitative RT-PCR may be preferred due to its precision. To obtain an estimate of the amount of ATF6-alpha mRNA present in the sample, the signal obtained in the sample may be compared with a signal obtained in another sample comprising a known amount of ATF6-alpha mRNA or of another suitable DNA or RNA template when using RT-PCR. The signal may further be normalized with a constitutively expressed gene, e.g., beta-actin. These methods and considerations are known to the skilled artisan.

The quantity of ATF6-alpha protein may be tested in a preparation comprising proteins of a subject isolated from a biological sample obtained from said subject. Proteins may be isolated using standard methods, the choice of which may be dictated by the nature of the protein to be assayed and the assay method. Typically, the proteins will be isolated after lysis of the biological sample in a composition comprising appropriately selected ionic and/or non-ionic detergents.

The quantity of ATF6-alpha protein in the protein preparation may be analyzed using methods known in the art, e.g., Western blotting, immunoassay methods, e.g., radio immunoassay (RIA), enzyme linked immunosorbent assay (ELISA), immunoprecipitation, etc. Essentially all such methods involve the use of antibodies (polyclonal or monoclonal) specific for ATF6-alpha protein, which are available, such as, the polyclonal antibody “ATF-6α (H280)” available from Santa Cruz Biotechnology, Inc. (Santa Cruz, Calif., US) under catalogue number sc-22799; or the monoclonal anti-ATF6 antibody available under catalogue number IMG-273 from Imgenex Corp. (San Diego, Calif., US).

To obtain an estimate of the amount of ATF6-alpha protein present in the sample, the signal obtained in the sample may be compared with a signal obtained in another sample comprising a known amount of ATF6-alpha protein or of a suitable fragment thereof which is detected using the same antibody. The signal may further be normalized with a constitutively expressed gene, e.g., beta-actin. These methods and considerations are known to the skilled artisan.

It will be understood by a skilled person that any biological sample comprising cells which express the ATF6-alpha gene may be suitable to determine the quantity of ATF6-alpha mRNA and/or ATF6-alpha protein in a subject. Such biological sample may be, e.g., blood (containing lymphocytes) or a biopsy of skin tissue, muscle tissue, adipose tissue, liver tissue, etc. RNA or protein may be isolated directly from the sample or alternatively, cells from the sample may be first cultured and only afterwards used to isolate RNA or protein.

An aim of genetic association studies is to identify genes in which polymorphisms show genetic association with particular phenotypes, i.e., “susceptibility” genes for said phenotypes (in general, the term “susceptibility gene” is used in the art for a gene that is neither necessary nor sufficient to cause a particular phenotype but may, e.g., when comprising a particular sequence variation, increase or decrease the risk of subjects to develop such phenotype; herein, the term is not meant to exclude the possibility that major mutations in the ATF6-alpha gene or locus may exist which would almost invariably cause an insulin resistance phenotype). It is believed that proteins encoded by susceptibility genes participate in cellular pathways that, when not functioning optimally, lead to development of such phenotypes, e.g., diseases. Therefore, genetic association studies may discover novel cellular pathways involved in diseases and thereby provide one or more potential targets for therapeutic intervention. For example, such targets may encompass genes and gene products (e.g., mRNA and/or proteins) involved in said cellular pathways. In particular, such targets may include the susceptibility genes identified by genetic association studies and gene products (mRNA, proteins) thereof.

The present invention discloses that ATF6-alpha gene is a susceptibility gene for insulin resistance phenotypes and that polymorphisms in the ATF6-alpha gene or locus may show genetic association with one or more insulin resistance phenotypes. Accordingly, the present invention discloses that ATF6-alpha protein plays a crucial role in cellular events leading to development of insulin resistance and associated disease phenotypes, such as type 2 diabetes and metabolic syndrome.

Therefore, the present invention discloses that the ATF6-alpha gene or locus, ATF6-alpha mRNA and/or ATF6-alpha protein may represent potential therapeutic targets in treatment and/or prevention of insulin resistance phenotypes. Accordingly, in an aspect the present invention provides screening methods to identify agents, compounds or lead compounds for agents active at the level of a cellular function which can be modulated by ATF6-alpha.

In an embodiment, the screening methods involve assaying for compounds which modulate at least one aspect of the cellular function or metabolism of ATF6-alpha protein and/or which modulate cellular responses to ATF6-alpha protein. For example, under normal metabolic conditions in the cell, ATF6-alpha protein is located in the ER membrane as a type 11 transmembrane glycoprotein with apparent Mw of approximately 90 kD. However, upon endoplasmic reticulum (ER) stress, ATF6-alpha protein is cleaved and its N-terminal fragment of about 50 kD is soluble and localizes to the nucleus (Haze et al. Mol Biol Cell 10: 3787-3799, 1999). Accordingly, in an embodiment the screening methods may involve assaying for compounds which modulate, e.g., stimulate or inhibit, the cleavage of ATF6-alpha protein upon ER stress (ER stress can be induced experimentally in cells using a suitable agent, such as DTT, thapsigargin or tunicamycin).

Further, it is known that the cleavage of ATF6-alpha protein upon ER stress is catalyzed by site-1 and site-2 serine proteases (Ye et al. Mol Cell 6: 1355-1364, 2000), which also cleave sterol regulatory element-binding proteins in response to cholesterol deprivation. Accordingly, in another embodiment, the screening methods may involve assaying the effect of known inhibitors of site-1 and/or site-2 serine proteases on the ER-stress induced cleavage of ATF6-alpha protein.

Further, it is also known that after cleavage of ATF6-alpha protein upon ER stress, the N-terminal cleavage fragment of ATF6-alpha protein translocates to the nucleus (Haze et al. Mol Biol Cell 10: 3787-3799, 1999). Accordingly, in another embodiment, the screening methods may involve assaying for compounds which modulate, e.g., stimulate or inhibit, the nuclear translocation of the N-terminal ATF6-alpha fragment.

It is also known that in the nucleus the soluble N-terminal fragment of ATF6-alpha protein fragment binds to the ‘ER stress response element’ (ERSE) with consensus sequence CCAAT(N₉)CCACG or similar (see U.S. Pat. No. 6,635,751; Wang et al., J Biol Chem 275: 27013-27020, 2000) in the promoters of select genes, such as the genes encoding ER molecular chaperones GRP78 (HSPA5), GRP94 and calreticulin, and stimulates transcription of these genes. For example, overexpression of ATF6-alpha protein in human cells (e.g., HeLa) enhances transcription of these genes in an ERSE-dependent manner (Yoshida et al. J Biol Chem 273: 33741-33749, 1998). Accordingly, in another embodiment, the screening methods may involve assaying for compounds which modulate, e.g., stimulate or inhibit, (i) binding of ATF6-alpha protein or a biologically active fragment thereof (e.g., the soluble N-terminal fragment of about 50 kD) to nucleic acids (e.g. double-stranded oligonucleotides) comprising at least one (e.g., three or more) ERSE element and/or (ii) ATF6-alpha mediated transcription of ERSE-dependent genes, such as GRPs (e.g., HSPA5) or reporter genes controlled by promoters containing ERSE-elements.

Further, it is known that ATF6-alpha protein forms a heterodimer with ATF6-beta protein and further forms a heterotrimer with the nuclear transcription factor NF-Y. It is also known ATF6-alpha protein interacts with transcription factors GTF21, YY1 AND SRF. Accordingly, in another embodiment, the screening methods may involve assaying for compounds which modulate, e.g., stimulate or inhibit, these interactions.

Further, it is known that the N-terminal fragment of ATF6-alpha protein of about 50 kD (about 400 amino acids) which translocates to the nucleus upon ER stress, is rapidly degraded by the proteasome (Thuerauf et al. J Biol Chem 277: 20734-20739, 2002). Accordingly, in another embodiment, the screening methods may involve assaying for compounds which modulate, e.g., stimulate or inhibit, proteasomal degradation of this fragment.

In a general embodiment, the screening methods may involve assaying for compounds which bind to ATF6-alpha protein or to a biologically active fragment thereof.

In particular, the present invention relates to a method for identifying a molecule which binds to ATF6-alpha protein or a variant or biologically active fragment thereof, comprising the steps of: (a) incubating a mixture comprising ATF6-alpha protein or a variant or biologically active fragment thereof and at least one molecule; (b) allowing binding between said molecule and said ATF6-alpha protein or a variant or biologically active fragment thereof; and (c) determining binding between said molecule and said ATF6-alpha protein or a variant or biologically active fragment thereof. Preferably, said method further comprises identifying and/or isolating said molecule.

As used herein, the term “binding” refers to the physical association of a ligand, e.g. a molecule as defined herein, with a receptor, e.g. an ATF6-alpha protein or a variant or biologically active fragment thereof. As used herein the term is “specific” if it occurs with an EC₅₀ or a K_{d} of 100 nM or less, generally in the range of 100 nM to 10 pM. For example, binding is specific if the EC₅₀ or K_{d} is 100 nM, 50 nM, 10 nM, 1 nM, 950 pM, 900 pM, 850 pM, 800 pM, 750 pM, 700 pM, 650 pM, 600 pM, 600 pM, 550 pM, 500 pM, 450 pM, 400 pM, 350 pM, 300 pM, 250 pM, 200 pM, 150 pM, 100 pM, 75 pM, 50 pM, 25 pM, 10 pM or less. [30-32.] The present invention also relates to a method for identifying a molecule which modulates signal transduction mediated by ATF6-alpha protein, comprising the steps of: (a) incubating a mixture comprising ATF6-alpha protein or a variant or biologically active fragment thereof, a reporter construct comprising a reporter gene, wherein transcription of the reporter gene requires said ATF6-alpha protein or a variant or biologically active fragment thereof, and at least one molecule, under conditions allowing transcription of said reporter gene; and (b) determining if the latter incubation results in modulation of expression of said reporter gene compared to when said at least one molecule is absent. Preferably, said molecule increases or decreases expression of said reporter gene.

The tested compounds (i.e., candidate pharmacological agents) may encompass numerous chemical classes, though typically they are organic compounds or molecules; preferably small organic compounds or molecules and are obtained from a wide variety of sources including libraries of synthetic or natural compounds or molecules, or libraries of peptide molecules, or carbohydrates.

The above screening methods may employ ATF6-alpha protein or polypeptide. The term “ATF6-alpha protein” as used herein encompasses any polypeptide encoded by and/or expressed from ATF6-alpha gene in nature, i.e., in an organism. It will be appreciated that due to normal genetic variation, the amino acid sequences of ATF6-alpha proteins may differ, usually slightly, between individuals of a single species. The term “ATF6-alpha protein” further encompasses any polypeptide (e.g., polypeptide encoded by a recombinant nucleic acid and produced in transfected cells) having amino acid sequence identical to a polypeptide encoded by and/or expressed from ATF6-alpha gene in nature, i.e., in an organism. An exemplary human ATF6-alpha protein may have the amino acid sequence as encoded by the coding region (nucleotide 68 to 2080) comprised in the ATF6-alpha cDNA as shown in SEQ ID NO: 1. Other human ATF6-alpha proteins may differ from this sequence at e.g., 1 or more than one amino acid positions.

Such ATF6-alpha proteins or polypeptides may be provided, e.g., in an “isolated” or “substantially isolated” form, meaning that the protein or polypeptide has been separated from components which accompany it in its natural state. A monomeric protein is substantially pure when at least about 60 to 75% of a sample exhibits a single polypeptide sequence. A substantially pure protein will typically comprise about 60 to 90% w/w of a protein sample, more usually about 95%, and preferably will be over about 99% pure. Protein purity or homogeneity may be indicated by a number of means well known in the art, such as polyacrylamide gel electrophoresis of a protein sample, followed by visualizing a single polypeptide band upon staining the gel. For certain purposes, higher resolution may be provided by using HPLC or other means well known in the art which are utilized for purification.

The above screening methods may further employ functional variants of ATF6-alpha protein.

As used herein, the term “functional variants” of the ATF6-alpha protein encompass polypeptides or proteins which do not occur in nature and may differ from the from the ATF6-alpha protein as encoded by the cDNA sequence as shown in SEQ ID NO: 1 (FIG. 2) at one or more amino acid positions (e.g., at 1 to 10, or more than 10 amino acid positions), but which are functionally equivalent to ATF6-alpha protein in all aspects of its function, e.g., they will be properly localized to endoplasmic reticulum in unstressed cells; they will be cleaved and their N-terminal fragment will localize to nucleus in ER stressed cells, they will be able to stimulate expression of the same genes as ATF6-alpha, etc. For example, functional variants may contain amino acid substitutions, deletions or insertions at sites which are not crucial for ATF6-alpha protein function, or may comprise amino acid substitutions which replace an amino acid with a chemically similar amino acid (e.g., one having a hydrophobic side chain with another one having a hydrophobic side chain). Preferably, functional variants may show 80% or more, 90% or more, or 95% or more sequence identity with ATF6-alpha protein as encoded by the cDNA sequence as shown in SEQ ID NO: 1 (FIG. 2) (sequence identity may be determined, e.g., by aligning and comparison of two polypeptide sequences using the BLASTP algorithm, http://www.ncbi.nlm.nih.ciov/blast/).

Further, the term “functional variants” also encompasses polypeptides or proteins that are substantially homologous to primary structural sequence of ATF6-alpha protein but which include, e.g., in vivo or in vitro chemical and biochemical modifications or which incorporate unusual amino acids. Such modifications include, for example, acetylation, carboxylation, phosphorylation, glycosylation, ubiquitination, labeling, e.g., with radionuclides, and various enzymatic modifications, as will be readily appreciated by those well skilled in the art. A variety of methods for labelling polypeptides and of substituents or labels useful for such purposes are well known in the art, and include radioactive isotopes such as ¹²⁵I, ³²P, ligands which bind to labelled antiligands (e.g., antibodies), fluorophores, chemiluminescent agents, enzymes, and antiligands which can serve as specific binding pair members for a labelled ligand. The choice of label depends on the sensitivity required, ease of conjugation with the primer, stability requirements, and available instrumentation. Methods of labelling polypeptides are well known in the art. See, e.g., Sambrook et al., 1989 or Ausubel et al., 1992.

Further, the above screening methods may also employ biologically active fragments of ATF6-alpha or of functional variants thereof. A “fragment”, “portion” or “segment” of a polypeptide (e.g., ATF6-alpha protein) is a stretch of amino acid residues of at least about five to seven contiguous amino acids, often at least about seven to nine contiguous amino acids, typically at least about nine to 13 contiguous amino acids and, most preferably, at least about 20 to 30 or more contiguous amino acids.

A “biologically active” fragment is functionally equivalent to ATF6-alpha protein in at least one functional aspect. In particular, this may be an aspect which is being studied in a particular screening method. For example, an N-terminal fragment of ATF6-alpha which can localize to the nucleus and stimulate expression of target genes equivalent to the endogenous ATF6-alpha protein is considered herein biologically active fragment, even while it does not comprise the transmembrane portion for localization to EP. For example, such fragment may have about 373 N-terminal amino acids of ATF6-alpha. Other biological activities include ligand-binding or immunological activity. Immunological activities include both immunogenic functions in a target immune system, as well as sharing of immunological epitopes for binding, serving as either a competitor or substitute antigen for an epitope of the ATF6-alpha protein. As used herein, “epitope” refers to an antigenic determinant of a polypeptide. An epitope could comprise three amino acids in a spatial conformation which is unique to the epitope. Generally, an epitope consists of at least five such amino acids, and more usually consists of at least 8-10 such amino acids.

Further, ATF6-alpha protein or a variant or biologically fragment thereof may be provided as a fusion polypeptides with, e.g., a tag detectable by an antibody (e.g., His tag, HA tag, FLAG tag) or detectable using or allowing binding of the protein to specific molecules (e.g., GST tag, His tag), or detectable by other methods, e.g., fluorescence (e.g., GFP, RFP tag), or with a functional domain from another protein, e.g., a transactivation domain (e.g., Gal 4).

In an exemplary embodiment, the screening method may assay for compounds or molecules which bind to ATF6-alpha protein. Accordingly, the present invention provides a method for identifying a molecule which binds to ATF6-alpha protein or a variant or biologically fragment thereof comprising the steps of: (a) incubating a mixture comprising ATF6-alpha protein or a variant or biologically fragment thereof and at least one molecule; (b) allowing binding between ATF6-alpha protein or a variant or biologically fragment thereof; and (c) identifying and/or isolating said molecule. For example, the method may use ATF6-alpha protein or a variant or biologically fragment thereof free in solution, affixed to a solid support, borne on a cell surface, or expressed intracellularly. The method may use eukaryotic or prokaryotic host cells which are transiently or stably transformed with recombinant polynucleotides expressing ATF6-alpha protein or a variant or biologically fragment thereof. Ways of expressing such polypeptides are standard and involve transforming appropriate host cells with recombinant polynucleotide constructs, which comprise nucleic acid sequence coding for ATF6-alpha protein or a variant or biologically fragment thereof operably linked (the term “operably linked” refers to linkage of a DNA segment to another DNA segment in such a way as to allow the segments to function in their intended manners) to regulatory elements (e.g., promoter and enhancer elements) which can direct transcription of said nucleic acid sequence and expression of said encoded ATF6-alpha protein or a variant or biologically fragment thereof in said appropriate host cells. Such cells, either in viable or fixed form, can be used for standard binding assays. One may measure, for example, the formation of complexes between ATF6-alpha protein or a variant or biologically fragment thereof and the compound or molecule being tested, or examine the degree to which the formation of a complex between ATF6-alpha protein or a variant or biologically fragment thereof and a known ligand (e.g., ATF6-beta or a fragment thereof) is interfered with by the molecule or compound being tested. Such complexes may be detected using methods known in the art.

In another technique for high throughput screening for compounds or molecules having suitable binding affinity to the ATF6-alpha protein is described in detail in WO 84/03564. Briefly stated, large numbers of different small peptide test compounds are synthesized on a solid substrate, such as plastic pins or some other surface. The peptide test compounds are reacted with ATF6-alpha protein or a variant or biologically fragment thereof and washed. Bound ATF6-alpha protein or a variant or biologically fragment thereof is then detected by methods well known in the art, e.g. using antibodies.

Further, purified (i.e., isolated) ATF6-alpha protein or a variant or biologically fragment thereof can be coated directly onto plates for use in the aforementioned drug screening method. Non-neutralizing antibodies to the polypeptide can be used to capture antibodies to immobilize the ATF6-alpha protein or a variant or biologically fragment thereof on the solid phase.

In another exemplary embodiment, the screening method may assay for compounds or molecules which modulate signal transduction mediated by ATF6-alpha protein (i.e., activation of target gene expression by the N-terminal portion of ATF6-alpha protein upon ER stress). Accordingly, the present invention provides a method for identifying a molecule which modulates, e.g., stimulates or inhibits, signal transduction mediated by ATF6-alpha protein, comprising the steps of: (a) incubating a mixture comprising ATF6-alpha protein or a variant or biologically fragment thereof, a reporter construct comprising a reporter gene, wherein the transcription of the reporter gene requires said ATF6-alpha protein or a variant or biologically fragment thereof, and at least one molecule, and (b) determining if the latter incubation results in modulation of expression of said reporter gene compared to when said at least one molecule is absent.

Exemplary useful molecules may decrease or reduce the expression of a reporter gene by at least 30%, 40%, 50%, 60%, 70%, 80%, 90% or 100%, compared to the expression when said molecule is absent. In this regard, the term “expression” encompasses transcription and/or translation of said reporter gene.

In general, useful molecules, such as inhibitors, reduce or decrease the transcription, translation, stability and/or activity of ATF6-alpha or a variant or a biologically active fragment by at least 30%, 40%, 50%, 60%, 70%, 80%, 90% or 100%, compared to the transcription, translation and/or activity of ATF6-alpha or a variant or a biologically active fragment when said molecule is absent.

As mentioned above, “molecules” may also stimulate or increase the expression, such as, for instance, the transcription, translation, stability and/or activity of ATF6-alpha or a variant or a biologically active fragment. In this regard, molecules may stimulate or increase the expression by at least 30%, 40%, 50%, 60%, 70%, 80%, 90% or 100%, or at least 1.5 fold, 2 fold, 5 fold or more compared to the expression of ATF6-alpha or a variant or a biologically active fragment when said molecule is absent.

Assays to determine the transcription, translation, stability and/or activity of ATF6-alpha or a variant or a biologically active fragment are commonly known to the person skilled in the art. Several exemplary assays are detailed herein.

A “reporter gene” is a DNA molecule that expresses a detectable gene product, which may be RNA or protein. The detection may be accomplished by any method known in the art. Preferred reporter genes are those that are readily detectable, e.g., genes encoding chloramphenicol acetyl transferase (CAT), luciferase, beta-galactosidase and alkaline phosphatase. Reporter genes may be operably linked in a DNA construct (“reporter construct”) with a regulatory DNA sequence such that detection of the reporter gene product provides a measure of the transcriptional activity of the regulatory sequence.

Transcription of the reporter gene in the reporter construct requires ATF6-alpha protein or a variant or biologically fragment thereof, i.e., the regulatory elements which direct said transcription (e.g., promoter and enhancer) are controlled by ATF6-alpha protein or a variant or biologically fragment thereof. For example, suitable promoters may comprise one or more ERSE elements or similar elements, e.g., having a sequence as described in U.S. Pat. No. 6,635,751; Wang et al., J Biol Chem 275: 27013-27020, 2000. Such promoters may be derived from genes the expression of which is normally controlled by ATF6-alpha protein, e.g., Grp78, Grp94, or calreticulin. An exemplary suitable promoter for use in the reporter construct is described in Thuerauf et al. (J Biol Chem 23: 20734-20739, 2002).

The present method may best be performed in host cells or cell lines transfected with a suitable reporter construct. Such cells or cell lines may be, e.g., mammalian, preferably human cells or cell lines and may be primary or transformed cell lines (e.g., HeLa, 3T3, HepG2, COS, etc.). The cells or cell lines may be stably transfected or transiently transfected, as known in the art. The activity of the reporter gene may be assayed, e.g., in live cells, or fixed cells or in cell lysates.

It may be possible to perform the method using ATF6-alpha expressed endogenously by the cells. In such case it will be needed to induce ER stress, e.g., by DTT or thapsigargin, such that endogenous ATF6-alpha would be cleaved and would stimulate expression of the reporter gene. Alternatively, exogenous ATF6-alpha protein or a variant or biologically fragment thereof may be stably or transiently expressed in the cells from an appropriate expression construct. If so expressed ATF6-alpha protein or a variant or biologically fragment thereof is localized to ER, it may be necessary to induce ER stress. Otherwise, the biologically active fragment of ATF6-alpha protein or a variant thereof may only contain the N-terminal portion of ATF6-alpha protein (e.g., about 400 N-terminal amino acids, e.g., 373 or more N-terminal amino acids as done by Haze et al. Mol Biol Cell 10: 3787-3799, 1999) but not the remaining sequences responsible for localization to ER. Such fragment may direct expression of the reporter gene in the absence of ER stress. Moreover, ATF6-alpha protein or a variant or biologically fragment thereof used in the method may be fused to transactivation domains (i.e., domains recruiting the transcription machinery) derived from other proteins, e.g. from Gal 4.

The tested compound or molecule may be added to the culture medium of the cells a given time period before the test is carried out.

The aim of all above mentioned screening methods is to identify compounds or molecules which, by modulating the expression and/or function and/or metabolism of the ATF6-alpha protein, counteract insulin resistance and may thus be useful for treatment and/or prevention of insulin resistance phenotypes, e.g., insulin resistance and diseases of insulin resistance, including type 2 diabetes and metabolic syndrome.

Accordingly, compounds and molecules identified by these screening methods may further be tested, e.g., for their effects on ER stress, UPR and insulin signalling. In an exemplary test, a compound or molecule may be added to suitable cells (e.g., mammalian or human primary or transformed cell lines, e.g., HepG2 or 3T3) and ER stress may subsequently be induced in so-treated cells using, e.g. DTT or thapsigargin. Next, markers of ER stress and UPR can be measured, e.g., by Western blotting or quantitative RT-PCR, or other suitable techniques. Such markers may include, e.g., expression levels of GRP 78, GRP 94, and phosphorylation status of PERK and of elF2-alpha. A difference in these markers between cells treated with said compound or molecule and untreated cells may indicate that such treatment is able to modulate the ER stress and UPR response in cells.

Alternatively, markers of insulin signalling may be measured, e.g., phosphorylation status of IRS-1 (Insulin receptor substrate-1), IRS-2 (Insulin receptor substrate-2) and/or Akt/PKB (a major serine/threonine kinase mediating various insulin-dependent biological processes). For example, a compound or molecule which reduces phosphorylation of IRS-1 on Ser307 or of Akt on serine 473, or alternatively increases IRS (1 and/or 2) tyrosine phosphorylation, Insulin receptor tyrosine phosphorylation or total protein levels of the insulin receptor compared to untreated cells may be a useful candidate for treatment and/or prevention of insulin resistance phenotypes. As an alternative for phosphorylation, changes in total protein levels of, e.g., the insulin receptor, IRS-1, IRS-2, and/or Akt can be measured. A compound or molecule increasing total protein levels of, e.g., the insulin receptor, IRS-1, IRS-2, and/or Akt may be a useful candidate for treatment and or prevention of insulin resistance phenotypes

Alternatively to markers of insulin signalling, enzymes involved in gluconeogenesis may be measured. A compound or molecule decreasing levels of, e.g., the gluconeogenic enzymes phosphoenolpyruvate carboxykinase (PEPCK) or glucose-6-phosphatase (G6 Pase) may be a useful candidate for treatment and or prevention of insulin resistant phenotypes.

Further, the compounds and molecules identified by the above screening methods or derivatives thereof may also be tested in animal models of insulin resistance or diseases of insulin resistance, for their ability to ameliorate, treat or prevent such phenotypes, and may further be tested for efficacy and safety in animal and human trials. Further, such compounds and molecules or derivatives thereof may be formulated into pharmaceutical compositions as detailed elsewhere in this specification.

Accordingly, in an aspect the present invention relates to a method of treatment and/or prevention of one or more insulin resistance phenotypes, in particular insulin resistance and/or a disease of insulin resistance (e.g., metabolic syndrome, type 2 diabetes, obesity, heart failure, and familial combined hyperlipidemia) comprising administering to a subject in need of such treatment a therapeutically effective amount of molecule that binds to ATF6-alpha protein and/or modulates expression and/or function of ATF6-alpha gene or protein.

In another aspect, the present invention relates to a molecule that binds to ATF6-alpha protein and/or modulates expression and/or function of ATF6-alpha gene or protein for use as a medicament.

In a further aspect, the present invention relates to use of a molecule that binds to ATF6-alpha protein and/or modulates expression and/or function of ATF6-alpha gene or protein for the manufacture of a medicament to treat and/or prevent one or more insulin resistance phenotypes, in particular insulin resistance and/or a disease of insulin resistance (e.g., metabolic syndrome, type 2 diabetes, obesity, heart failure, or familial combined hyperlipidemia).

As detailed elsewhere in this specification, it is a discovery of the present invention that subjects with increased levels of ATF6-alpha mRNA and/or ATF6-alpha protein may have increased susceptibility to develop insulin resistance phenotypes, such as insulin resistance and diseases of insulin resistance. Accordingly, the present invention discloses that molecules which can antagonize expression and/or function of ATF6-alpha gene or protein may be useful for treatment and/or prevention of such insulin resistance phenotypes.

Accordingly, in an aspect the present invention relates to a method of treatment and/or prevention of one or more insulin resistance phenotypes, in particular insulin resistance and/or a disease of insulin resistance (e.g., metabolic syndrome, type 2 diabetes, obesity, heart failure, and familial combined hyperlipidemia) comprising administering to a subject in need of such treatment a therapeutically effective amount of molecule that antagonizes expression and/or function of ATF6-alpha gene or protein.

In another aspect, the present invention relates to a molecule that antagonizes expression and/or function of ATF6-alpha gene or protein for use as a medicament.

In a further aspect, the present invention relates to use of a molecule that antagonizes expression and/or function of ATF6-alpha gene or protein, such as for instance an inhibitor, for the manufacture of a medicament to treat and/or prevent one or more insulin resistance phenotypes, in particular insulin resistance syndrome and/or a disease of insulin resistance, in particular, wherein said diseases are chosen from the group consisting of insulin resistance syndrome (IRS), type 2 diabetes, metabolic syndrome, familial combined hyperlipidemia (FCHL), familial hyper-triglyceridemia, dyslipidemia, hepatic steatosis, obesity, polycystic ovary syndrome (PCOS), primary diabetes mellitus (DM), hypertension, lipodystrophy, Cushing's syndrome, fatty liver, inflammation, hypercholesterolemia, heart failure, atherosclerosis and cardiovascular diseases.

In a further preferred embodiment, said molecule, such as for instance an inhibitor is chosen from the group consisting of: anti-ATF6-alpha antibodies, functional fragments of anti-ATF6-alpha antibodies, antisense nucleic acids, siRNA molecules, ribozymes inhibiting translation of ATF6-alpha, dominant negative forms of ATF6-alpha protein, ATF6-beta protein or functional variants or fragments thereof.

In another preferred embodiment, said molecule, such as for instance an inhibitor decreases expression of ATF6-alpha, such as transcription, translation, and/or stability of the ATF6-alpha mRNA or protein, and/or inhibits the activity of ATF6-alpha, such as binding of ATF6-alpha to promoter sequences.

Such molecules may comprise, for example, anti-ATF6-alpha antibodies and functional fragments derived from such antibodies, antisense RNA and DNA molecules, small interfering RNA molecules, ribozymes that function to inhibit translation of ATF6-alpha, dominant negative forms of ATF6-alpha protein, ATF6-beta protein or functional variants or fragments thereof (e.g., Thuerauf et al., J Biol Chem 279: 21078-21084, 2004 reported that a soluble N-terminal fragment of ATF6-beta protein can in dominant negative manner inhibit the ATF6-alpha-mediated expression of target genes, e.g., Grp 78), and small molecules which bind to ATF6-alpha protein and/or interfere with signal transduction mediated by ATF6-alpha protein (i.e., activation of target gene expression by the N-terminal portion of ATF6-alpha protein upon ER stress). Further, small molecules can also interfere by binding on the promoter region of ATF6-alpha and inhibit binding of a transcription factor on said promoter region or said molecules can bind to ATF6-alpha protein itself and modulate its function as detailed above.

The term “antibody” or “antibodies” relates to an antibody characterized as being specifically directed against ATF6-alpha or any functional variant or biologically active fragment thereof, with said antibodies being preferably monoclonal antibodies; or an antigen-binding fragment thereof, of the F(ab′)₂, F(ab) or single chain Fv type, or any type of recombinant antibody derived thereof. These antibodies, including specific polyclonal antisera, have no cross-reactivity to other proteins. The monoclonal antibodies can for instance be produced by any hybridoma liable to be formed according to classical methods from splenic cells of an animal, particularly of a mouse or rat immunized against ATF6-alpha or any functional variant or biologically active fragment thereof, and of cells of a myeloma cell line, and to be selected by the ability of the hybridoma to produce the monoclonal antibodies specifically recognizing ATF6-alpha or any functional variant or biologically active fragment thereof which have been initially used for the immunization of the animals. The monoclonal antibodies according to this embodiment of the invention may be humanized versions of the mouse monoclonal antibodies made by means of recombinant DNA technology, departing from the mouse and/or human genomic DNA sequences coding for H and L chains or from cDNA clones coding for H and L chains. Alternatively the monoclonal antibodies may be human monoclonal antibodies. Such human monoclonal antibodies are prepared, for instance, by means of human peripheral blood lymphocytes (PBL) repopulation of severe combined immune deficiency (SCID) mice as described in PCT/EP 99/03605 or by using transgenic non-human animals capable of producing human antibodies as described in U.S. Pat. No. 5,545,806. Also fragments derived from these monoclonal antibodies such as Fab, F(ab)′2 and scFv (“single chain variable fragment”), providing they have retained the original binding properties, can be used. Such fragments are commonly generated by, for instance, enzymatic digestion of the antibodies with papain, pepsin or other proteases. In another embodiment of the invention the inhibitor of ATF6-alpha can be a camel antibody or a functional fragment thereof, e.g., as described in WO94/25591.

Oligonucleotide sequences, that include anti-sense RNA and DNA molecules and ribozymes may function to inhibit the translation of ATF6-alpha mRNA. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions of the ATF6-alpha mRNA (+1 corresponding to the A nucleotide of the ATG translation initiation codon and −1 corresponding to the 5′ nucleotide immediately adjacent to this A) are preferred. Ribozymes are enzymatic RNA molecules capable of catalyzing the specific cleavage of RNA. The mechanism of ribozyme action involves sequence specific hybridization of the ribozyme molecule to complementary target RNA, followed by a endonucleolytic cleavage. Within the scope of the invention are engineered hammerhead motif ribozyme molecules that specifically and efficiently catalyze endonucleolytic cleavage of ATF6-alpha RNA and mRNA sequences. Specific ribozyme cleavage sites within any potential RNA target are initially identified by scanning the target molecule for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays (RPA). Both anti-sense RNA and DNA molecules and ribozymes of the invention may be prepared by any method known in the art for the synthesis of RNA or DNA molecules. These include techniques for chemically synthesizing oligoribonucleotides or oligodeoxyribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize anti-sense RNA constitutively or inducible, depending on the promoter used, can be introduced transiently or stably into cell lines.

Small nucleic acid molecules, such as short interfering nucleic acid (siNA), short interfering RNA (siRNA), double-stranded RNA (dsRNA), micro-RNA (miRNA), and short hairpin RNA (shRNA) molecules capable of mediating RNA interference (RNAi) against ATF6-alpha gene expression may also be used. RNAi refers to the process of sequence-specific post-transcriptional gene silencing in animals mediated by short interfering RNAs (siRNAs).

The term “short interfering RNA” refers to an RNA (or RNA analog) comprising between about 10-50 nucleotides (or nucleotide analogs) which is capable of directing or mediating RNA interference. In preferred embodiments, an siRNA comprises about 15-30 nucleotides (or nucleotide analogs), 20-25 nucleotides (or nucleotide analogs), or 21-23 nucleotides (or nucleotide analogs). Unless otherwise indicated herein, the term siRNA refers to double stranded siRNA (as compared to single stranded or antisense RNA). The term “short hairpin RNA” (shRNA) refers to an siRNA (or siRNA analog) which is folded into a hairpin structure. shRNAs typically comprise about 45-60 nucleotides, including the approximately 21 nucleotide antisense and sense portions of the hairpin, optional overhangs on the non-loop side of about 2 to about 6 nucleotides long, and the loop portion that can be, e.g., about 3 to 10 nucleotides long. siRNAs can be unmodified or chemically-modified, and may be chemically synthesized, expressed from a vector in a cell, e.g., from a viral vector, or enzymatically synthesized, e.g., by in vitro transcription from a DNA template using a T7 or SP6 RNA polymerase. In one embodiment, the invention features a double-stranded short interfering nucleic acid (siNA), e.g., siRNA, molecule that down-regulates expression of ATF6-alpha gene, wherein said siNA molecule comprises about 15 to about 28 base pairs. In one embodiment, the invention features a double stranded short interfering nucleic acid (siNA) molecule, e.g., siRNA, that directs cleavage of ATF6-alpha RNA and/or mRNA via RNA interference (RNAi), wherein the double stranded siNA molecule comprises a first and a second strand, each strand of the siNA molecule is about 18 to about 28 nucleotides in length, the first strand of the siNA molecule comprises nucleotide sequence having sufficient complementarity to the ATF6-alpha RNA or mRNA for the siNA molecule to direct cleavage of the ATF6-alpha RNA or mRNA via RNA interference, and the second strand of said siNA molecule comprises nucleotide sequence that is complementary to the first strand. In embodiment, the siNA molecule, e.g., siRNA, may be used to specifically down regulate or inhibit the expression of ATF6-alpha protein encoded by mRNA comprising an allele of a polymorphism which is correlated with increased susceptibility of subjects to developing one or more insulin resistance phenotypes. For further details on design of siRNA agents, see Elbashir et al. (Nature 411: 494-501, 2001).

Molecules which bind to ATF6-alpha protein and/or modulate, e.g., antagonize, the expression and/or function of ATF6-alpha gene or protein, e.g., any molecule as described above or as identified by the screening methods described above or a derivative thereof, may be useful for the treatment and/or prevention of insulin resistance and/or diseases of insulin resistance. Efficacy and/or safety of such molecules may be further tested in human or animal trials, as known in the art.

ATF6-alpha, located on chromosome 1 is responsible for actual activation of the unfolded protein response, whereas ATF6-beta (CREBL1)—a different gene on chromosome 6— is opposing to ATF6-alpha and may serve as a transcriptional repressor, functioning in part to regulate the strength and duration of ATF6 alpha-mediated transcription in the unfolded protein response.

It will thus be appreciated by the person skilled in the art that the present invention relates to the use of an activator of ATF6-beta for the manufacture of a medicament to treat diseases related to insulin resistance. Preferably, said diseases are chosen from the group comprising insulin resistance syndrome (IRS), type 2 diabetes, metabolic syndrome, familial combined hyperlipidemia (FCHL), familial hyper-triglyceridemia, dyslipidemia, hepatic steatosis, obesity, polycystic ovary syndrome (PCOS), primary diabetes mellitus (DM), hypertension, lipodystrophy, Cushing's syndrome, fatty liver, inflammation, hypercholesterolemia, heart failure, atherosclerosis and cardiovascular diseases.

As used herein, “an activator of ATF6-beta” relates to a molecule that increases the transcription, translation, stability and/or activity of ATF6-beta by at least 30%, 40%, 50%, 60%, 70%, 80%, 90% or 100%, or at least 1.5 fold, 2 fold, 5 fold or more compared to the transcription, translation, stability and/or activity of ATF6-beta. Assays to determine the transcription, translation, stability and/or activity of ATF6-beta are commonly known to the person skilled in the art.

Such molecules may be administered as such or in form of pharmaceutically acceptable salts. Such molecules may be commonly administered together with pharmaceutically acceptable carrier or excipient (used interchangeably), i.e., in form of a pharmaceutical composition. Such compositions are usually referred to as “medicament” or “medicament to treat” and may be used to treat insulin resistance phenotypes as indicated above. The administration of a molecule or compound or a pharmaceutically acceptable salt thereof may be by way of oral, inhaled or parenteral administration. An amount effective to treat the phenotypes hereinbefore described depends on the usual factors such as the nature and severity of the disorders being treated and the weight of the mammal. However, a unit dose will normally contain 0.01 to 50 mg for example 0.01 to 10 mg, or 0.05 to 2 mg of the identified molecule or compound or a pharmaceutically acceptable salt thereof. Unit doses will normally be administered once or more than once a day, for example 2, 3, or 4 times a day, more usually 1 to 3 times a day, such that the total daily dose is normally in the range of 0.0001 to 1 mg/kg; thus a suitable total daily dose for a 70 kg adult is 0.01 to 50 mg, for example 0.01 to 10 mg or more usually 0.05 to 10 mg. Such compositions may be in the form of tablets, capsules, oral liquid preparations, powders, granules, lozenges, reconstitutable powders, injectable and infusable solutions or suspensions or suppositories or aerosols. Tablets and capsules for oral administration are usually presented in a unit dose, and contain conventional excipients such as binding agents, fillers, diluents, tabletting agents, lubricants, disintegrants, colourants, flavourings, and wetting agents. The tablets may be coated according to well-known methods in the art.

### EXAMPLES

### Example 1

### Genetic Association of RS13401 SNP with Insulin Resistance

The genotype of RS13401 SNP was investigated in 125 subjects representative for the general Dutch population (Caucasians) and thus not artificially enriched for IFG/diabetic subjects (i.e., the prevalence of IFG and diabetes in this sample is comparable with the prevalence in the general population, when selected from the same age-group). These subjects exhibited the following characteristics: age 51±11 years; body mass index (BMI) 25.4±3.9 kg/m²; fasting blood glucose 4.9±0.7 mmol/L; fasting plasma insulin 6.1±5.4 μU/mL; and HOMA IR 1.4±1.5. 87.5% of subjects had normal fasting glucose (NFG) levels and 11.5% had impaired fasting glucose (IFG) levels, i.e. >5.6 mmol/L in whole blood. Beyond the differences in fasting glucose and insulin levels between NFG and IFG subjects (4.7±0.45 mmol/L in NFG, 6.4±0.8 mmol/L in IFG; fasting insulin levels: 5.6±5 μU/mL in NFG, 10.5±7.7 μU/mL in IFG), IFG subjects were significantly older (50±11 years NFG; 57±8 years IFG), and had a higher BMI (25.1±3.9 kg/m² in NGF; 27.9±3.5 kg/m² in IGF). 105 subjects were classified as normoglycaemic (NFG), 9 subjects as having impaired fasting glucose (IFG) and 7 subjects as being diabetic (whole blood glucose>6.4 mmol/L). Glucose levels in 4 of the 125 subjects were unknown.

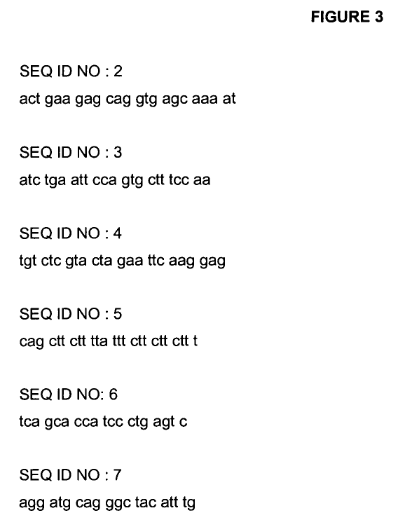

Genomic DNA was isolated from blood samples obtained from the subjects using routine methods. The segment of the ATF6-alpha gene comprising RS13401 SNP was amplified from the genomic DNA of the subjects by PCR using oligonucleotide primers having sequences as shown in SEQ ID NO: 2 (forward primer) and SEQ ID NO: 3 (reverse primer) (both in FIG. 3). The alleles of the RS 13401 SNP were determined in these PCR amplification products using single-base extension (SBE) assay with primers having sequences as shown in SEQ ID NO: 4 (sense orientation) and SEQ ID NO: 5 (antisense orientation) (both in FIG. 3) coupled to detection by fluorescence polarization.

Distribution of the alleles (“G” and “A” alleles) of the RS13401 SNP differed significantly between NFG and IFG/Diabetic subjects (see Table 1) and CHI square analysis produced a highly significant P-value (P=0.016). This result was confirmed in a logistic regression analysis, correcting for gender, age, and BMI (p=0.011). NFG subjects were in Hardy Weinberg Equilibrium (HWE) for RS13401 SNP, whereas IFG subjects were not.

TABLE 1
Distribution of RS13401 SNP genotypes between the IFG/diabetic and
normoglycaemic (NFG) subjects from a sample representative of the
general population. Please note that the total number of individuals in
this contingency table is 121, because glucose levels in 4 subjects
from the 125-subject sample were unknown.

| RS13401 genotype | normoglycaemic | IFG + diabetes |
|---|---|---|
| AA | 57.1% (N = 60) | 25% (N = 4) |
| AG + GG | 42.9% (N = 45) | 75% (N = 12) |

These results demonstrate that genetic variation in ATF6-gene regulates glucose metabolism in the non-diabetic range and that genetic association can exist between at least some polymorphisms in the ATF6-alpha gene or locus and insulin resistance, as evidenced by impaired glucose metabolism (IFG). A skilled geneticist will appreciate that it is unlikely that all polymorphisms in the ATF6-alpha gene or locus would show genetic association with insulin resistance phenotypes.

### Example 2

### Genetic Association of RS13401 SNP with Type 2 Diabetes

The genotype of RS13401 SNP was further investigated in a cohort of type 2 diabetes patients, IFG/IGT subjects and controls (N=797). For most of the type 2 diabetes patients, both fasting glucose concentration in plasma and glucose concentration in plasma 2 hours after consumption of the glucose solution in OGTT test were available. Subjects were classified according to the WHO guidelines for fasting glucose and glucose tolerance based on concentrations of glucose in plasma (i.e., not in whole blood). Subjects were diagnosed as type 2 diabetic (N=210) if their fasting plasma concentration of glucose was ≧7.0 mmol/L or their plasma glucose concentration 2 hours after consumption of the glucose solution in OGTT test was ≧11.1 mmol/L. To increase power, the type 2 diabetic subjects (N=210) were combined with a sample of subjects (N=208) showing impaired fasting glucose (IFG; herein fasting plasma glucose concentration ≧6.1 mmol/L and <7.0 mmol/L) or impaired glucose tolerance (IGT; herein fasting plasma glucose concentration <7.0 mmol/L and plasma glucose concentration 2 hours after consumption of the glucose solution in OGTT test ≧7.8 mmol/L). The control sample consisted of normoglycaemic subjects (N=379).

Genomic DNA was isolated from blood samples obtained from the subjects using routine methods. The segment of the ATF6-alpha gene comprising RS13401 SNP was amplified from the genomic DNA of the subjects by PCR using oligonucleotide primers having sequences as shown in SEQ ID NO: 6 (forward primer) and SEQ ID NO: 7 (reverse primer) (both in FIG. 3). The alleles of the RS 13401 SNP were determined in these PCR amplification products using restriction fragment length polymorphism (RFLP). Namely, the PCR amplification products of 299 bp were digested with the restriction enzyme Mnl I, and subsequently resolved on a 2% agarose gel. AA genotype is evidenced by fragments of 283 basepairs and 16 basepairs; AG genotype is evidenced by fragments of 283 base pairs, 143 basepairs and 16 basepairs; GG genotype is evidenced by fragments of 143 basepairs and 16 basepairs (typically, the 16 basepair fragment is too small to be visible on gel).

Distribution of RS13401 SNP genotypes between the (pre)diabetic samples (i.e., type 2 diabetes, IFG and IGT combined) and normoglycaemic samples are illustrated in Table 2. The distribution of RS13401 SNP genotypes differed significantly between the (pre)diabetic and normoglycaemic samples (CHI square analysis yielded a highly significant P-value, P=0.011). Odds ratio (OR) for the GG+GA group of genotypes (i.e., genotypes comprising at least one G allele) was OR=1.4 with a 95% CI 1.09-1.91.

TABLE 2
Distribution of RS13401 SNP genotypes between the (pre)diabetic
sample and normoglycaemic sample.

| RS13401 genotype | normoglycaemic | diabetes + IGT + IFG |
|---|---|---|
| AA | 60.2% (N = 228) | 51.2% (N = 214) |
| AG + GG | 39.8% (N = 151) | 48.8% (N = 204) |

These results demonstrate that subjects having at least one G allele of the RS13401 polymorphism have increased odds of developing insulin resistance (impaired glucose metabolism) and diseases of insulin resistance (e.g., type 2 diabetes) than subjects having the AA genotype. Hence, these results in general demonstrate that at least some polymorphisms in ATF6 gene or locus may show genetic association with insulin resistance and diseases of insulin resistance, e.g., type 2 diabetes, and that particular alleles of such polymorphisms may correlate with increased or decreased odds of subjects to display or develop such phenotypes. Testing of subjects for such polymorphisms may therefore be useful in genetic diagnosis of insulin resistance phenotypes. A skilled geneticist will appreciate that it is unlikely that all polymorphisms in the ATF6-alpha gene or locus would show genetic association with insulin resistance phenotypes.

### Example 3

### Increased ATF6-Alpha mRNA Levels in Subjects Having at Least One G Allele of the RS13401 SNP Polymorphism

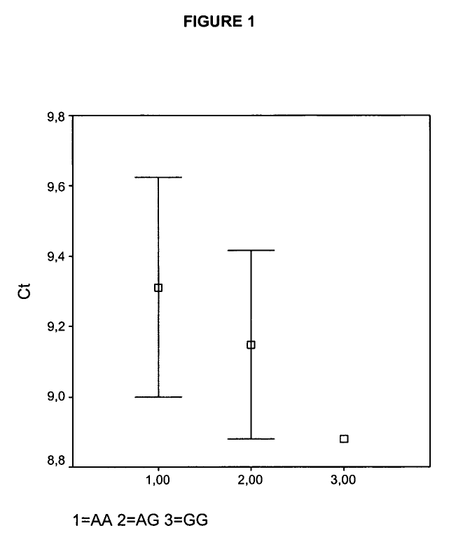

Pre-adipocytes obtained from subjects with the AA (N=20), AG (N=8), and GG (N=1) genotype of the RS13401 SNP polymorphism in the ATF6-alpha gene, were cultured under identical conditions and ATF6-alpha mRNA expression was measured using quantitative real time RT-PCR. The level of ATF6-alpha mRNA in subjects with M genotype was lower than in subjects with AG genotype, which in turn was lower than in the subject with GG genotype (FIG. 1). Please note that higher Ct-values in the graph of FIG. 1 reflect lower expression levels.

### Example 4

### Increased Levels of ATF6-Alpha Protein in Subjects Having Two G Alleles of the RS13401 SNP Polymorphism

Primary pre-adipocytes obtained from subjects with the AA (N=6) and GG (N=5) genotype of the RS13401 SNP polymorphism in the ATF6-alpha gene, are cultured under identical conditions, subsequently the proteins are isolated and the level of ATF6-alpha protein is measured by Western blotting using a specific anti-ATF6-alpha antibody. The levels are normalized to a constitutively expressed protein, e.g., beta-actin. The level of ATF6-alpha protein in cells from subjects having the GG genotype is on average higher than the level of ATF6-alpha protein in cells from subjects having the AA genotype.

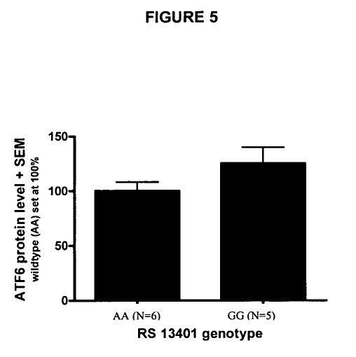

Specifically, a 25% increase was found in basal ATF6 protein expression in pre-adipocytes from the GG genotype compared to pre-adipocytes with the AA genotype (FIG. 5). A 25% increase or decrease in protein levels can have dramatic effects on the corresponding downstream pathway of that protein. This is especially true if it concerns a regulatory protein of a complex set of response genes that regulate a delicate homeostasis such as present in the endoplasmic reticulum. An excellent example to illustrate this concept comes from the diabetes field in a study by Mootha et al. (Nat Genet. 34:267-273, 2003), showing that a modest down-regulation (20%) of genes involved in oxidative phosphorylation in human diabetic muscle can actually explain a substantial part of the total-body-aerobic capacity (VO2max). Moreover they showed that a 20% decrease in one single transcription factor, i.e. PGC1, could be causal for these changes in the oxidative phosphorylation in diabetes.

### 4.1 Effect of Increased Levels of ATF6-Alpha Protein

The ATF6-alpha risk allele (RS13401, G-allele) is associated with increased ATF6alpha-RNA and ATF6-alpha protein levels. The effects of increased ATF6-alpha protein levels are assessed.

In order to study the effect of the levels of ATF6-alpha protein, ATF6-alpha protein levels are overexpressed according to the study by Kakiuchi et al. (Impaired feedback regulation of XBP1 as a genetic risk factor for bipolar disorder. Nat Genet. 35:171-175, 2003), which is specifically incorporated herein by reference. In short, lymphoblastoid cells are treated with low concentrations of the pharmaceutical agent valproic acid, thereby selectively increasing basal ATF6 levels, without affecting other members of the unfolded protein response pathway. When endoplasmic reticulum stress is subsequently induced in these cells, e.g. by tunicamycin, an enhanced activation of the unfolded protein response is observed.

In this regard it is noted that patients with epilepsy, bipolar disorder and schizophrenia are often treated with valproate. An interesting observation in this matter is the fact that major side effects of valproate treatment are weight gain, insulin resistance, fatty liver, dyslipidemia and type 2 diabetes (Pylvanen et al. Neurology. 2003 Feb. 25; 60(4):571-4; Luef et al. Ann Neurol. 2004 May; 55(5):729-32).

### Example 5

### Genetic Association of RS2070150 SNP with Familial Combined Hyperlipidemia (FCHL)

RS2070150 is a C/G substitution SNP polymorphism which results in a Pro/Ala change of amino acid 145 of the ATF6-alpha protein (RS2070150 corresponds to nucleotide position 500 of the ATF6-alpha cDNA sequence as shown in SEQ ID NO:1 in FIG. 2, where it is a G). The genotype of RS2070150 SNP was investigated in a case control study population which consisted of 77 genetically unrelated FCHL subjects (45 males, 32 females), each representing one FCHL family, and 125 unrelated spouses (50 males, 75 females). The spouses represent a common environment, nutrition and age matched control group for the FCHL subjects. The excess of spouse individuals is explained by (i) the inclusion of spouses whose partner had deceased due to complications of FCHL, and (ii) spouses of unaffected FCHL relatives, i.e., those with a fasting plasma cholesterol <6.5 mmol/l and a fasting plasma triglyceride level <2.3 mmol/l.

To establish the diagnosis of FCHL in a family, each of the following criteria were met: (i) a proband with a primary hyperlipidemia with varying phenotypic expression including fasting total plasma cholesterol >6.5 mmol/L (250 mg/dl) and/or a fasting plasma triglyceride concentration >2.3 mmol/l (200 mg/dl); (ii) at least one first degree relative with a different hyperlipidemic phenotype from the proband; (iii) a positive family history of premature cardiovascular disease. Premature cardiovascular disease was defined as the occurrence of a myocardial infarction or cerebrovascular incidence before the age of 60 years in at least one first degree relative of the proband, or the proband him/herself. Secondary causes of hyperlipidemia (hypothyroidism, renal or hepatic insufficiency), presence of the apo E2/E2 genotype (thus familial dysbetalipoproteinemia), and subjects with tendon xanthomas or a diagnosis matching familial hypercholesterolemia were excluded.

In a gender-stratified analysis (Table 3) there was a borderline significant association between RS2070150 SNP and FCHL affected status in females (P=0.06).

TABLE 3
Distribution of RS2070150 SNP genotypes between FCHL
affected females and control females.

| RS2070150 genotype | control sample female | FCHL affected female |
|---|---|---|
| GG | 58 (78.4%) | 29 (93.5%) |
| GC + CC | 16 (21.6%) | 2 (6.5%) |

These results demonstrate that female subjects having the GG genotype of RS2070150 SNP have increased odds of developing FCHL than female subjects having the GC or CC genotype.

### Example 6

### Genetic Association of RS1058405 SNP with Total Cholesterol and Apolipoprotein B Levels

RS1058405 is an A/G/T polymorphism which results in a Met/Val/Leu change of amino acid 67 of the ATF6-alpha protein (RS1058405 corresponds to nucleotide position 266 of the ATF6-alpha cDNA sequence as shown in SEQ ID NO: 1 in FIG. 2, where it is an A). This SNP is located in an 8-amino acid domain of ATF6-alpha protein (amino acid 61-68) that is 75% identical to the VN8 region of the virion protein 16 transcription factor (VP16) from herpes simplex virus type 1 (Thuerauf et al. J Biol Chem 277: 20734-20739, 2002). In VP16, these 8 amino acids, known as VN8 are required for transcriptional activation and rapid proteasomal degradation of VP16. Thuerauf et al. 2002 showed that the induction of point mutations in the VN8-like region of ATF6-alpha protein causes loss of transcription, increased expression levels, and an increase in half-life of the cleaved N-terminal part of ATF6-alpha protein. Therefore, the present inventors hypothesized (without being limited to any particular hypothesis) that the presence of a polymorphism in this region may affect stability of the cleaved (activated) p50 ATF6-alpha protein and therefore (given the effect of RS13401 SNP which seems to affect the stability of ATF6-alpha mRNA and is associated with insulin resistance phenotypes) that the RS1058405 polymorphism may also be associated with one or more insulin resistance phenotypes.

RS1058405 was significantly associated with total cholesterol and apolipoprotein B levels in a combined sample of FCHL patients (both males and females) and controls as detailed in example 6 (Table 4). Furthermore, the subjects having the GG-genotype showed on average higher BMI, waist, hip, waist hip ratio, systolic/diastolic blood pressure, triglycerides, insulin, and HOMA index (measure for insulin resistance).

TABLE 4
Association between total cholesterol or apolipoprotein
B levels and RS1058405 SNP polymorphism.

| genotype | N | Mean Chol +/− sd | Mean Apo B +/− sd |
|---|---|---|---|
| AA + GA | 175 | 6.0 +/− 1.3 | 1.13 +/− 0.34 |
| GG | 20 | 6.7 +/− 1.6 | 1.32 +/− 0.40 |
| | | P = 0.029 | P = 0.026 |

6.1 Further Assessment of the Genetic Association of RS1058405 SNP with Total Cholesterol and Apolipoprotein B Levels

The effect of the rs1058405 SNP on cholesterol levels was further investigated in part of the cohort of subjects described in Example 2. To exclude a possible interaction of this SNP with type 2 diabetes, subjects with type 2 diabetes were excluded. In 392 subjects without type 2 diabetes, the GG genotype was again associated with higher cholesterol levels (5.53±0.80 versus 5.21±0.90, p=0.04) and higher LDL cholesterol levels (3.72±0.80 versus 3.31±0.84, p=0.008) (see Table 5).

TABLE 5
Association between total cholesterol or apolipoprotein
B levels and the RS1058405 SNP polymorphism.

| Genotype | N | Mean chol ± sd | Mean LDL chol ± sd |
|---|---|---|---|
| AA + GA | 359 | 5.21 ± 0.90 | 3.31 ± 0.84 |
| GG | 33 | 5.53 ± 0.80 | 3.72 ± 0.80 |
| | | P = 0.04 | P = 0.008 |

6.2 Biological Support for the Genetic Association of ATF6-Alpha with Total Cholesterol and Apolipoprotein B Levels

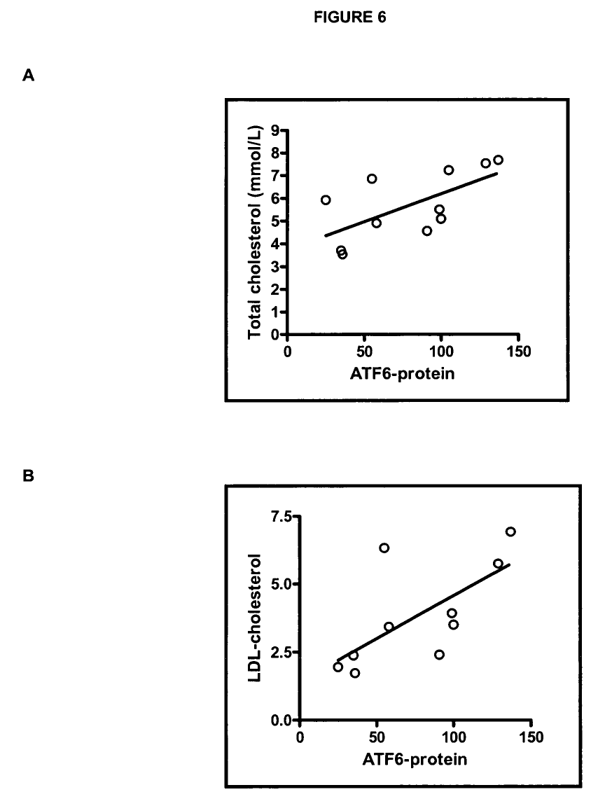

Primary human preadipocytes (n=11) were cultured under identical conditions. Proteins were isolated and the level of ATF6-alpha protein was measured by western blotting using a specific anti-ATF6-alpha antibody. ATF6-alpha levels were normalised to β-actin (x axis, arbitrary units) and plotted versus the plasma cholesterol and plasma LDL cholesterol levels (y-axis, mmol/L) that had been measured in the corresponding subjects, in vivo (FIG. 6).

A significant correlation was observed for ATF6-alpha protein levels with plasma cholesterol levels (FIG. 6A; r=0.65, p=0.03), and for ATF6-alpha protein levels with plasma LDL cholesterol levels (FIG. 6B; r=0.67, p=0.03).

6.3 Conclusion on the Genetic Association of ATF6-Alpha with Total Cholesterol and Apolipoprotein B Levels

These results demonstrate that RS1058405 is associated with insulin resistance phenotypes and/or with some aspects which play a role in insulin resistance phenotypes, e.g., total cholesterol and/or apolipoprotein B, which may play a role in, e.g., metabolic syndrome.

### Example 7

### Downregulation of ATF6-α by ATF6-β

It was hypothesized that ATF6-beta (CREBL1) opposes the activity of ATF6-alpha and may serve as a transcriptional repressor, functioning in part to regulate the strength and duration of ATF6 alpha-mediated transcription in the unfolded protein response.

In order to study the effects of ATF6-beta, N-terminal fragments of ATF6-alpha and ATF6-beta are overexpressed in HeLa cells and the effects on GRP78 induction are assessed according to the study by Thuerauf, et al. (J. Biol. Chem., Vol. 279:21078-21084, 2004: Opposing Roles for ATF6-alpha and ATF6-beta in ER stress response gene induction), which is incorporated herein by reference.

N-ATF6-beta inhibits N-ATF6-alpha-mediated GRP78 promoter activation in a dominant-negative manner. Thus, ATF6-beta may serve as a transcriptional repressor functioning in part to regulate the strength and duration of ATF6-alpha-mediated activation during the ER stress response. As such, increasing ATF6-beta (CREBL1) levels can be a method to prevent or downregulate activation of ATF6-alpha, and hence treat insulin resistance/diabetes.

### Example 8

### Downregulation of ATF6-Alpha by Antisense RNA

In order to downregulate the activity of ATF6-alpha, COS-1 cells (see below) are cotransfected with (i) an expression plasmid for ATF6-alpha; and (ii) antisense RNA complementary to various parts of the ATF6-alpha gene.

Culture methods and media for COS-1 monkey cells are as previously described by Kaufman (1997 Overview of vector design for mammalian gene expression. Methods Mol. Biol. 62: 287-300). Cells are transfected by FuGENE6 (Roche), according to the manufacturer's protocol.

The effect of antisense RNA is assessed by Western blots using anti-ATF6-alpha antibodies (e.g. purchased from Amersham Pharmacia; see also Example 6.2).

In a further experiment, the effect of antisense RNA is assessed by measuring luciferase, for which the cells are co-transfected with (i) an expression plasmid for ATF6-alpha; (ii) antisense RNA complementary to various parts of the ATF6-alpha gene; and (iii) a reporter plasmid containing the luciferase gene under control of five ATF6-binding sites.

The results demonstrate that antisense RNA downregulates the expression of ATF6-alpha, and can be used to treat insulin resistance phenotypes.

1. A method of genetic diagnosis of an insulin resistance phenotype in a subject, comprising testing of a sample from said subject for at least one polymorphism in the gene or locus for activating transcription factor 6 alpha (ATF6-alpha), wherein said insulin resistance phenotype is selected from the group consisting of insulin resistance syndrome (IRS), type 2 diabetes, metabolic syndrome, familial combined hyperlipidemia (FCHL), familial hyper-triglyceridemia, hypercholesterolemia, dyslipidemia, hepatic steatosis, obesity, polycystic ovary syndrome (PCOS), primary diabetes mellitus (DM), hypertension, inflammation, lipodystrophy, fatty liver and Cushing's syndrome.

2. The method according to claim 1, wherein said insulin resistance phenotype is insulin resistance syndrome (IRS) or type 2 diabetes.

3. The method according to claim 1, wherein said subject has a body mass index (BMI) of 25 kg/m² or more, or preferably 30 kg/m² or more.

4. The method according to claim 1, wherein said polymorphism is biallelic or multiallelic.

5. The method according to claim 1, wherein said polymorphism is a single nucleotide polymorphism (SNP).

6. The method according to claim 1, wherein said polymorphism in the ATF6-alpha gene or locus belongs to a group of two or more polymorphisms showing genetic association with one or more insulin resistance phenotypes in at least one population.

7. The method according to claim 6, wherein said group of two or more polymorphisms belongs to a single haplotype block.

8. The method according to claim 1, wherein said polymorphism is located in the exons, introns, coding region, 5′ untranslated region, 3′ untranslated region, enhancer region or promoter of the ATF6-alpha gene or locus.

9. The method according to claim 1, wherein said polymorphism is located in exon 3, exon 5 or the 3′ untranslated region of the ATF6-alpha gene or locus.

10. The method according to claim 1, wherein said polymorphism results in an amino acid substitution of the ATF6-alpha polypeptide as shown in SEQ ID NO: 8 illustrated in FIG. 4.

11. The method according to claim 1, wherein said polymorphism is selected from the group consisting of:
(i) an A/G substitution in the ATF6-alpha gene at a position corresponding to nucleotide 2204 in ATF6-alpha cDNA sequence as shown in SEQ ID NO: 1 illustrated in FIG. 2;
(ii) an A/G or an A/T substitution in the ATF6-alpha gene at a position corresponding to nucleotide 266 in ATF6-alpha cDNA sequence as shown in SEQ ID NO: 1 illustrated in FIG. 2;
(iii) a C/T substitution in the ATF6-alpha gene at a position corresponding to nucleotide 536 in ATF6-alpha cDNA sequence as shown in SEQ ID NO: 1 illustrated in FIG. 2; and
(iv) a C/G substitution in the ATF6-alpha gene at a position corresponding to nucleotide 500 in ATF6-alpha cDNA sequence as shown in SEQ ID NO: 1 illustrated in FIG. 2.

12. The method according to claim 1, wherein said polymorphism is tested in DNA or RNA selected from mRNA, or cDNA prepared from said RNA or mRNA.

13. The method according to claim 1, wherein said polymorphism is tested by a method selected from the group consisting of polymerase chain reaction-restriction fragment length polymorphism (PCR-RFLP), allele-specific amplification (ASA), hybridization with allele-specific oligonucleotides (ASO), single base extension (SBE) assay, oligonucleotide ligation assay (OLA), and sequencing.

14. A method for genetic diagnosis of an insulin resistance phenotype in a subject for at least one polymorphism in the gene or locus for activating transcription factor 6 alpha (ATF6-alpha), comprising:
(i) contacting an oligonucleotide with a sample from said subject under conditions allowing specific hybridization of said oligonucleotide to a nucleic acid in said sample, wherein said nucleic acid comprises at least part of the gene or locus for ATF6-alpha;
(ii) determining specific hybridization between said oligonucleotide and said nucleic acid, whereby an insulin resistance phenotype in a subject is diagnosed, wherein said insulin resistance phenotype is selected from the group consisting of insulin resistance syndrome (IRS), type 2 diabetes, metabolic syndrome, familial combined hyperlipidemia (FCHL), familial hyper-triglyceridemia, hypercholesterolemia, hepatic steatosis, dyslipidemia, obesity, polycystic ovary syndrome (PCOS), primary diabetes mellitus (DM), hypertension, inflammation, lipodystrophy, and Cushing's syndrome.

15. The method according to claim 14, wherein said nucleic acid is DNA or RNA selected from mRNA, or cDNA prepared from said RNA or mRNA.

16. The method according to claim 14, wherein said oligonucleotide tests polymorphisms selected from the group consisting of:
(i) an A/G substitution in the ATF6-alpha gene at a position corresponding to nucleotide 2204 in ATF6-alpha cDNA sequence as shown in SEQ ID NO: 1 illustrated in FIG. 2;
(ii) an A/G or an A/T substitution in the ATF6-alpha gene at a position corresponding to nucleotide 266 in ATF6-alpha cDNA sequence as shown in SEQ ID NO: 1 illustrated in FIG. 2;
(iii) a C/T substitution in the ATF6-alpha gene at a position corresponding to nucleotide 536 in ATF6-alpha cDNA sequence as shown in SEQ ID NO: 1 illustrated in FIG. 2; and
(iv) a C/G substitution in the ATF6-alpha gene at a position corresponding to nucleotide 500 in ATF6-alpha cDNA sequence as shown in SEQ ID NO: 1 illustrated in FIG. 2.

17. The method according to claim 1, wherein at least part of the gene or locus for ATF6-alpha comprising said polymorphism is amplified.

18. The method according to claim 17, wherein said amplification is by PCR.

19. The method according to claim 18, wherein said PCR comprises the use of at least one oligonucleotide as defined by any of SEQ ID NO:s 2 to 7.

20. The method according to claim 19, wherein said PCR comprises the use of at least a set of two oligonucleotides, and said set is selected from the group consisting of:
(i) SEQ ID NO: 2 and 3;
(ii) SEQ ID NO: 4 and 5; and
(iii) SEQ ID NO: 6 and 7.

21. The method according to claim 1, comprising an array having immobilized thereon at least two oligonucleotides, wherein at least one of said oligonucleotides tests said polymorphism.

22. The method according to claim 21, wherein said array comprises at least two oligonucleotides, wherein each of said two oligonucleotides hybridises specifically to a nucleic acid sequence comprising a different allele of said polymorphism.

23. A method for the genetic diagnosis of an insulin resistance phenotype in a subject for at least one polymorphism in the gene or locus for activating transcription factor 6 alpha (ATF6-alpha), which comprises hybridizing an oligonucleotide specifically to a nucleic acid sequence particular allele of said at least one polymorphism as defined in claim 1.

24. A set of two oligonucleotide primers for amplification using polymerase chain reaction of a nucleic acid sequence from the ATF6-alpha gene or locus or ATF6-alpha cDNA comprising at least one polymorphism as defined in claim 1.

25. A set of two oligonucleotide primers for amplification using polymerase chain reaction of a nucleic acid sequence from the ATF6-alpha gene or locus or ATF6-alpha cDNA comprising a particular allele of said at least one polymorphism as defined in claim 1.

26. A method for genetic diagnosis of an insulin resistance phenotype in a subject to be diagnosed comprising:
(i) providing a sample of said subject to be diagnosed; and
(ii) testing said sample for the level of ATF6-alpha mRNA and/or ATF6-alpha protein, wherein an increased level of ATF6-alpha mRNA and/or ATF6-alpha protein in said sample relative to the level of ATF6-alpha mRNA and/or ATF6-alpha protein of normal subjects diagnoses said subject to be diagnosed as having an insulin resistance phenotype, and wherein said insulin resistance phenotype is elected from the group consisting of insulin resistance syndrome (IRS), type 2 diabetes, metabolic syndrome, familial combined hyperlipidemia (FCHL), familial hyper-triglyceridemia, hypercholesterolemia, hepatic steatosis, dyslipidemia, obesity, polycystic ovary syndrome (PCOS), primary diabetes mellitus (DM), hypertension, inflammation, lipodystrophy, fatty liver and Cushing's syndrome.

27. A diagnostic kit for use in a method of genetic diagnosis of insulin resistance phenotypes, said kit comprising one or more oligonucleotide(s) and/or array(s) and/or set(s) of two oligonucleotides as defined in claim 21.

28. A method for identifying a molecule which binds to ATF6-alpha protein or a variant or biologically active fragment thereof, comprising the steps of:
(a) incubating a mixture comprising ATF6-alpha protein or a variant or biologically active fragment thereof and at least one molecule;
(b) allowing binding between said molecule and said ATF6-alpha protein or a variant or biologically active fragment thereof; and
(c) determining binding between said molecule and said ATF6-alpha protein or a variant or biologically active fragment thereof.

29. The method according to claim 28, further comprising identifying and/or isolating said molecule.

30. A method for identifying a molecule which modulates signal transduction mediated by ATF6-alpha protein, comprising the steps of:
(a) incubating a mixture comprising ATF6-alpha protein or a variant or biologically active fragment thereof, a reporter construct comprising a reporter gene, wherein transcription of the reporter gene requires said ATF6-alpha protein or a variant or biologically active fragment thereof, and at least one molecule, under conditions allowing transcription of said reporter gene; and
(b) determining if the latter incubation results in modulation of expression of said reporter gene compared to when said at least one molecule is absent.

31. The method according to claim 30, wherein said molecule increases expression of said reporter gene.

32. The method according to claim 30, wherein said molecule decreases expression of said reporter gene.

33-39. (canceled)


SEQUENCE LISTING (8 sequences)

SEQ ID NO: 1 - DNA, 2488 bp, Homo sapiens
ttttgtccgcctgccgccgccgtcccagatattaatcacggagttccagggagaaggaacttgtgaaatgggggagccggctggggttgccggcaccatggagtcaccttttagcccgggactctttcacaggctggatgaagattgggattctgctctctttgctgaactcggttatttcacagacactgatgagctgcaattggaagcagcaaatgagacgtatgaaaacaattttgataatcttgattttgatttggatttgatgccttgggagtcagacatttgggacatcaacaaccaaatctgtacagttaaagatattaaggcagaacctcagccactttctccagcctcctcaagttattcagtctcgtctcctcggtcagtggactcttattcttcaactcagcatgttcctgaggagttggatttgtcttctagttctcagatgtctcccctttccttatatggtgaaaactctaatagtctctcttcagcggagccactgaaggaagataagcctgtcactggtcctaggaacaagactgaaaatggactgactccaaagaaaaaaattcaggtgaattcaaaaccttcaattcagcccaagcctttattgcttccagcagcacccaagactcaaacaaactccagtgttccagcaaaaaccatcattattcagacagtaccaacgcttatgccattggcaaagcagcaaccaattatcagtttacaacctgcacccactaaaggccagacggttttgctgtctcagcctactgtggtacaacttcaagcacctggagttctgccctctgctcagccagtccttgctgttgctgggggagtcacacagctccctaatcacgtggtgaatgtggtaccagccccttcagcgaatagcccagtgaatggaaaactttccgtgactaaacctgtcctacaaagtaccatgagaaatgtcggttcagatattgctgtgctaaggagacagcaacgtatgataaaaaatcgagaatccgcttgtcagtctcgcaagaagaagaaagaatatatgctagggttagaggcgagattaaaggctgccctctcagaaaacgagcaactgaagaaagaaaatggaacactgaagcggcagctggatgaagttgtgtcagagaaccagaggcttaaagtccctagtccaaagcgaagagttgtctgtgtgatgatagtattggcatttataatactgaactatggacctatgagcatgttggaacaggattccaggagaatgaaccctagtgtgagccctgcaaatcaaaggaggcaccttctaggattttctgctaaagaggcacaggacacatcagatggtattatccagaaaaacagctacagatatgatcattctgtttcaaatgacaaagccctgatggtgctaactgaagaaccattgctttacattcctccacctccttgtcagcccctaattaacacaacagagtctctcaggttaaatcatgaacttcgaggatgggttcatagacatgaagtagaaaggaccaagtcaagaagaatgacaaataatcaacagaaaacccgtattcttcagggtgctctggaacagggctcaaattctcagctgatggctgttcaatacacagaaaccactagtagtatcagcaggaactcagggagtgagctacaagtgtattatgcttcacccagaagttatcaagacttttttgaagccatccgcagaaggggagacacattttatgttgtgtcatttcgaagggatcacctgctgttaccagctaccacccataacaagaccacaagaccaaaaatgtcaattgtgttaccagcaataaacataaatgagaatgtgatcaatgggcaggactacgaagtgatgatgcagattgactgtcaggtgatggacaccaggatcctccatatcaaaagttcgtcagttcctccttacctccgagatcagcagaggaatcaaaccaacaccttctttggctcccctcccgcagccacagaggcaacccacgttgtcagcaccatccctgagtcattacaatagcaccctgcagctatgctggaaaactgagcgtgggaccctgccagactgaagagcaggtgagcaaaatgctgctttctgccttggtggcaggcagagaactgtctcgtactagaattcaaggaggaaagaagaagaaataaaagaagctgctccatttttcatcatctacccatctatttggaaagcactggaattcagatgcaagagaacaatgtttcttcagtggcaaatgtagccctgcatcctccagtgttacctggtgtagatttttttttctgtacctttctaaacctctcttccctctgtgatggttttgtgtttaaacagtcatcttcttttaaataatatccacctctcctttttgccatttcacttattgattcataaagtgaattttatttaaagcta

SEQ ID NO: 2 - DNA, 23 bp, Artificial
  DNA primer
actgaagagcaggtgagcaaaat

SEQ ID NO: 3 - DNA, 23 bp, Artificial
  DNA primer
atctgaattccagtgctttccaa

SEQ ID NO: 4 - DNA, 24 bp, Artificial
  DNA primer
tgtctcgtactagaattcaaggag

SEQ ID NO: 5 - DNA, 25 bp, Artificial
  DNA primer
cagcttcttttatttcttcttcttt

SEQ ID NO: 6 - DNA, 19 bp, Artificial
  DNA primer
tcagcaccatccctgagtc

SEQ ID NO: 7 - DNA, 20 bp, Artificial
  DNA primer
aggatgcagggctacatttg

SEQ ID NO: 8 - protein, 670 aa, Homo sapiens
MGEPAGVAGTMESPFSPGLFHRLDEDWDSALFAELGYFTDTDELQLEAANETYENNFDNLDFDLDLMPWESDIWDINNQICTVKDIKAEPQPLSPASSSYSVSSPRSVDSYSSTQHVPEELDLSSSSQMSPLSLYGENSNSLSSAEPLKEDKPVTGPRNKTENGLTPKKKIQVNSKPSIQPKPLLLPAAPKTQTNSSVPAKTIIIQTVPTLMPLAKQQPIISLQPAPTKGQTVLLSQPTVVQLQAPGVLPSAQPVLAVAGGVTQLPNHVVNVVPAPSANSPVNGKLSVTKPVLQSTMRNVGSDIAVLRRQQRMIKNRESACQSRKKKKEYMLGLEARLKAALSENEQLKKENGTLKRQLDEVVSENQRLKVPSPKRRVVCVMIVLAFIILNYGPMSMLEQDSRRMNPSVSPANQRRHLLGFSAKEAQDTSDGIIQKNSYRYDHSVSNDKALMVLTEEPLLYIPPPPCQPLINTTESLRLNHELRGWVHRHEVERTKSRRMTNNQQKTRILQGALEQGSNSQLMAVQYTETTSSISRNSGSELQVYYASPRSYQDFFEAIRRRGDTFYVVSFRRDHLLLPATTHNKTTRPKMSIVLPAININENVINGQDYEVMMQIDCQVMDTRILHIKSSSVPPYLRDQQRNQTNTFFGSPPAATEATHVVSTIPESLQ